SEQUENCE LISTING (7 sequences)

SEQ ID NO: 1 - DNA, 480 bp, Homo sapiens
ctcaagatcgcattcatgcgtcttcacctggaaggggtccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcacgcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccatggggatatgtgtgtgcgtgcatgcagcacacacacattcctttattttggattcaatcaagttgatcttcttgtgcacaaatcagtgcctgtcccatctgca

SEQ ID NO: 2 - DNA, 420 bp, Homo sapiens
tttggatcagtagtcactaacgttcgccagccataagtcctcgacgtggagaggctcagagcctggcatgaacatgaccctgaattcggatgcagagcttcttcccatgatgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccagacattctgggt

SEQ ID NO: 3 - DNA, 360 bp, Homo sapiens
gcctggcatgaacatgaccctgaattcggatgcagagcttcttcccatgatgatctgtccctcacagcagggtcttctctgtttcagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtaaggaggtggctttaggtcagccagcattttcctgacaccagggaccaggctgccttcccactagctgtattgtttaacacatgcaggggaggatgctctccagacattctgggt

SEQ ID NO: 4 - DNA, 360 bp, Homo sapiens
attgactctaagaggaaagatgaagtactatgttttaaagaatattatattacagaattatagaaattagatctcttacctaaactcttcataatgcttgctctgataggaaaatgagatctactgttttcctttacttactacacctcagatatatttcttcatgaagacctcacagtaaaaataggtgattttggtctagctacagtgaaatctcgatggagtgggtcccatcagtttgaacagttgtctggatccattttgtggatggtaagaattgaggctatttttccactgattaaatttttggccctgagatgctgctgagttactagaaagtcattgaaggtctcaactata

SEQ ID NO: 5 - DNA, 360 bp, Homo sapiens
cagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgacc

SEQ ID NO: 6 - DNA, 420 bp, Homo sapiens
tgggcttgaatagttagatgcttatttaaccttggcaatagcattgcattccctgtggtttttaataaaaattgaacttccctccctccctgcccccttaccctccacacccccaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacaggtactaggagcattattttctctgaaaggatgatctttgtgttctgaatctttatggggaaatgaggttaccacactagggaagatagagctttttaattatgggaagagttggttttaggttgt

SEQ ID NO: 7 - DNA, 420 bp, Homo sapiens
gtgtttttgcgttctctagtcactttaagaaccaaatggaaggtcacactagggttttcatttccattgattatagaaagctttaaagtactgtagatgtggctcgccaattaaccctgattactggtttccaacaggttcttgctggtgtgaaatgactgagtacaaactggtggtggttggagcaggtggtgttgggaaaagcgcactgacaatccagctaatccagaaccactttgtagatgaatatgatcccaccatagaggtgaggcccagtggtagcccgctgacctgatcctgtctctcacttgtcggatcatctttacccatattctgtattaaaggaataagaggagagaaagtaaaaagttattttgggtatacattcagttatgcaataagcttaacgtgtttatagag

(19) United States
(12) Patent Application Publication
(10) Pub. No.: US 2021/0324461 A1
(43) Pub. Date: Oct. 21, 2021

(54) REAGENTS, MIXTURES, KITS AND METHODS FOR AMPLIFICATION OF NUCLEIC ACIDS

(71) Applicant: Life Technologies Corporation, Carlsbad, CA (US)

(72) Inventors: Joyce WILDE, Moss Beach, CA (US); Shoulian DONG, Mountain View, CA (US); Carentine LAU, Belmont, CA (US); Junko STEVENS, Menlo Park, CA (US); Carmen GJERSTAD, Millbrae, CA (US)

(21) Appl. No.: 17/250,610

(22) PCT Filed: Aug. 16, 2019
(86) PCT No.: PCT/US2019/046950

§ 371 Date: Feb. 10, 2021

Related U.S. Application Data
(60) Provisional application No. 62/719,074, filed on Aug. 16, 2018.

(51) Int. Cl. C12Q 1/6858; C12Q 1/6886

(52) U.S. Cl. CPC C12Q 1/6858; C12Q 2600/156; C12Q 1/6886

(57) ABSTRACT

This disclosure relates to reagents, mixtures, kits and methods for use in detecting low-frequency target polynucleotides, such as rare allelic variants.

### CROSS-REFERENCE TO RELATED APPLICATIONS

This application claims priority to U.S. Provisional Application No. 62/719,074, filed Aug. 16, 2018, the disclosure of which is incorporated herein by reference in its entirety.

### FIELD OF THE DISCLOSURE

This disclosure relates to reagents, mixtures, kits and methods for use in detecting target polynucleotides, particularly mutant or low abundance target polynucleotides.

### BACKGROUND OF THE DISCLOSURE

In recent years, progress has been made in detecting and treating cancer. This progress is due in large part, to our deepening understanding of the molecular basis of cancer and the detailed molecular mechanisms used by cancer cells to evade the immune system. Thus, biomarkers have been identified that help to identify patients who will respond to certain cancer treatments. For example, biomarkers found in tumor cells, such as Programmed death-ligand 1 (PD-L1), identify patients who will respond to immunotherapies, such as anti-PD-1 antibodies. Furthermore, biomarkers include specific mutations in certain genes. For example, detection of mutations in certain kinases can help identify patients who are much more likely to respond to kinase inhibitor cancer therapeutics. Then, as cancers further mutate to overcome the block of a first kinase, detection of new mutations that give the cancer the ability to overcome this first kinase, can now be used to identify a next line of therapy that can be effective for this type of cancer.

The ability to detect mutations in cancer requires improved methods for detecting low abundance alleles. Low abundance allele detection is important when identifying the genetic defects underlying a tumor because of heterogeneity in cancerous cells that make up the tumors. Furthermore, low abundance allele detection is important in analysis of circulating DNA of cancer patients due to the presence of a relatively low amount of circulating tumor DNA compared to circulating DNA from normal cells. In both contexts, the ability to better detect such low abundance alleles should lead to improved detection and improved targeted treatment of cancer patients. Currently available systems are either not capable of detecting target polynucleotides (e.g., comprising mutant nucleic acid sequences) in very low copy numbers (e.g., less than 10 copies and/or less than 0.1% in a 30 ng test nucleic acid sample) or are extremely costly and/or time consuming and complicated. Thus, there is a need in the art for simplified and affordable reagents, mixtures, kits and methods for use in detecting low abundance target polynucleotides.

### SUMMARY OF THE DISCLOSURE

Provided herein are reagents, mixtures, kits and methods for use in detecting low abundance target polynucleotides. In some embodiments, this disclosure relates to mixtures comprising: a) a first oligonucleotide configured to hybridize to a first sequence in a first target polynucleotide strand (such as a first strand in a double-stranded polynucleotide), wherein the first sequence in the first target polynucleotide strand has a target variant nucleotide, and wherein the first oligonucleotide further has a nucleotide residue at its 3′-end that is positioned to hybridize to the target variant nucleotide; b) a second oligonucleotide having a sequence configured to hybridize to a sequence complementary to a second sequence (e.g., to a portion of a second strand that is complementary to the first strand) of the first target polynucleotide strand, wherein the second sequence of the first target polynucleotide strand is located 5′ upstream from the first sequence of the first target polynucleotide strand; and, c) a third oligonucleotide having a sequence configured to hybridize to a sequence complementary to a third sequence of the first target polynucleotide strand, wherein the third sequence of the first target polynucleotide strand overlaps at least partially with the first sequence of the first target polynucleotide strand and the target variant nucleotide. In some embodiments, additional oligonucleotides and/or sets of oligonucleotides are also provided. In some embodiments, the third oligonucleotide is detectable. The reagents combined to provide such mixtures are also contemplated herein, as are kits and methods comprising and/or using the same. Further details regarding aspects and embodiments of the present disclosure are provided throughout this patent application. Sections and section headers are not intended to limit combinations of methods, compositions, and kits or functional elements therein.

### BRIEF DESCRIPTION OF THE DRAWINGS

FIG. 1. Exemplary first oligonucleotide (e.g., target sequence-specific primer (“TSP”)), second oligonucleotide (e.g., locus-specific primer (“LSP”)), and third oligonucleotide (e.g., target site-specific probe) aligned with an exemplary target polynucleotide.

FIGS. 2A through 2F. Exemplary amplification of a target polynucleotide using exemplary target sequence-specific primer (TSP), and locus-specific primer (LSP), for detection of a KRAS G12R (34G>C) target polynucleotide, with (FIGS. 2A, 2B and 2C) and without (FIGS. 2D, 2E and 2F) an enrichment cycle. Samples of wild-type DNA (10 ng) or the spiked samples (10 ng wild-type DNA (Control DNA from CEPH Individual 1347-02; Thermo Fisher Scientific Cat. No. 403062; hereinafter referred to as “CEPH”) with 0.1% allelic variant DNA (e.g., mutant DNA) spiked in) were combined with 300 nM each primer, 250 nM probe, 1 mM dNTPs, 39 mM Tris pH 8, 2.55 mM MgCl₂, 30 mM KCl, 16 mM (NH₄)₂SO₄, 0.1 mg/mL BSA, 7% glycerol and 0.085 U/uL Platinum Taq to form a reaction mixture. Next, 10 μl aliquots of the mixtures were plated in 4 replicate wells of a 96-well plate. A qPCR reaction was performed in each well on a QuantStudio 5 F96 (Thermo Fisher Scientific, Waltham, Mass.). PCR reactions were performed with an enrichment phase (FIGS. 2A, 2B and 2C) or without an enrichment phase (FIGS. 2D, 2E and 2F).

FIGS. 3A through 3F. Exemplary amplification of target polynucleotide using exemplary target sequence-specific primer (TSP), and locus-specific primer (LSP), for the KRAS G12A (35G>C) target polynucleotide, with (FIGS. 3A, 3B and 3C) and without (FIGS. 3D, 3E and 3F) an enrichment cycle. Samples of wild-type DNA (10 ng) or the spiked samples (10 ng wild-type (CEPH) DNA with 0.1% allelic variant DNA (e.g., mutant DNA) spiked in) were combined with 300 nM each primer, 250 nM probe, 1 mM dNTPs, 39 mM Tris pH 8, 2.55 mM MgCl₂, 30 mM KCl, 16 mM (NH₄)₂SO₄, 0.1 mg/mL BSA, 7% glycerol and 0.085 U/uL Platinum Taq to form a reaction mixture. Next, 10 μl aliquots of the mixtures were plated in 4 replicate wells of a 96-well plate. A qPCR reaction was performed in each well on a QuantStudio 5 F96 (Thermo Fisher Scientific, Waltham, Mass.). PCR reactions were performed with an enrichment phase (FIGS. 3A, 3B and 3C) or without an enrichment phase (FIGS. 3D, 3E and 3F).

FIGS. 4A through 4F. Discrimination of wild-type (CEPH) DNA and wild-type (CEPH) DNA individually spiked with 0.1% of the following allelic variant (e.g., mutant) KRAS DNA: KRAS G12R (34G>C; Horizon Discovery Ltd. Cat. No. HD287) (FIG. 4A), KRAS G12A (35G>C; Horizon Discovery Ltd. Cat. No. HD265) (FIG. 4B), KRAS G12S (34G>A; Horizon Discovery Ltd. Cat. No. HD288) (FIG. 4C), KRAS G12C (34G>T; Horizon Discovery Ltd. Cat. No. HD269) (FIG. 4D), KRAS G12D (35G>A; Horizon Discovery Ltd. Cat. No. HD272) (FIG. 4E), or KRAS G12V (35G>T; Horizon Discovery Ltd. Cat. No. HD289) (FIG. 4F). PCR conditions were as described above for FIGS. 2 and 3, with enrichment.

FIGS. 5A and 5B. In FIG. 5A, discrimination between a mutant KRAS G13D (“G13D”) target polynucleotide and wild-type (CEPH) nucleic acid was observed in qPCR reactions including up to 60 mM KCl (potassium chloride) and up to 30 mM (NH₄)₂SO₄ (ammonium sulfate). In FIG. 5B, discrimination between a mutant KRAS G13D target polynucleotide and wild-type (CEPH) nucleic acid was not observed in qPCR reactions lacking KCl and (NH₄)₂SO₄ (ammonium sulfate). Wild-type DNA was spiked with 0.2% of mutant KRAS G13D target polynucleotide in both reactions.

FIGS. 6A through 6D. Effects of potassium chloride and ammonium sulfate on amplification and detection of target polynucleotides. 20 uL reaction mixtures containing 10 ng wild-type (CEPH) DNA; 300 nM each primer (TSP and LSP); 250 nM probe; 1 mm dNTPs; 0.085 U/uL Platinum Taq; 2.55 mM MgCl₂; 45 nM ROX passive reference; 39 mM Tris, pH 8; and 7% glycerol were prepared. Potassium chloride and ammonium sulfate were titrated into the reaction mixtures as described below: FIG. 6A: no KCl or ammonium sulfate (no discrimination); FIG. 6B: 30 mM ammonium sulfate, no potassium chloride (some discrimination); FIG. 6C: 30 mM KCl and 30 mM ammonium sulfate (sufficient discrimination); and, FIG. 6D: 60 mM KCl and 30 mM ammonium sulfate (suppressed reaction). The reactions with G13D mutant spike contained 20 pg KRAS G13D Reference Standard DNA (Horizon Discovery Ltd.; Cat. No. HD290) spiked into wild-type (CEPH) DNA at 0.2%. The reactions were amplified on a QuantStudio 7 instrument using the following thermal cycling protocol: 95° C. (3 min); 95° C. (3 sec),/64° C. (20 sec) for 19 cycles (enrichment phase); then 95° C. (3 sec)/60° C. (20 sec) for 40 cycles (amplification and detection phase).

FIGS. 7A and 7B. Testing of intermediate concentrations using qPCR conditions as described above in FIG. 6 (with 0.15% mutant DNA spiked into wild-type DNA), but including the indicated concentrations of KCl and ammonium sulfate. FIG. 7A included 45 mM KCl and 30 mM ammonium sulfate. FIG. 7B included 45 mM KCl and 22 mM ammonium sulfate.

FIG. 8. The effect of distance of the target variable nucleotide from the end of the third oligonucleotide (e.g., target site-specific probe). Experiments were carried out in 20 uL reactions containing: 1 mM dNTPs, 45 mM KCl, 22 mM ammonium sulfate, 0.085 U/uL Platinum Taq, 2.55 mM MgCl₂, 45 nM ROX passive reference, 39 mM Tris pH 8 and 7% glycerol, 300 nM of each primer (TSP and LSP), 250 nM of one of Probes 1, 2 or 3; 10 ng wild-type (CEPH) DNA; and 10 pg of EGFR L858R Reference Standard DNA (Horizon Discovery Ltd.; Cat. No. HD254). The illustrated data shows that the effective amplification and real-time detection was achieved with the target variant nucleotide located at 3 (Probe 1), 4 (Probe 2), or 5 (Probe 3) nucleotides from the 3′ end of the probe.

FIGS. 9A through 9F. Titration of mutant target DNA spiked into wild-type DNA and differentiation thereof. These experiments were performed in 20 uL reactions containing 10 ng wild-type (CEPH) DNA, 1 mM dNTPs, 45 mM KCl, 22 mM ammonium sulfate, 0.085 U/uL Platinum Taq, 2.55 mM MgCl₂, 45 nM ROX passive reference, 39 mM Tris pH 8 and 7% glycerol. A 50 fM solution of artificial mutant template containing the indicated point mutation was first diluted to 3000 copies/uL, then to 250 copies/uL, and then two-fold dilutions were performed down to 2 copies/uL. The reactions were amplified on a QuantStudio 7 instrument using the following thermal cycling protocol: 95° C. (3 min); 95° C. (3 sec)/64° C. (20 sec) for 19 cycles (enrichment phase); then 95° C. (3 sec)/60° C. (20 sec) for 40 cycles. The experiments were performed using the following amount and copies of primers and target polynucleotide: FIG. 9A included 300 nM of each primer and 250 copies of target polynucleotide; FIG. 9B included 300 nM of each primer and 16 copies of target polynucleotide; FIG. 9C included 300 nM of each primer and two (2) copies of target polynucleotide; FIG. 9D included 450 nM of each primer and 250 copies of target polynucleotide; FIG. 9E included 450 nM of each primer and 16 copies of target polynucleotide; and FIG. 9F included 450 nM of each primer and two (2) copies of target polynucleotide.

FIG. 10. Titration of mutant DNA into wild-type DNA. These experiments were performed, as described for FIGS. 9A-9F, using 20 uL reactions containing 10 ng wild-type (CEPH) DNA, 1 mM dNTPs, 45 mM KCl, 22 mM ammonium sulfate, 0.085 U/uL Platinum Taq, 2.55 mM MgCl₂, 45 nM ROX passive reference, 39 mM Tris pH 8 and 7% glycerol. A 50 fM solution of the artificial mutant template was first diluted to 3000 copies/uL, then to 250 copies/uL, and then two-fold dilutions were performed down to 2 copies/uL. The reactions were amplified on a QuantStudio 7 instrument using the following thermal cycling protocol: 95° C. (3 min); 95° C. (3 sec)/64° C. (20 sec) for 19 cycles (enrichment phase); then 95° C. (3 sec)/60° C. (20 sec) for 40 cycles. The amount of each primer and copies of variant allele (e.g., mutant) polynucleotides spiked into the reactions were as indicated. The left side y-axis illustrates the Cq for FAM. The right side axis illustrates the delta Cq for FAM-VIC.

FIG. 11. Dilution of the indicated number of copies 3000; 300; 30; and three (3) copies of target polynucleotide (NRAS Q61R) was spiked into wild-type DNA. In these experiments, 20 uL reaction mixtures containing 300 nM of each primer (TSP and LSP); 250 nM probe; 10 ng wild-type (CEPH) DNA; and the indicated amount of mutant DNA (Horizon Discovery Ltd.; Cat. No. HD574) @ less than 1 copy, 3 copies, 30 copies, 300 copies, or 3000 copies; 1 mM dNTPs; 45 mM KCl; 22 mM ammonium sulfate; 0.085 U/uL Platinum Taq; 2.55 mM MgCl₂; 45 nM ROX passive reference; 39 mM Tris, pH 8; and 7% glycerol were prepared. The data was exported in Excel format, the Cq values for replicate reactions averaged and the delta average Cq of targets in each condition determined and plotted (data not shown). The delta Cq was used as the quantification method. Thermal cycling conditions on QuantStudio 7 were: 95° C. (3 min); Enrichment phase-19 cycles of 95° C. (3 sec)/64° C. (20 sec); Amplification-40 cycles of 95° C. (3 sec)/60° C. (20 sec). “ntc”=no template control.

FIG. 12. Dilution of indicated numbers of copies (two, four, eight, 16, 31, 62, 125, or 250 copies) of target polynucleotide (NRAS Q61K) was spiked into wild-type DNA. In these experiments, 20 uL reaction mixtures containing 300 nM each primer (TSP and LSP); 250 nM probe; 10 ng wild-type (CEPH) DNA; and the indicated amount of mutant (Horizon Discovery Ltd.; Cat. No. HD351) @ 2, 4, 8, 16, 31, 62.5, 125, or 250 copies; 1 mM dNTPs; 45 mM KCl; 22 mM ammonium sulfate; 0.085 U/uL Platinum Taq; 2.55 mM MgCl₂; 45 nM ROX passive reference; 39 mM Tris, pH 8; and 7% glycerol were prepared. The data was exported in Excel format, the Cq values for replicate reactions averaged and the delta average Cq of targets in each condition determined and plotted (data not shown). The delta Cq was used a quantification method. Thermal cycling conditions on Quant Studio 7 were: 95° C. (3 min); 19 cycles of 95° C. (3 sec)/64° C. (20 sec); 40 cycles of 95° C. (3 sec)/60° C. (20 sec).

FIGS. 13A through 13C. Dilution of three (3) copies of target polynucleotide (KRAS G12R) into 20 ng (0.05% spike-in; FIG. 13A), 10 ng (0.1% spike-in; FIG. 13B), and 5 ng (0.2% spike-in; FIG. 13C) wild-type DNA. These experiments were carried out in reaction mixtures containing 300 nM of the KRAS forward and reverse primers; 100 nM RPPH1 forward and reverse primers; 250 nM KRAS-FAM probe; 150 nM RPPH1-VIC probe; 1 mM dNTPs; 45 mM KCl; 22 mM ammonium sulfate; 0.085 U/uL Platinum Taq; 2.55 mM MgCl₂; 45 nM ROX passive reference; 39 mM Tris pH 8; 7% glycerol. 10 pg KRAS G12R Reference Standard DNA (Horizon Discovery Ltd.; Cat. No. HD287); and the indicated amount of wild-type (CEPH) DNA were prepared. The reactions were run on a QuantStudio 5 using the following thermal cycling protocol: 95° C. (2 min); 95° C. (1 sec)/64° C. (20 sec) for 19 cycles (enrichment); then 95° C. (1 sec)/60° C. (20 sec) for 40 cycles.

FIG. 14. Use of a reverse KRAS G12D (35G>A) primer in amplification of target polynucleotide. These experiments were carried out in 20 uL reaction mixtures containing 10 ng of wild-type (CEPH) DNA; 300 nM of the indicated KRAS forward and reverse primers; 250 nM KRAS probe; a spike of 10 pg of mutant KRAS (G12D or G12S, as indicated) Reference Standard DNA (Horizon Discovery Ltd.; Cat. Nos. HD272 or HD288, respectively) were utilized; 1 mM dNTPs; 45 mM KCl; 22 mM ammonium sulfate; 0.085 U/uL Platinum Taq; 2.55 mM MgCl₂; 45 nM ROX passive reference; 39 mM Tris, pH 8; and 7% glycerol were prepared. Thermal cycling conditions were: 95° C. (3 min), 19 cycles of 95° C. (3 sec), 64° C. (20 sec) (enrichment); followed by 40 cycles of 95° C. (3 sec)/60° C. (20 sec).

FIG. 15. Use of reverse KRAS G12S (34G>A) primer in amplification of target polynucleotide, carried out as described for FIG. 14, but using the reverse primer for KRAS G12S (34G>A) instead.

FIGS. 16A through 16F. Amplification of target polynucleotide (0.1% mutant target polynucleotide) in the presence of wild-type DNA. FIG. 16A. EGFR20 T790M (2369C>T; Horizon Discovery Ltd.; Cat. No. HD258) target polynucleotide. FIG. 16B. EGFR19 (de1746-750; Horizon Discovery Ltd.; Cat. No. HD251) target polynucleotide. FIG. 16C. NRAS G12D (35G>A; Horizon Discovery Ltd.; Cat. No. HD745) target polynucleotide. FIG. 16D. BRAF V600E (1799T>A; Horizon Discovery Ltd.; Cat. No. HD238) target polynucleotide. FIG. 16E. NRAS G13D (38G>A; Horizon Discovery Ltd.; Cat. No. HD745) target polynucleotide. FIG. 16F. EGFR L861Q (2582T>A; Horizon Discovery Ltd.; Cat. No. HD257) target polynucleotide. Each of the experiments were carried out in 20 uL reaction mixtures containing 10 ng of wild-type (CEPH) DNA; 300 nM each of the first and second oligonucleotides (e.g., target sequence-specific primer (TSP) and locus-specific primer (LSP)); 250 nM probe; a spike of 10 pg of the corresponding mutant DNA listed above were utilized (Horizon Discovery Ltd.; Reference Standards); 1 mM dNTPs; 45 mM KCl; 22 mM ammonium sulfate; 0.085 U/uL Platinum Taq; 2.55 mM MgCl₂; 45 nM ROX passive reference; 39 mM Tris, pH 8; and 7% glycerol were prepared. The data was exported in Excel format, the Cq values for replicate reactions averaged and the delta average Cq of targets in each condition determined and plotted (data not shown). The delta Cq was used a quantification method. The thermal cycling conditions used were: 95° C. (3 min), 19 cycles of 95° C. (3 sec)/64° C. (20 sec) (enrichment); followed by 40 cycles of 95° C. (3 sec)/60° C. (20 sec).

FIGS. 17A and 17B. Amplification of target polynucleotides using probes of different lengths. FIG. 17A. NRAS Q61L (182A>T), 16 and 21 nucleotide probes. FIG. 17B. NRAS Q61H (183A>T), 15 and 20 nucleotide probes. These experiments were carried out in 20 uL reaction mixtures containing 300 nM each primer; 250 nM of the corresponding probe; 10 ng wild-type (CEPH) DNA; a mutant spike of 20 pg NRAS Q61L or Q61H Reference Standard DNA (Horizon Discovery Ltd.; Cat. No. HD412 or HD303, respectively) were included; 1 mM dNTPs; 45 mM KCl; 22 mM ammonium sulfate; 0.085 U/uL Platinum Taq; 2.55 mM MgCl₂; 45 nM ROX passive reference; 39 mM Tris, pH 8; and 7% glycerol were prepared. The reactions were run on a QuantStudio 5 using the following thermal protocol: 95° C. (2 min); 95° C. (1 sec)/64° C. (20 sec) for 19 cycles (enrichment); then 95° C. (1 sec)/60° C. (20 sec) for 40 cycles.

FIGS. 18A through 18C. Amplification of target polynucleotide (0.1% mutant target polynucleotide) in the presence of wild-type (CEPH) DNA. FIG. 18A. ESR1 E380Q (1138G>C) target polynucleotide. FIG. 18B. PIK3CA H1047R (3140A>G) target polynucleotide. FIG. 18C. TP53 H179Q (537T>A) target polynucleotide. Each of the experiments were carried out in 20 uL reaction mixtures containing 10 ng of wild-type (CEPH) DNA; 300 nM each of the first and second oligonucleotides (e.g., target sequence-specific primer (TSP) and locus-specific primer (LSP) for each of the indicated mutant targets and a RPPH1 control target); 250 nM probe for each of the indicated mutant targets and a RPPH1 control target; a spike of 10 pg of the corresponding mutant DNA listed above were utilized; 1 mM dNTPs; 45 mM KCl; 22 mM ammonium sulfate; 0.085 U/uL Platinum Taq; 2.55 mM MgCl₂; 45 nM ROX passive reference; 39 mM Tris, pH 8; and 7% glycerol were prepared. The data was exported in Excel format, the Cq values for replicate reactions averaged and the delta average Cq of targets in each reaction condition determined and plotted (data not shown). The delta Cq between the RPPH1 control target and each individual mutant target was used as a quantification method. The thermal cycling conditions used were: 95° C. (3 min), 19 cycles of 95° C. (3 sec)/64° C. (20 sec) (enrichment); followed by 40 cycles of 95° C. (3 sec)/60° C. (20 sec).

FIGS. 19A and 19B. Amplification of target polynucleotide (0.1% mutant target polynucleotide) in the presence of wild-type (CEPH) DNA. FIG. 19A. TP53 Y220C (659A>G) target polynucleotide. FIG. 19B. TP53 R249M (746G>T) target polynucleotide. Each of the experiments were carried out in 20 uL reaction mixtures containing 10 ng of wild-type (CEPH) DNA; 300 nM each of the first and second oligonucleotides (e.g., target sequence-specific primer (TSP) and locus-specific primer (LSP) for each of the indicated mutant targets and a RPPH1 control target); 250 nM probe for each of the indicated mutant targets and a RPPH1 control target; a spike of 10 pg of the corresponding mutant DNA listed above were utilized; 1 mM dNTPs; 45 mM KCl; 22 mM ammonium sulfate; 0.085 U/uL Platinum Taq; 2.55 mM MgCl₂; 45 nM ROX passive reference; 39 mM Tris, pH 8; and 7% glycerol were prepared. The data was exported in Excel format, the Cq values for replicate reactions averaged and the delta average Cq of targets in each reaction condition determined and plotted (data not shown). The delta Cq between the RPPH1 control target and each individual mutant target was used as a quantification method. The thermal cycling conditions used were: 95° C. (3 min), 19 cycles of 95° C. (3 sec)/64° C. (20 sec) (enrichment); followed by 40 cycles of 95° C. (3 sec)/60° C. (20 sec).

FIG. 20. Exemplary wild-type EGFR and BRAF sequences. Bolded nucleotides are representative of the bases which are mutated in the mutant polynucleotides described elsewhere herein.

FIG. 21. Exemplary wild-type KRAS and NRAS sequences. Bolded nucleotides are representative of the bases which are mutated in the mutant polynucleotides described elsewhere herein (ex., KRAS c.34G, c.35G, c.38G).

### Definitions

As used herein, the terms “minor allele” or “minor allelic variant” refer to a target polynucleotide present at a lower level in a sample as compared to an alternative allelic variant (e.g., an “abundant allele” such as a “major allele” and/or a “wild-type allele”). For instance, the minor allelic variant may be found at a frequency (e.g., have a minor allelic frequency (“MAF”) of) less than 1/10, 1/100, 1/1,000, 1/10,000, 1/100,000, 1/1,000,000, 1/10,000,000, 1/100,000,000 or 1/1,000,000,000 compared to another allelic variant for a given single nucleotide polymorphism (SNP) or gene. Alternatively, the rare allelic variant can be, for example, less than 2, 3, 4, 5, 6, 7, 8, 9, 10, 15, 20, 25, 50, 75,100,250, 500, 750, 1,000, 2,500, 5,000, 7,500, 10,000, 25,000, 50,000, 75,000, 100,000, 250,000, 500,000, 750,000, or 1,000,000 copies per 1, 10, 100, 1,000 micro liters of a sample or a reaction volume. In some embodiments, an allele present at a frequency of less than or equal to 1 in 1,000 copies compared to another allelic variant of a given SNP or gene can be referred to herein as a “rare allele,” “rare allelic variant,” “low abundance allele,” or “low abundance allelic variant.” Those of ordinary skill in the art will understand that minor alleles or minor allelic variants other than those explicitly defined herein will be applicable to this disclosure.

As used herein, the terms “abundant allele” refers to a target polynucleotide present at a higher level in a sample as compared to an alternative allelic variant. The abundant allele may also be referred to as a “major allele” and/or a “wild-type allele”. For instance, the abundant allele may be found at a frequency greater than 10×, 100×, 1,000×, 10,000×, 100,000×, 1,000,000×, 10,000,000×, 100,000,000× or 1,000,000,000× compared to an allelic variant for a given SNP or gene, and/or a major allele (or wild-type allele). Alternatively, the abundant allelic variant can be, for example, present at greater than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, 1,000,000 copies per 1, 10, 100, 1,000 micro liters of a sample or a reaction volume. Those of ordinary skill in the art will understand that abundant alleles other than those explicitly defined herein will be applicable to this disclosure.

### DETAILED DESCRIPTION OF THE INVENTION

This disclosure relates to reagents, mixtures, kits and methods for use in detecting one or more target polynucleotides (which may alternatively be referred to herein as target polynucleotides and/or target polynucleotide sequences), such as low abundance (or “rare”) target polynucleotides, that include at least one target variant nucleotide (e.g., mutated genetic variants and/or minor/particular allelic variants) using at least a first oligonucleotide that functions as a target sequence-specific primer (sometimes abbreviated “TSP”) having specificity for the at least one target variant nucleotide; at least one second oligonucleotide that functions as a primer with specificity for the target polynucleotide, but not the target variant nucleotide (sometimes referred to as a locus-specific primer (“LSP”)); and at least a third oligonucleotide that functions as a target site-specific probe with binding specificity for a target polynucleotide sequence including the at least one target variant nucleotide, and that can include a detectable property (e.g., a detectable label). In some illustrative embodiments, this disclosure relates to reagents, kits, and methods of using the first, second and third oligonucleotide in an amplification reaction that amplifies low abundance target polynucleotides from within a sample (e.g., mixture) comprising an abundance of alternative nucleic acid sequences (e.g., non-mutated, “wild-type”, or major allelic variants). In some embodiments, the low abundance target polynucleotides may be identified by detecting a change in a detectable property of a detectable oligonucleotide (e.g., a target-site specific probe such as the third oligonucleotide). Each mixture comprising a first, second and third oligonucleotide typically can include only one type of third oligonucleotide (e.g., a target site-specific probe) having binding specificity for a nucleic acid sequence comprising, or being complementary to, a particular target polynucleotide sequence that includes (or is complementary to) a particular target variant nucleotide. In some embodiments, however, a mixture can comprise different target sequence-specific primers, locus-specific primer, and/or target site-specific probes (e.g., such as in a multiplex reaction). In some embodiments, the oligonucleotides (i.e., the primers and probes), and/or mixtures thereof can be used to detect as little as, for instance, three or fewer copies of one or more low abundance target polynucleotide(s) (e.g., a rare target polynucleotide) in the presence of a more abundant “wild-type” nucleic acid (e.g., a non-mutated nucleic acid, or nucleic acid representing a major allele (e.g., “major allele” or “major allelic variant”)). In illustrative examples, a mixture may comprise, e.g., about 10 pg low abundance (or “rare”) target polynucleotides and about 10 ng of genomic DNA, or about 0.1% low abundance (or “rare”) target polynucleotides. Other embodiments, variations, and the like are contemplated herein and will be understood by those of ordinary skill in the art from this disclosure.

### Target Variant Nucleotide

The target variant nucleotide is a nucleotide residue within a target polynucleotide sequence that varies between different versions of a nucleic acid sequence (e.g., genes and/or coding sequences corresponding to particular mutants and/or alleles). In some embodiments, the target variant nucleotide is said to “correspond to”, be associated with, and/or be found within an allele (i.e., an allelic variant), where it represents a DNA sequence difference between two or more variants of a particular gene that, for the purposes of this disclosure, may be found within or associated with, a coding sequence of a gene, or a non-coding sequence of a gene. A target variant nucleotide can correspond to a major allele or a minor allele, and such minor allele can be found at a frequency that qualifies it as a low abundance allele or a rare allele. In some embodiments, the target variant nucleotide is part of a larger allelic sequence difference. In humans, the presence of certain alleles within an individual's genome can lead to variations such as, for example, eye color, but can also be associated with or correlate to particular disease conditions. In non-mammals such as plants, the presence of certain alleles in the genome can be used to, for instance, identify a particular plant species, subtype and/or genotype. A target variant nucleotide may also be present within a target polynucleotide as a result of genetic, stochastic (i.e., random), or other mutation. Exemplary mutations including a target variant nucleotide can include a nucleic acid sequence comprising a point or other mutation, which results from, e.g., a substitution, insertion or deletion of a “normal” nucleotide for another that results in an abnormal condition (e.g., disease). In some embodiments, the target variant nucleotide may correspond to an allelic variant that is present at a frequency less than 1/10, 1/100, 1/1,000, 1/10,000, 1/100,000, 1/1,000,000, 1/10,000,000, 1/100,000,000 or 1/1,000,000,000, and any fractional ranges in between, in a population of nucleic acid sequences. For instance, in some embodiments, the target variant nucleotide corresponds to the identity of a minor allelic sequence having a population frequency of less than about any of 1%, 0.1%, 0.01%, 0.001% or 0.0001% of a sample nucleic acid population (i.e., “population frequency” being referred to here in favor of a “sample population” because if the sample includes a minor allele, it might be 50% prevalent for a heterozygous individual). In certain embodiments, the target allele is a rare allele or low abundance allele.

In some embodiments, the target variant nucleotide can correspond to and/or occur at a position of a single nucleotide polymorphism (“SNP”). SNPs are heritable single-base pair variations that occur throughout an organism's genome. SNPs comprise the most common form of genetic variation, with some estimates of SNPs in a given human genome numbering more than 10 million. SNP genotyping plays a central role in characterizing individuals and populations, studying disease traits in humans and other organisms, and identifying genes responsible for advantageous crop traits. Thus, SNPs represent a common form of genetic variation between organisms, where a particular nucleotide is found in the genome of an individual organism that differs from that found at the corresponding position in another individual organism. SNPs can be linked SNPs (located outside a gene with no effect on protein production or function), coding SNPs (i.e., located within a coding region of a gene), or non-coding SNPs (i.e., located within a gene's regulatory sequences). In the human genome, SNPs typically occur at a frequency of about one in every 300 nucleotides (i.e., there can be about 10 million SNPs throughout the human genome), and some have been associated with disease (e.g., disease-linked SNPs). SNPs have also been associated with expression quantitative trait loci (eQTL), and some of these can be cell-type specific. SNPs associated with disease are particularly relevant to this disclosure. SNPs are also found in non-human organisms such as plants. In some embodiments, the target variant nucleotide can correspond to and/or occur at any such SNPs.

In some embodiments, the target variant nucleotide can correspond to a point mutation, which can also be considered an allelic variant herein. In humans, exemplary disease conditions that can result from one or more point mutations that could serve as a target variant nucleotide include, but are not limited to, e.g., cystic fibrosis (caused by the F508 mutation), cancer (e.g., to a tumor suppressor gene or certain cancer-associated kinases), neurofibromatosis (Neurofibromin 1 or 2 mutation), sickle-cell anemia ((3-globin mutation), Tay-Sachs disease (HEXA mutations), and colorblindness (e.g., X chromosome mutations). Exemplary mutations associated with cancer include, but are not limited to, mutations to Ras (e.g., a KRAS (e.g., in codon 12 and/or codon 13) or and/or a NRAS mutation), EGFR, Kit, pTEN, TP53 (also known as p53), PIK3CA, AKT1, and/or ESR1 (such as those listed in Table 1 and Table 2, and as described in more detail below). A target variant nucleotide may correspond to, match, or be associated with any such mutations. In some embodiments, then, a mutant allele can include a target variant nucleotide. In some embodiments, the mutant allele can be either a purine-to-purine single point or base mutation or a pyrimidine-to-pyrimidine single point or base mutation at the target variant nucleotide. In some embodiments, the mutant allele can be a stochastic mutation. In some embodiments, the target variant nucleotide can have an identity corresponding to a major allelic sequence or a minor allelic sequence. Methods provided herein can be used to detect and, optionally, quantify a major allele and a minor allele. Thus, the identity of a target variant nucleotide can be used with the oligonucleotides, mixtures, and methods disclosed herein to detect, differentiate, and, optionally, quantify a major allele and/or a minor allele. Similarly, the identity of a target variant nucleotide can be used with the oligonucleotides, mixtures, and methods disclosed herein to detect, differentiate, and, optionally, quantify a target polynucleotide related to an inherited or acquired disease and/or disorder.

TABLE 1
TP53 mutants

| | | Corresponding base |
|---|---|---|
| | Protein mutations | mutation (Coding DNA |
| No. | in TP53 gene | Sequence; “CDS”) |
| 1 | p.R248W | c.742C > T |
| 2 | p.R273H | c.818G > A |
| 3 | p.R282W | c.844C > T |
| 4 | p.Y220C | c.659A > G |
| 5 | p.R248Q | c.743G > A |
| 6 | p.M246fs | c.736delA |
| 7 | p.R196* | c.585_586CC > TT |
| 8 | p.P151A | c.451C > G |
| 9 | p.R273C | c.817C > T |
| 10 | p.H193R | c.578A > G |
| 11 | p.R248L | c.743G > T |
| 12 | p.P278S | c.832C > T |
| 13 | p.C238Y | c.713G > A |
| 14 | p.V216M | c.646G > A |
| 15 | p.G245C | c.733G > A |
| 16 | p.C238S | c.713G > C, c.712T > A |
| 17 | p.R249M | c.746G > T |
| 18 | p.V272M | c.814G > A |
| 19 | p.R158H | c.473G > A |
| 20 | p.R213* | c.637C > T |
| 21 | p.H179Q | c.537T > A |
| 22 | p.M246V | c.736A > G |
| 23 | p.I195T | c.584T > C |
| 24 | p.Y220H | c.658T > C |
| 25 | p.M237I | c.711G > A |
| 26 | p.W146* | c.437G > A. c.438G > A |
| 27 | p.V157I | c.469G > A |
| 28 | p.H179L | c.536A > T |
| 29 | p.C242F | c.725G > T |
| 30 | p.G244A | c.731G > C |
| 31 | p.P151T | c.451C > A |
| 32 | p.H179Y | c.535C > T |
| 33 | p.C275F | c.824G > T |
| 34 | p.G244S | 730G > A |
| 35 | p.C275F | c.824G > T |
| 36 | p.R213Q | c.638G > A |

*= denotes that the mutation leads to a termination codon

TABLE 2
Various other mutants

| | Protein mutations in | Corresponding base |
|---|---|---|
| No. | various genes | mutation (CDS) |
| 1 | KRAS p.G12R | c.34G > C |
| 2 | KRAS p.G12A | c.35G > C |
| 3 | KRAS p.G12S | c.34G > A |
| 4 | KRAS p.G12D | c.35G > A |
| 5 | KRAS p.G12C | c.34G > T |
| 6 | KRAS p.G12V | c.35G > T |
| 7 | KRAS p.G13D | c.38G > A |
| 8 | BRAF p.V600E | c.1799T > A |
| 9 | EGFR p.T790M | c.2369C > T |
| 10 | EGFR p.L858R | c.2573T > G |
| 11 | EGFR p.L861Q | c.2582T > A |
| 12 | EGFR19 | c.2235_2249del15 |
| | p.E746_A750del | |
| 13 | ESR1 p.D538G | c.1613A > G |
| 14 | ESR1 p.Y537S | c.1610A > C |
| 15 | ESR1 p.E380Q | c.1138G > A |
| 16 | PIK3CA p.H1047R | c.3140A > G |
| 17 | PIK3CA p.E726K | c.2176G > A |
| 18 | PIK3CA p.Q546P | c.1637A > C |
| 19 | PIK3CA p.Q546P | c.1637-1638AG > CC |
| 20 | PIK3CA p.E545K | c.1633G > A |
| 21 | PIK3CA p.E545G | c.1624A > G |
| 22 | PIK3CA p.E542K | c.1624G > A |
| 23 | PIK3CA p.E453K | c.1357G > A |
| 24 | PIK3CA p.N345K | c.1035T > A |
| 25 | AKT1 p.E17K | c.49G > A |
| 26 | EGFR p.C797S | c.2390G > C |
| 27 | EGFR p.C797S | c.2389T > A |

### First Oligonucleotide (Target Sequence-Specific Primer; “TSP”)

In certain aspects provided herein, the first oligonucleotide (e.g., the target sequence-specific primer “TSP”) typically corresponds to, is hybridizable to (e.g., is configured to hybridize to), or includes the complement of a target variant nucleotide, at the terminal nucleotide thereof, or within three nucleotides of the terminal nucleotide. While the target variant nucleotide is typically identified via a single variant nucleotide, it will be understood by those of ordinary skill in the art that this target variant nucleotide resides within a longer sequence of nucleotides (e.g., within a target polynucleotide such as within an allele or mutated gene), which is sometimes referred to herein as a first sequence in a first target polynucleotide. The first oligonucleotide (e.g., the TSP) therefore corresponds to, is hybridizable to (e.g., is configured to hybridize to), or includes a nucleotide sequence complementary to a target polynucleotide strand, and the first oligonucleotide includes, but is not limited to, and is typically terminated by, a nucleotide complementary to a target variant nucleotide sequence. The target polynucleotide strand may be either strand of a double-stranded nucleic acid. Complementarity of the first oligonucleotide (e.g., TSP) to the target polynucleotide sequence is therefore partially determined by the binding specificity defined by the first oligonucleotide (e.g., TSP) as a whole, but is, in particular, determined by the target variant nucleotide (or complement thereof) present at the terminus of the first oligonucleotide (e.g., TSP). Whether the first oligonucleotide (e.g., TSP) is a forward or reverse primer (e.g., in an amplification reaction), the target variant nucleotide is located at the 3′-end of the first oligonucleotide (e.g., TSP). In some embodiments, then, the first oligonucleotide is configured to hybridize to a first sequence in a first target polynucleotide strand, wherein the first sequence in the first target polynucleotide has a target variant nucleotide, and wherein the first oligonucleotide further has a nucleotide at its 3′-end that is positioned to hybridize to the target variant nucleotide. In some embodiments, the first oligonucleotide (e.g., TSP) can comprise between 10-30 nucleotides (e.g., any of 10, 11, 12, 13, 14, 15, 16, 17, 18, 19, 20, 21, 22, 23, 24, 25, 26, 27, 28, 29 or 30 nucleotides), or in illustrative embodiments, between 12 and 30 nucleotides, such as 15-22 nucleotides. Other forms and/or versions of the first oligonucleotide (e.g., TSP) are also contemplated herein as would be understood by those of ordinary skill in the art.

### Second Oligonucleotide (Locus-Specific Primer; “LSP”)

The second oligonucleotide (e.g., locus-specific primer “LSP”) typically exhibits binding specificity for the target polynucleotide (i.e. target polynucleotide) but not at the target variant nucleotide (or a complement thereof) position. The second oligonucleotide (e.g., LSP) and the first oligonucleotide (e.g., TSP) typically, but not necessarily, bind to different strands of a double-stranded target polynucleotide sequence. Thus, the second oligonucleotide (e.g., LSP) typically has binding specificity for a nucleotide sequence, sometimes referred to as a second sequence of the first polynucleotide strand, upstream or downstream of the first oligonucleotide (e.g., TSP). For example, where the target polynucleotide is a double-stranded nucleic acid, the second oligonucleotide (e.g., LSP) is typically configured to hybridize to a sequence complementary to, and in illustrative embodiments has identity to or is significantly identical to a nucleotide sequence positioned 3′ of, or in illustrative examples 5′ of, and on the strand to which the first oligonucleotide (e.g., TSP) binds (i.e., the second oligonucleotide (e.g., LSP)) binds the strand complementary to that which the first oligonucleotide (e.g., TSP) hybridizes, but at a site or position different from that to which the first oligonucleotide (e.g., TSP) binds). The second oligonucleotide (e.g., LSP) and third oligonucleotide (e.g., target site-specific probe) typically, but not necessarily, have binding specificity for different nucleotide sequences on the same strand of a double-stranded target polynucleotide sequence. In some embodiments, then, the second oligonucleotide has a sequence configured to hybridize to a sequence complementary to a second sequence of the first target polynucleotide strand, wherein the second sequence of the first target polynucleotide strand is located 5′ upstream from the first sequence of the first target polynucleotide strand. In some embodiments, the second oligonucleotide (e.g., LSP) can comprise between 10-30 nucleotides (e.g., any of 10, 11, 12, 13, 14, 15, 16, 17, 18, 19, 20, 21, 22, 23, 24, 25, 26, 27, 28, 29 or 30 nucleotides), or in illustrative embodiments, between 12-30, or between 15-25, nucleotides, such as 16-24 nucleotides. Other forms and/or versions of the second oligonucleotide (e.g., LSP) are also contemplated herein as would be understood by those of ordinary skill in the art.

### Third Oligonucleotide (Target Site-Specific Probe)

The third oligonucleotide (e.g., target site-specific probe) also has binding specificity at the target variant nucleotide position, or at a position corresponding to or complementary to a sequence comprising the target variant nucleotide, and typically is configured to hybridize to a sequence complementary to a third sequence of the first target polynucleotide strand, wherein the target variant nucleotide is between one and eight nucleotides from a 5′ terminus of the third oligonucleotide. However, typically the third oligonucleotide has a nucleotide identical to the target variant nucleotide but typically not at the terminal nucleotide of the third oligonucleotide. Thus, in some embodiments the nucleotide identical to the target variant nucleotide within the third oligonucleotide is instead positioned near, but not at the terminal nucleotide. For instance, the target variant nucleotide is typically positioned at least two, and/or within three to six, nucleotides of a terminal nucleotide of the third oligonucleotide (e.g., target site-specific probe). Thus, in some embodiments, the target variant nucleotide in the target site-specific probe (e.g., third oligonucleotide) is at least two, and/or within two, three, four, five or six nucleotides from a 3′ end thereof. In some illustrative embodiments, the target variant nucleotide in the target site-specific probe (e.g., third oligonucleotide) is near the middle of the same, for example within one to seven nucleotide residues (i.e., one, two, three, four, five, six or seven nucleotide residues) from the 3′ or 5′ end of the target site-specific probe (e.g., third oligonucleotide). In some embodiments, the target variant nucleotide is positioned within 3-5 nucleotide residues from the 3′ or 5′ end of the target site-specific probe (e.g., third oligonucleotide). In some preferred embodiments, the target variant nucleotide is positioned within 3-5 nucleotide residues from the 3′ end of the target site-specific probe (e.g., third oligonucleotide). In some preferred embodiments, the target variant nucleotide is positioned within 3-5 nucleotide residues from the 5′ end of the target site-specific probe (e.g., third oligonucleotide). In some embodiments, the target variant nucleotide in the target site-specific probe is positioned about 1-3 nucleotide residues from the nucleotide(s) at the middle position(s) thereof. In some embodiments, the target variant nucleotide in the target site-specific probe (e.g., third oligonucleotide) is at least two nucleotides from a 3′-end thereof. The target site-specific probe (e.g., third oligonucleotide) also typically includes nucleotide sequences that overlap with the first sequence of the first target polynucleotide to which the first oligonucleotide (e.g., TSP) is configured to hybridize (typically being complementary thereto), and also includes sequences that do not overlap the first sequence of the first target polynucleotide (e.g., in some embodiments, two to seven nucleotides). In some embodiments, the number of overlapping nucleotide residues between the target site-specific probe and the first sequence of the first target polynucleotide to which the first oligonucleotide (e.g., TSP) is configured to hybridize is between two and seven (i.e., two, three, four, five, six, or seven nucleotide residues). In some preferred embodiments, the number of overlapping nucleotide residues between the target site-specific probe and the first sequence of the first target polynucleotide to which the first oligonucleotide (e.g., TSP) is configured to hybridize is between three and five (i.e., three, four or five nucleotide residues). In some preferred embodiments, the number of overlapping nucleotide residues between the target site-specific probe and the first sequence of the first target polynucleotide to which the first oligonucleotide (e.g., TSP) is configured to hybridize is three nucleotides (e.g., 3 bases). In some embodiments, target-site specific probe (e.g., the third oligonucleotide) can be designed according to the methods and principles described in U.S. Pat. No. 6,727,356 (the disclosure of which is incorporated herein by reference in its entirety); and/or can be a hydrolysis or TagMan® probe (Thermo-Fisher (Foster City, Calif.)). The target site-specific probe (e.g., third oligonucleotide) and the first oligonucleotide (e.g., TSP) typically, but not necessarily, are hybridizable to different strands of a double-stranded target polynucleotide sequence. In some embodiments, the target site-specific probe (e.g., third oligonucleotide) is detectable (e.g., comprises a detectable property such as a detectable label) and has a sequence configured to hybridize to a sequence complementary to a third sequence of the first target polynucleotide strand, wherein the third sequence of the first target polynucleotide strand overlaps at least partially with the first sequence of the first target polynucleotide strand and the target variant nucleotide. Other forms and/or versions of the third oligonucleotide are also contemplated herein as would be understood by those of ordinary skill in the art.

In some embodiments, the target site-specific probe (e.g., third oligonucleotide) may be from 12 to 40 nucleotides in length (e.g., any of 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides), or illustrative embodiments from 10-25 nucleotides in length, such as 11-23 nucleotides. In some embodiments, the melting temperature (Tₘ) of the target site-specific probe (e.g., third oligonucleotide) is at least 5° C. and no more than 25° C. higher than the Tₘ of the first oligonucleotide (e.g., TSP). In some embodiments, the Tₘ of the target site-specific probe (e.g., third oligonucleotide) is at least 8° C. and no more than 12° C. higher than the Tₘ of the first oligonucleotide (e.g., TSP). In some embodiments, the Tₘ of the first oligonucleotide is within 5° C. of the Tₘ of the second oligonucleotide (e.g., LSP). In some embodiments, the Tₘ of the first oligonucleotide (e.g., TSP) is 45° C. to about 60° C., about 48° C. to about 58° C., or 48° C. to 58° C. and the Tₘ of the second oligonucleotide (e.g., the LSP) is 45° C. to about 60° C., but within about 5° C. of one another. As used herein, the Tₘ of an oligonucleotide (e.g., a primer or probe) refers to the temperature (in degrees Celsius) at which 50% of the oligonucleotides in a population of a single-stranded oligonucleotide are hybridized to their complementary sequence and 50% of the oligonucleotides in the population are not hybridized to the complementary sequence. The Tₘ of an oligonucleotide can be determined empirically by means of a melting curve or using other methods or formulas well-known in the art (e.g., as described by Maniatis, T., et al., in Molecular Cloning: A Laboratory Manual (Cold Spring Harbor Laboratory, Cold Spring Harbor, N.Y.: 1982) and elsewhere in the art.

The target site-specific probe (e.g., third oligonucleotide) also optionally, and in some embodiments preferably, includes at least one detectable property, such as may be provided by, for example, at least one detectable label (e.g., a fluorescent label), that provides a detectable signal upon amplification of the target polynucleotide sequence. Such a detectable label can be, and is preferably, on a first terminal nucleotide (e.g., 5′ terminal or 3′ terminal base or nucleotide residue) of the target site-specific probe (e.g., third oligonucleotide), but typically not on the same nucleotide residue as another moiety such as a minor groove binder (MGB). In some embodiments, the detectable label is located on a first terminal nucleotide of the target site-specific probe. In some embodiments, the detectable label is located on a 5′ terminal nucleotide of the target site-specific probe. In other embodiments, the detectable label is located internally, but near the 5′ end of the probe (e.g., within the 5′ portion of the probe). A change in a detectable property of the target site-specific probe (e.g., third oligonucleotide) upon amplification of the target polynucleotide sequence typically indicates that the low abundance target polynucleotide sequence is present within a sample being assayed (e.g., a tissue sample). Typically, but not necessarily, neither the first oligonucleotide (e.g., TSP) nor the second oligonucleotide (e.g., LSP) comprises a detectable property such as may be provided by, for example, a detectable label. The detectable property may be provided to the target site-specific probe (e.g., third oligonucleotide) via one or more detectable labels. Suitable, non-limiting, and exemplary detectable labels include, for instance, a DNA-binding dye, reporter dye, a fluorescent probe, 6-carboxyfluorescein (FAM™), tetrachlorofluorescin (TET™), 6-Carboxy-4′,5′-Dichloro-2′,7′-Dimethoxyfluorescein, Succinimidyl Ester (JOE′), VIC™, a sulfonate derivative of a fluorescein dye with SO₃ instead of the carboxylate group, a phosphoramidite form of fluorescein, a phosphoramidite form of CY5, a non-FRET label, a ferrocene reagent; ABY™; NED™; and JUN™; Fluor®488, AlexaFluor®532, AlexaFluor®546, AlexaFluor®594, AlexaFluor®647, AlexaFluor®660, TYE™′M 563, PIE′ m665, and TYE™705, among others. In some embodiments, the detectable label on the third oligonucleotide (e.g., target site-specific probe) is on a first terminal nucleotide residue thereof. In some embodiments, the target site-specific probe can be a hydrolysis probe. Thus, in certain embodiments, the third oligonucleotide is cleaved by the exonuclease activity of a polymerase during nucleic acid synthesis or polymerization (e.g., when the enzyme extends a primer into the region of the probe) and a fluorescently labeled nucleotide or nucleotide fragment is released and detected. Multiplex assays in which more than one of such detectable labels (e.g., two, three, four, five or more) may be present in a single reaction (e.g., mixture) are also contemplated herein. In some embodiments, the mixtures may also comprise at least one passive reference dye (e.g., ROX™, Mustang Purple™).

The target site-specific probe (e.g., third oligonucleotide) can also include a quenching moiety capable of quenching a signal from the detectable label (e.g., a fluorescent label) prior to amplification of the target polynucleotide). Such a quenching moiety is typically attached to a nucleotide at a position from which that it is capable of quenching a signal from the detectable label. Thus, the quencher and label are preferably positioned at any length of nucleotides apart (and any length from the ends of the probe) so long as the two are not constrained by their position relative to one another, and are capable of coming into proximity to one another such that the signal from the detectable label is quenched (e.g., suppressed) when the probe is not hybridized to a complementary strand. For instance, in some embodiments, the detectable label attached to the target site-specific probe at the 5′ end thereof, and the quenching moiety is attached to the target site-specific probe at the 3′ end thereof. In some embodiments, the quenching moiety is on or near the 3′-end of the third oligonucleotide and the quenching moiety is on or near the 5′-end. In some embodiments, the quenching moiety is on a second terminal nucleotide of the target site-specific probe (e.g., third oligonucleotide) such as where the detectable label is on a first terminal nucleotide thereof. In some embodiments, then, the quenching moiety is capable of quenching a signal from the detectable label. Suitable, non-limiting, and exemplary quenchers include, for instance, tetramethylrhodamine (TAMRA), a non-fluorescent quencher (NFQ), Black Hole Quenchers, Iowa Black, QSY, QSY7, QSY21, NFQ, Dabsyl, and/or Dabsyl sulfonate/carboxylate Quenchers, among others. In some illustrative embodiments where the third oligonucleotide includes both a detectable label and a quenching moiety, the third oligonucleotide is a hydrolysis probe.

In some embodiments, the target site-specific probe (e.g., the third oligonucleotide) can be non-extendable and, to this point, can include a non-extendable blocker moiety such as a dideoxynucleotide (e.g., 2′3′-ddX) (wherein X can be C, A, G, or T)), a spacer such as a three carbon linker (C3), inverted dT, modified non-extendable primer blocker (NEBP; AS-NEBP-PCR (Wang, et al. J. Mol. Diagn. 15(1): 62-9 (2013)), or a minor groove binder (MGB; can be referred to as “MGB,” “MGB group,” “MGB compound,” or “MBG moiety”). In illustrative embodiments, the non-extendable blocker moiety is positioned at the 3′-terminal nucleotide residue of the third oligonucleotide. In some embodiments, then, the non-extendable blocker moiety may be a MGB. Oligonucleotides conjugated to MGB moieties form extremely stable duplexes with single-stranded and double-stranded DNA targets, thus allowing shorter probes to be used for hybridization based assays. In comparison to unmodified oligonucleotides or probes, MGB probes have higher melting temperatures (Tm) and increased specificity, especially when a mismatch is near the MGB region of the hybridized duplex. (See, e.g., Kutyavin, et al. Nucleic Acids Research, 2000, Vol. 28, No. 2: 655-661.) This means that MGB probes can be significantly shorter than traditional probes, providing better sequence discrimination and flexibility to accommodate more targets. Generally speaking, MGBs have a crescent shape three-dimensional structure and a strong preference for A-T (adenine and thymine)-rich regions of the B form of double stranded DNA. Nevertheless, MGB compounds which would show preference to C-G (cytosine and guanine)-rich regions are may also be useful as described herein. Some MGBs are capable of binding within the minor groove of double stranded DNA with an association constant of 10³M⁻¹ or greater. Such binding can be detected by well-established spectrophotometric methods such as ultraviolet (UV), nuclear magnetic resonance (NMR) spectroscopy, and/or gel electrophoresis. Shifts in UV spectra upon binding of a minor groove binder molecule and NMR spectroscopy utilizing the “Nuclear Overhauser” (NOSEY) effect are particularly well known and useful techniques for this purpose. Gel electrophoresis detects binding of an MGB to double stranded DNA or fragment thereof, because upon such binding the mobility of the double stranded DNA changes. A variety of suitable minor groove binders have been described in the literature (see, e.g., Kutyavin, et al. U.S. Pat. No. 5,801,155; Wemmer, D. E., and Dervan P. B., Current Opinion in Structural Biology, 7:355-361 (1997); Walker, W. L., Kopka, J. L. and Goodsell, D. S., Biopolymers, 44:323-334 (1997); Zimmer, C. & Wahnert, U. Prog. Biophys. Molec. Bio. 47:31-112 (1986) and/or Reddy, B. S. P., Dondhi, S. M., and Lown, J. W., Pharmacol. Therap., 84:1-111 (1999)). A preferred MGB in accordance with the present disclosure is DPI₃. Synthesis methods and/or sources for such MGBs are also well known in the art. (See, e.g., U.S. Pat. Nos. 5,801,155; 6,492,346; 6,084,102; and 6,727,356, the disclosures of which are incorporated herein by reference in their entireties.) When conjugated to the 3′ end of an oligonucleotide, an MGB group can function as a non-extendable blocker moiety. In some embodiments, the third oligonucleotide (e.g., target site-specific probe) may comprise a MGB moiety at its 3′- and/or 5′-end. In some embodiments, the MGB is positioned at the 3′-end (e.g., the 3′-terminal nucleotide residue) of the target site-specific probe (e.g., third oligonucleotide) or at the second or third nucleotide from a 3′-terminal nucleotide thereof. In some embodiments, the MGB moiety can be covalently attached to a quencher moiety. Thus, in some embodiments, a target site-specific probe (e.g., the third oligonucleotide) is typically detectable (e.g., comprises one or more detectable labels), comprises a quencher, and can also comprise a blocker moiety, such as MGB. As discussed above, in those instances in which a target site-specific probe (e.g., the third oligonucleotide) comprises one or more detectable labels and also comprises a quencher, the detectable label and quencher are typically positioned on opposite ends of the probe.

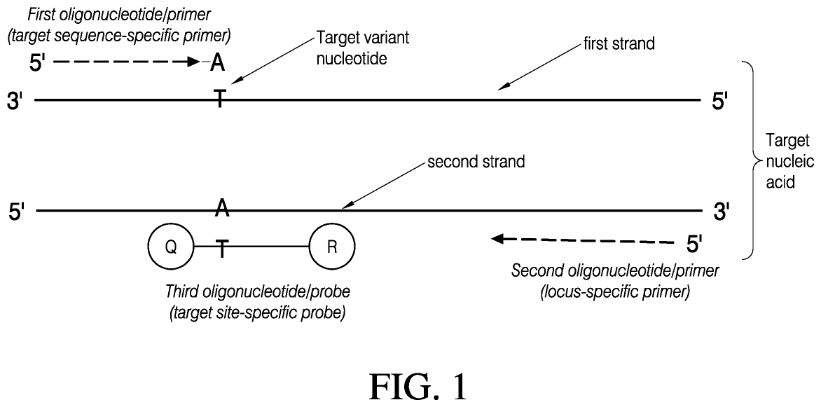

An embodiment of a mixture of at least one first oligonucleotide (e.g., TSP; a first primer), and at least one second oligonucleotide (e.g., LSP; a second primer), and at least one third oligonucleotide (e.g., a detectable target site-specific probe, a first probe), in the context of a double-stranded target polynucleotide (comprising “forward” and “reverse” strands) is illustrated in FIG. 1. In this illustration, the first oligonucleotide (e.g., TSP) is a “forward primer” complementary to the forward strand of the target polynucleotide and includes at its 3′ terminus a complement to the target variant nucleotide (e.g. “T”) present in that top strand. The third oligonucleotide (e.g., target site-specific probe) illustrated in FIG. 1 is complementary to the corresponding reverse strand of the target polynucleotide (i.e., has identity to a portion of the target polynucleotide forward strand) including a sequence comprising the target variant nucleotide. In some illustrative embodiments, this target site-specific probe also includes nucleotide residues that are complementary to and overlap the sequence to which the first oligonucleotide (e.g., TSP) binds as well as nucleotide residues that do not overlap the sequence to which the first oligonucleotide (e.g., TSP) binds. For instance, the embodiment illustrated in FIG. 1 includes a target site-specific probe having a 3′ sequence (or portion) overlapping the sequence of the target polynucleotide to which the target-specific primer (TSP) binds (i.e., the target site) and a 5′ sequence (or portion) that does not overlap the target site. In some embodiments, such as where a TSP is complementary to the “reverse” strand of the target polynucleotide, the target site-specific probe (e.g., first oligonucleotide) may include overlapping sequence at its 5′ end. In some embodiments, for example, the overlapping sequence may span two, three, four, five, six, or seven nucleotide residues. A second oligonucleotide (e.g., LSP) complementary to the reverse strand of the target polynucleotide 5′ of the target variant nucleotide (with reference to the forward strand of FIG. 1) is also illustrated. The target site-specific probe (e.g., third oligonucleotide) illustrated in FIG. 1 also includes a detectable label at its 5′ end (“R”) and a quencher at its 3′ end (“Q”). This combination of first oligonucleotide (e.g., TSP; the forward primer) and second oligonucleotide (e.g., LSP; the reverse primer) may then be used to amplify the target polynucleotide, where amplification of the target polynucleotide is determined by detecting release of the detectable label from the target site-specific probe (e.g., third oligonucleotide, the first probe, which in some embodiments may be a hydrolysis probe, such as a TaqMan′ probe). The first, second and third oligonucleotides, or various combinations thereof (e.g., first and second oligonucleotides, first and third oligonucleotides, second and third oligonucleotides) may be combined in the same or different amounts (e.g., equimolar or not equimolar) to provide a mixture(s) for use in carrying out the methods described herein.

Thus, in some embodiments, this disclosure provides compositions and/or mixtures comprising: a) a first oligonucleotide complementary to a first sequence in a first target polynucleotide strand, wherein: the first sequence comprises a target variant nucleotide; and, the first oligonucleotide comprises a nucleotide at its 3′-end that is complementary to the target variant nucleotide; b) a second oligonucleotide containing a sequence configured to hybridize to a sequence complementary (e.g., has identity to) to a second sequence located upstream or downstream of the first sequence on the first target polynucleotide strand; and, c) a (optionally detectable) third oligonucleotide (e.g., comprising a detectable label, and optionally a quencher and/or non-extendable blocker moiety) having a sequence configured to hybridize to a sequence complementary to a third sequence of the first target polynucleotide strand, wherein the third sequence shares identity with and at least partially overlaps the first sequence and includes the target variant nucleotide; as well as kits containing and methods for using the same. In some embodiments, the first and second oligonucleotides are extendable. In some embodiments, the first and second oligonucleotides are primers. In some embodiments, the third oligonucleotide can be non-extendable, can be a probe (e.g., a target site-specific probe) that can comprise a detectable label (e.g., as described above) and/or a quenching moiety (e.g., as described above), can comprise a minor groove binder (MGB) moiety (e.g., as described above), and/or is preferably distinguishable from any other oligonucleotide in the mixture that functions as a probe (e.g., a second, third, or fourth oligonucleotide probe, if present). In some embodiments, the first, second, and/or third oligonucleotide comprise between 10-40 nucleotides. In some embodiments, this disclosure provides methods for using the same and kits that can include instructions for using and/or storing such reagents and/or for carrying out such methods.

And in some embodiments, this disclosure provides mixtures comprising: a) a first oligonucleotide configured to hybridize to a first sequence in a first target polynucleotide strand, wherein the first sequence has a target variant nucleotide, and wherein the first oligonucleotide further has a nucleotide residue at its 3′-end complementary to the target variant nucleotide; b) a second oligonucleotide having a sequence configured to hybridize with a second sequence of the first target polynucleotide strand, wherein the second sequence is located upstream or downstream from the first sequence on the first target polynucleotide strand; and, c) a (optionally detectable) third oligonucleotide (e.g., target site-specific probe) (e.g., optionally comprising a detectable label, quencher and/or non-extendable blocker moiety), and a sequence having a sequence configured to hybridize with a third sequence of the first target polynucleotide strand, wherein the third sequence of the first target polynucleotide strand overlaps at least partially with the first sequence of the first target polynucleotide strand and includes a sequence comprising the target variant nucleotide; as well as methods for using the same and kits that can include instructions for using and/or storing such reagents and/or for carrying out such methods.

In some embodiments, this disclosure provides compositions and/or mixtures comprising: a) a first oligonucleotide configured to hybridize to a first sequence (A) present within a first target polynucleotide strand, wherein the first sequence includes a target variant nucleotide residue (“first variant nucleotide”), and wherein the first oligonucleotide further has a nucleotide at its 3′-end that is positioned to hybridize to the first variant nucleotide; b) a second oligonucleotide configured to hybridize to a second sequence (B), where the second sequence is complementary to a third sequence (C), the third sequence being present within the first target polynucleotide strand, wherein the third sequence (C) is located 5′ upstream from the first sequence (A) of the first target polynucleotide strand; and, c) a third oligonucleotide configured to hybridize to a fourth sequence (D) complementary to a fifth sequence (E), the fifth sequence present in the first target polynucleotide strand, wherein the fifth sequence (E) overlaps at least partially with the first sequence (A) in the first target polynucleotide strand and includes the first target variant nucleotide. In some embodiments, this disclosure provides methods for using the same and kits that can include instructions for using and/or storing such reagents and/or for carrying out such methods. In some embodiments, the first and second oligonucleotides are extendable. In some embodiments, the first and second oligonucleotides are primers. In some embodiments, the third oligonucleotide can be non-extendable, can be a probe (e.g., a target site-specific probe) that can comprise a detectable label (e.g., as described above) and/or a quenching moiety (e.g., as described above), can comprise a minor groove binder (MGB) moiety (e.g., as described above), and/or is preferably distinguishable from any other oligonucleotide in the mixture that functions as a probe (e.g., a second, third, or fourth oligonucleotide probe, if present). In some embodiments, the first, second, and/or third oligonucleotide comprise between 10-40 nucleotides. In some embodiments, this disclosure provides methods for using the same and kits that can include instructions for using and/or storing such reagents and/or for carrying out such methods.

Fourth, Fifth, and/or Sixth Oligonucleotides

In some embodiments, this disclosure provides compositions and/or mixtures (in some embodiments also including those described above having a first, second and third oligonucleotides, and in some embodiments not including those described above having such first, second and third oligonucleotides) that comprise: a) a fourth oligonucleotide having a sequence configured to hybridize to a first sequence in a second target polynucleotide strand, wherein the first sequence in the second target polynucleotide comprises a second target variant nucleotide, and wherein the fourth oligonucleotide further comprises a nucleotide at its 3′-end that is positioned to hybridize to the second target variant nucleotide; b) a fifth oligonucleotide comprising a sequence configured to hybridize to a sequence complementary to a second sequence of the second target polynucleotide strand, wherein the second sequence of the second target polynucleotide strand is located 3′ downstream from the first sequence on the second target polynucleotide strand; and, c) a sixth oligonucleotide configured to hybridize to a sequence complementary to a third sequence of the second target polynucleotide strand, wherein the third sequence of the second target polynucleotide strand overlaps at least partially with the first sequence on the second target polynucleotide strand and comprises the second target variant nucleotide (e.g., functioning as a probe by, e.g., comprising a detectable label; and optionally a quencher and/or non-extendable blocker moiety) comprising a sequence having identity to a third sequence of the second target polynucleotide strand, wherein the third sequence of the second target polynucleotide strand overlaps at least partially with the first sequence on the second target polynucleotide strand and comprises the second target variant nucleotide. In some embodiments, the fourth and fifth oligonucleotides are extendable. In some embodiments, the fourth and fifth oligonucleotides are primers. In some embodiments, the sixth oligonucleotide can be non-extendable, can be a probe (e.g., a target site-specific probe) that can comprise a detectable label (e.g., as described above) and/or a quenching moiety (e.g., as described above), can comprise a minor groove binder (MGB) moiety (e.g., as described above), and/or is preferably distinguishable from any other oligonucleotide in the mixture that functions as a probe (e.g., the third oligonucleotide, if present). In some embodiments, the fourth, fifth, and/or sixth oligonucleotide comprise between 10-40 nucleotides. In some embodiments, this disclosure provides methods for using the same and kits that can include instructions for using and/or storing such reagents and/or for carrying out such methods.

In some embodiments, this disclosure provides compositions and/or mixtures that (in some embodiments also including those described above having a first, second and third oligonucleotides, and in some embodiments not including those described above having such first, second and third oligonucleotides), comprise: a) a fourth oligonucleotide comprising a sequence that is configured to hybridize to a sequence complementary to the first oligonucleotide, wherein the fourth oligonucleotide is configured to substantially hybridize to the first sequence and comprises at its 3′ end a different nucleotide than the complement of the target variant nucleotide; and, b) a (optionally detectable) fifth oligonucleotide configured to hybridize to a sequence complementary to the third oligonucleotide, wherein the fifth oligonucleotide comprises a nucleotide at the position corresponding to the target variant nucleotide that is not the same or complementary thereto. In some embodiments, the fifth oligonucleotide can be non-extendable, can be a probe (e.g., a target site-specific probe), can comprise a detectable label (e.g., as described above), a quenching moiety (e.g., as described above), and/or a minor groove binder (MGB) moiety (e.g., as described above), and is distinguishable from the detectable property (e.g., detectable label; and optionally a quencher and/or non-extendable blocker moiety) of from any other oligonucleotide in the mixture that functions as a probe (e.g., the third oligonucleotide, if present). In some embodiments, the fourth, fifth, and/or sixth oligonucleotide comprise between 10-40 nucleotides. In some embodiments, this disclosure provides methods for using the same and kits that can include instructions for using and/or storing such reagents and/or for carrying out such methods.

### Type of Oligonucleotides

In some embodiments, the oligonucleotides disclosed herein, especially those functioning as a probe (e.g., the third oligonucleotide, target site-specific probe), can comprise one or more modified bases in addition to the naturally occurring bases adenine, cytosine, guanine, thymine and uracil. In some embodiments, the modified base(s) may increase the difference in the Tₘ between matched and mismatched target sequences and/or decrease mismatch priming efficiency, thereby improving not only assay specificity, but also selectivity. Modified bases can be those that differ from the naturally-occurring bases by addition or deletion of one or more functional groups, differences in the heterocyclic ring structure (i.e., substitution of carbon for a heteroatom, or vice versa), and/or attachment of one or more linker arm structures to the base. Such modified base(s) may include, for example, 8-Aza-7-deaza-dA (ppA), 8-Aza-7-deaza-dG (ppG), locked nucleic acid (LNA) or 2′-0,4′-C-ethylene nucleic acid (ENA) bases. Other examples of modified bases include, but are not limited to, the general class of base analogues 7-deazapurines and their derivatives and pyrazolopyrimidines and their derivatives (e.g., as described in PCT WO 90/14353). These base analogues, when present in an oligonucleotide, can strengthen hybridization and improve mismatch discrimination. All tautomeric forms of naturally occurring bases, modified bases and base analogues can be included. Modified internucleotide linkages can also be present in the oligonucleotides described herein. Such modified linkages include, but are not limited to, peptide, phosphate, phosphodiester, phosphotriester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as probes and/or primers, will be apparent to those of skill in the art. In addition, in some embodiments, the nucleotide units which are incorporated into the oligonucleotides acting as a probe (e.g., the third and/or sixth oligonucleotide, target site-specific probe), e.g., one comprising an MGB moiety, can have a cross-linking function (an alkylating agent) covalently bound to one or more of the bases, through a linking arm. Similarly, modified sugars or sugar analogues can be present in one or more of the nucleotide subunits of an oligonucleotide disclosed herein. Sugar modifications include, but are not limited to, attachment of substituents to the 2′, 3′ and/or 4′ carbon atom of the sugar, different epimeric forms of the sugar, differences in the alpha- or beta-configuration of the glycosidic bond, and other anomeric changes. Sugar moieties include, but are not limited to, pentose, deoxypentose, hexose, deoxyhexose, ribose, deoxyribose, glucose, arabinose, pentofuranose, xylose, lyxose, and cyclopentyl. In some embodiments, the sugar or glycoside portion of some embodiments of oligonucleotides acting as a probe (e.g., the third and/or sixth oligonucleotide, target site-specific probe), e.g., one comprising an MGB moiety, can comprise deoxyribose, ribose, 2-fiuororibose, 2-0 alkyl or alkenylribose where the alkyl group may have 1 to 6 carbons and the alkenyl group 2 to 6 carbons. In some embodiments, the naturally occurring nucleotides and in the herein described modifications and analogs the deoxyribose or ribose moiety can form a furanose ring, and the purine bases can be attached to the sugar moiety via the 9-position, the pyrimidines via the I-position, and the pyrazolopyrimidines via the I-position. And in some embodiments, especially in the oligonucleotides acting as a probe (e.g., the third and/or sixth oligonucleotide, target site-specific probe), the nucleotide units of the oligonucleotides can be interconnected by a “phosphate” backbone, as is well known in the art and/or can include, in addition to the “natural” phosphodiester linkages, phosphorothiotes and methylphosphonates. Other types of oligonucleotides or modified bases are also contemplated herein as would be understood by those of ordinary skill in the art.

### Oligonucleotide Sets

In some embodiments, this disclosure provides compositions and/or mixtures comprising a plurality of oligonucleotide sets, wherein each oligonucleotide set comprises: a) a first oligonucleotide (e.g., a TSP) that corresponds to, is hybridizable to (e.g., is configured to hybridize to), or includes a nucleotide sequence complementary to a target polynucleotide strand, the first oligonucleotide including, but not being limited to, and typically terminated by, a nucleotide complementary to a target variant nucleotide sequence, wherein the first oligonucleotide further comprises a nucleotide at its 3′-end that is positioned to hybridize to the target variant nucleotide); b) a second oligonucleotide (e.g., a LSP) comprising a sequence configured to hybridize to a sequence that is complementary to a second sequence to a second sequence of the target polynucleotide strand, wherein the second sequence is located upstream or downstream (e.g., 5′ upstream) from the first sequence; and, c) a (optionally detectable) third oligonucleotide (e.g., a target site-specific probe) comprising a sequence configured to hybridize to a sequence that is complementary to a third sequence of the target polynucleotide strand, wherein the third sequence overlaps at least partially with the first sequence and comprises the target variant nucleotide; wherein the first oligonucleotide of each set is configured to hybridize to a different first sequence; wherein the third oligonucleotide of each set has identity to a different third sequence; and, wherein the third oligonucleotide of each set comprises a different and distinguishable detectable property (e.g., different detectable labels). Each set of oligonucleotides typically includes only one detectable third oligonucleotide (e.g., a target site-specific probe) having binding specificity for a nucleic acid sequence comprising, or being complementary to, a target polynucleotide sequence including (or being complementary to) the target variant nucleotide. Thus, in some embodiments, the compositions and/or mixtures disclosed herein may comprise a plurality of oligonucleotide sets, wherein each oligonucleotide set comprises: a) a first oligonucleotide (e.g., a TSP) configured to hybridize to a first sequence in a target polynucleotide strand, wherein the first sequence comprises a target variant nucleotide, and wherein the first oligonucleotide further comprises a nucleotide at its 3′-end that is positioned to hybridize to the target variant nucleotide; b) a second oligonucleotide (e.g., a LSP) comprising a sequence configured to hybridize to a sequence that is complementary to a second sequence to a second sequence of the target polynucleotide strand, wherein the second sequence is located 5′ upstream from the first sequence; and, c) a third oligonucleotide (e.g., target site-specific probe) comprising both a detectable label and a sequence configured to hybridize to a sequence that is complementary to a third sequence of the target polynucleotide strand, wherein the third sequence overlaps at least partially with the first sequence and comprises the target variant nucleotide (or a complement thereof); wherein the first oligonucleotide of each set is configured to hybridize to a different first sequence, the third oligonucleotide of each set has identity to a different third sequence, and the third oligonucleotide of each set comprises a different and distinguishable detectable label. In some embodiments then, each set of oligonucleotides comprises a first oligonucleotide (e.g., a TSP as described above), a second oligonucleotide (e.g., a LSP as described above), and a detectable third oligonucleotide (e.g., a target site-specific probe as described above), where each third oligonucleotide of each set comprises a different detectable property such that the amplification of a first target polynucleotide sequence to which a first third oligonucleotide of a set binds can be distinguished from the amplification of any other target polynucleotide sequence to which any other third oligonucleotide of another set binds. Other embodiments are also contemplated by this disclosure as would be understood by those of ordinary skill in the art.

### PCR Reaction Mixtures

In certain aspects, the compositions and/or mixtures provided herein include one or more components known in the art as components of PCR reaction mixtures in addition to a first oligonucleotide (e.g. TSP), a second oligonucleotide (e.g. LSP), and a third oligonucleotide (e.g. target site-specific probe), or oligonucleotide sets thereof, as provided herein. The first oligonucleotide (e.g., TSP), second oligonucleotide (e.g., LSP), and third oligonucleotide (e.g., target site-specific probe), in certain embodiments, are each present at the same or different concentrations that can be between 0.05 uM and 1 uM, and in illustrative embodiments between 0.15 uM and 1 uM (e.g., about 250 nM, about 300 nM, about 400 nM, 450 nM, about 500 nM, about 550 nM, about 600 nM, about 650 nM, about 700 nM, about 750 nM, about 800 nM, about 850 nM, or about 900 nm, and in preferred embodiments about 300 nM or about 450 nM. In illustrative embodiments, the first (e.g., TSP) and second (e.g., LSP) oligonucleotides can each be present at a concentration of about 0.15 uM to about 0.45 uM, preferably about 0.15 uM, 0.30 uM, or 0.45 uM; and the third oligonucleotide (e.g. target site-specific probe) can be present at a concentration of about 0.25 uM. In illustrative embodiments such mixtures include components that result in amplification of a target when subjected to PCR thermocycling conditions. Such PCR reaction mixture components can include a source of free nucleotides, such as dNTPs, one or more PCR buffers, one or more thermostable polymerases, and Mg²⁺ at concentrations which allow for PCR or are used in such reaction mixtures. For example, Mg²⁺ can be present at 0.5-4 mM (preferably 2.55 mM), for example as MgSO₄ or MgCl, and dNTPs at equal concentrations for all 4 nucleotides at between 0.1 and 5 mM each, for example 2 mM each, or preferably 1 mM. Furthermore, in illustrative embodiments, such reaction mixtures include a target polynucleotide, which can act as a template polynucleotide in a PCR reaction. In some reaction mixtures provided herein, target polynucleotides are present at between 0.1 and 1 ug/ul or between 0.01 to 1 ng/nl. Thermostable polymerase(s) can be present at known activities for PCR reactions, such as for example between 0.01 units and 0.1 units per ul of reaction mixture. PCR buffers are known in the art and can be used at 0.5-2×, for example, at 1× concentrations. Furthermore, additional components such as potassium phosphate and ammonium sulfate can be present, as discussed in more detail herein. Additional components, such as but not limited to deoxynucleoside triphostphase (dNTPs), albumin such as bovine serum albumin (BSA; e.g., 10-100 μg/ml), buffer (e.g., Tris-HCl, pH between about 8 to 9.5), gelatin (such as fish and/or human gelatin; e.g., 0.01%), formamide (e.g., 1.25-10%), glycerol (e.g., 5-20%), polyethylene glycol (e.g., 5-15%), nonionic detergent(s) (e.g., Tween 20, Triton X-100; e.g., 0.05-1%), N-N-N-trimethylglycine (betaine; e.g., 1-3M), dimethylsulfoxide (DMSO; e.g., 1-10%), tetramethyl ammonium chloride (TMAC), and/or betaine, and/or combinations thereof, among other components, can also be present in the reaction mixtures provided herein.

### Potassium Phosphate and Ammonium Sulfate

The inventors/applicants surprisingly found that additional components increase the efficiency of target polynucleotide sequence amplification when included in effective amounts in the reactions and/or methods described herein. In some embodiments, then, the mixtures may comprise such additional components to increase the efficiency of target polynucleotide sequence amplification. In some embodiments, such additional components include effective amounts of potassium chloride and/or ammonium sulfate. The potassium chloride (KCl) and/or ammonium sulfate ((NH₄)₂SO₄) are included in amplification reactions in an “effective amount”, i.e., an amount of potassium chloride and/or ammonium sulfate that improves amplification of a target variant nucleotide over a more abundant nucleotide at the target nucleotide position in an amplification reaction (e.g., as shown in the Examples) when compared to amplification reactions that do not comprise said amount of potassium chloride and/or ammonium sulfate. Thus, the mixture may comprise a concentration of potassium chloride and/or ammonium sulfate that improves the differentiation of a target polynucleotide from a more abundant wild-type nucleic acid as determined for example by the Cq following an amplification reaction as compared to the Cq following an amplification reaction lacking said concentration of potassium chloride and/or ammonium sulfate. In some embodiments, the mixtures comprise an effective concentration of a combination of potassium chloride and ammonium sulfate, which is a combination of concentrations of each that improves the differentiation of mutant target polynucleotide from wild-type nucleic acid as determined by the Cq following an amplification reaction as compared to the Cq following an amplification reaction lacking the combination. For instance, an effective concentration of potassium chloride can be at least 20 mM to 80 mM, 30 mM to 80 mM, 40 mM to 70 mM, at least 40 mM to less than 70 mM, less than 70 mM, 60 mM, 40 mM to 48 mM, 45 mM, 10 mM to 40 mM. And, for instance, an effective concentration of ammonium sulfate can be at least 20 mM, 20 mM to 35 mM, at least 20 mM to less than 35 mM, less than 35 mM, 20 mM to 25 mM, 20 mM to 24 mM, 22 mM, 10 to 20 mM, or 15 mM. As shown in the Examples herein, in combination the particularly effective concentrations of potassium chloride and ammonium sulfate can be 60 mM and 15 mM, respectively; or 30 mM and 16 mM, respectively; or, preferably 45 mM and 22 mM, respectively. Other concentrations of each of potassium chloride and ammonium sulfate are also contemplated and can also be used, as may be determined by those of ordinary skill in the art (e.g., by determining whether the differentiation of target polynucleotide (e.g., a mutant or variant nucleic acid) from a more abundant target polynucleotide (e.g., a wild-type nucleic acid) in the methods described herein has been improved in the presence of particular concentrations as compared to other concentrations).

### Polymerases

The mixtures disclosed herein can also comprise at least one polymerase (e.g., a DNA polymerase) and at least one source of nucleotides (e.g., dNTPs). The polymerase can be a DNA polymerase with 5′ to 3′ exonuclease activity. In some embodiments, the polymerase can be a “thermostable polymerase,” which refers to an enzyme that is heat-stable, heat-resistant, and/or not irreversibly inactivated when subjected to elevated temperatures for the time necessary to effect destabilization of single-stranded nucleic acids or denaturation of double-stranded nucleic acids during amplification (e.g., will not irreversibly denature at about 90° to about 100° C. under conditions such as is typically required for amplification (e.g., in a polymerase chain reaction (PCR)) and catalyzes polymerization of deoxyribonucleotides to form primer extension products that are complementary to a target polynucleotide strand. Thermostable polymerases may be obtained, for example, from a variety of thermophilic bacteria that are commercially available (for example, from American Type Culture Collection, Rockville, Md.) using methods that are well-known to one of ordinary skill in the art (See, e.g., U.S. Pat. No. 6,245,533). Bacterial cells may be grown according to standard microbiological techniques, using culture media and incubation conditions suitable for growing active cultures of the particular species that are well-known to one of ordinary skill in the art (See, e.g., Brock, T. D., and Freeze, H., J. Bacteriol. 98(1):289-297 (1969); Oshima, T., and Imahori, K, Int. J. Syst. Bacteriol. 24(1):102-112 (1974)). Suitable for use as sources of thermostable polymerases are the thermophilic bacteria Thermus aquaticus, Thermus thermophilus, Thermococcus litoralis, Pyrococcus furiosus, Pyrococcus woosii, and other species of the Pyrococcus genus, Bacillus stearothermophilus, Sulfolobus acidocaldarius, Thermoplasma acidophilum, Thermus flavus, Thermus ruber, Thermus brockianus, Thermotoga neapolitana, Thermotoga maritima, and other species of the Thermotoga genus, and Methanobacterium thermoautotrophicum, and mutants of each of these species. Exemplary thermostable polymerases can include, but are not limited to, any of the SuperScript, Platinum, TaqMan, MicroAmp, AmpliTaq, and/or fusion polymerases. Exemplary polymerases can include but are not limited to Taq™ DNA polymerase, AmpliTaq DNA polymerase, AmpliTaq™ Gold DNA polymerase, DreamTaq™ DNA Polymerase, recombinant, modified form of the Thermus aquaticus DNA polymerase gene expressed in E. coli (Thermo Fisher Scientific), iTaq™ (Bio-Rad), Platinum Taq DNA Polymerase High Fidelity, Platinum™ II Taq™ Hot-Start DNA Polymerase, Platinum SuperFi DNA Polymerase, AccuPrime Taq™ DNA Polymerase High Fidelity, Tne DNA polymerase, Tma DNA polymerase, Phire Hot Start II DNA polymerase, Phusion U Hot Start DNA Polymerase, Phusion Hot Start II High-Fidelity DNA Polymerase, iProof High Fidelity DNA Polymerase (Bio-Rad); HotStart Taq Polymerase (Qiagen)), a chemically modified polymerase that for instance blocks its activity at a particular temperature such as room temperature), and/or mutants, derivatives and/or fragments thereof. In some embodiments, an oligonucleotide or aptamer may also be used as a hot start agent, and/or the hot start function may result from a chemical modification to a polymerase that blocks its activity at a particular temperature (e.g., room temperature) (e.g., TaqGold, FlashTaq, Hot-Start Taq). In some embodiments, the hot start component may be one or more antibodies directed to (i.e., have binding specificity for) a thermostable polymerase in the mixture (as available from Thermo Fisher Scientific in, e.g., Platinum™ II Hot-Start Green PCR Master Mix; DreamTaq™ Hot Start Green PCR Master Mix, Phusion U Green Muliplex PCR Master Mix, Phire Green Hot Start II Master Mix, or AmpliTaq® Gold 360 Master Mix (Thermo Fisher Scientific)). In some embodiments, a dual hot start mechanism may be used. For example, a first hot start component, such as an oligonucleotide may be used as a hot start agent in conjunction with a second hot start component, such as one or more antibodies. In some embodiments, the first and second hot start components of the dual hot start mechanism, may be the same type or different (oligo-based; antibody-based; chemical-based, etc.). In some embodiments, the first and second hot start components of the dual hot start mechanism may be inhibitory to the same polymerase (e.g., a dual hot start mechanism which employs an inhibitory antibody directed to Taq DNA polymerase and an inhibitory oligonucleotide specific to Taq DNA polymerase). In some embodiments, the polymerase can be a fusion or chimeric polymerase which refers to an enzyme or polymerase that is comprised of different domains or sequences derived from different sources. For example, a fusion polymerase may comprise a polymerase domain, such as a Thermus aquaticus (Taq) polymerase domain, fused with a DNA binding domain, such as a single- or double-stranded DNA binding protein domain. Fusion or chimeric polymerases may be obtained, for example, using methods that are well-known to one of ordinary skill in the art (See, e.g., U.S. Pat. No. 8,828,700), the disclosure of which is incorporated by reference in its entirety. In some embodiments, such fusion or chimeric polymerases are thermostable. In some embodiments, the mixtures can comprise a mixture that is a master mix and/or a reaction mixture (e.g., TaqPath™ ProAmp™ Master Mix (Applied Biosystems™), TaqPath™ ProAmp™ Multiplex Master Mix (Applied Biosystems™), TaqMan™ PreAmp Master Mix (Applied Biosystems™) TaqMan™ Universal Master Mix II with UNG (Applied Biosystems™), TaqMan™ Universal PCR Master Mix II (no UNG) (Applied Biosystems™), TaqMan™ Gene Expression Master Mix II with UNG (Applied Biosystems™), EXPRESS qPCR Supermix, universal (Invitrogen), TaqMan™ Fast Advanced Master Mix (Applied Biosystems™), TaqMan™ Multiplex Master Mix (Applied Biosystems™), TaqMan™ PreAmp Master Mix Kit (Applied Biosystems™), TaqMan™ Universal PCR Master Mix, no AmpErase™ UNG (Applied Biosystems™), PowerUp SYBR Green Master Mix (Applied Biosystems™), or FlashTaq HotStart 2X MeanGreen Master Mix (Empirical Biosciences)). In some embodiments, the mixtures can further comprise one or more of at least one detergent; glycerol; and at least one reference dye (e.g., ROX™ Mustang Purple™). In some embodiments, the reaction mixture further can comprise an amplicon(s) comprising the target polynucleotide sequence (e.g., first sequence) of the target polynucleotide strand. In some embodiments, the mixture does not include an amplicon that includes a sequence of a second polynucleotide strand (e.g., of a major allelic variant).

### Target Polynucleotide

In some embodiments, the mixtures disclosed herein include a nucleic acid sample suspected of comprising the target polynucleotide strand (e.g., a target polynucleotide sequence or target polynucleotide). The target polynucleotide sequence (e.g., target polynucleotide) may be any suitable single-, double-, or otherwise configured polynucleotide to which the target sequence-specific primer (e.g., the first oligonucleotide, TSP), the locus-specific primer (e.g., the second oligonucleotide, LSP), and the target site-specific probe (e.g., the third oligonucleotide) can bind and support amplification thereof. In some embodiments, the nucleic acid sample can be deoxyribonucleic acid (DNA), such as genomic DNA (gDNA) or complementary DNA (cDNA). Thus, in some embodiments, the mixtures disclosed herein may include a single-stranded target polynucleotide including the target polynucleotide strand; and/or, a double-stranded target polynucleotide including the target polynucleotide strand and a target complement polynucleotide strand, wherein the target complement polynucleotide strand is substantially complementary to the target polynucleotide strand. In some embodiments, the double-stranded target polynucleotide including the target polynucleotide strand and a target complement polynucleotide strand, wherein the target complement polynucleotide strand is substantially complementary to the target polynucleotide strand, and a double-stranded target polynucleotide including a variant polynucleotide strand and a variant complement polynucleotide strand, wherein the variant polynucleotide strand has identity to or is substantially identical to the target polynucleotide strand and comprises a different nucleotide at the target variant nucleotide than the target polynucleotide strand, and wherein the variant complement polynucleotide strand is substantially complementary to the variant polynucleotide strand.

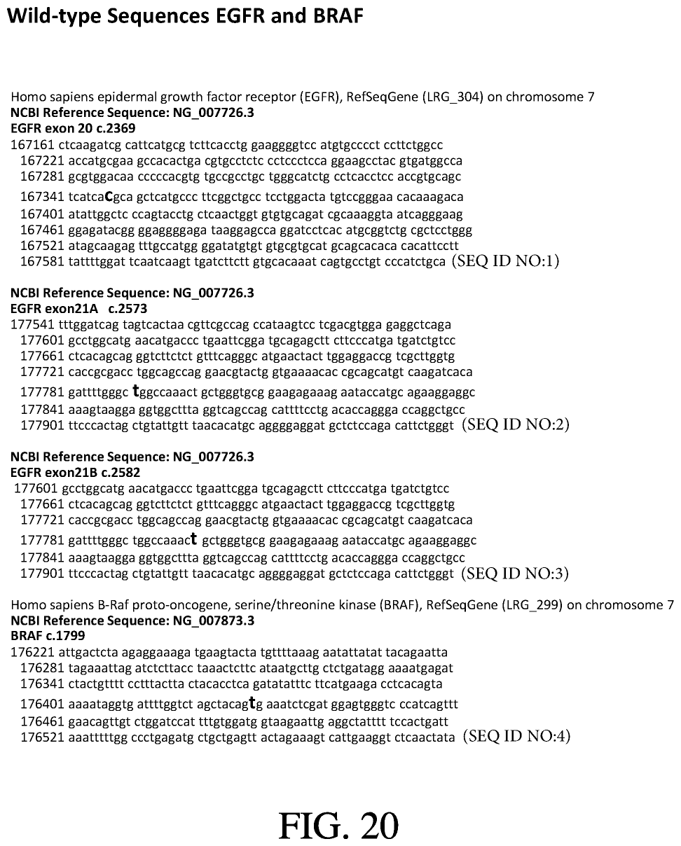

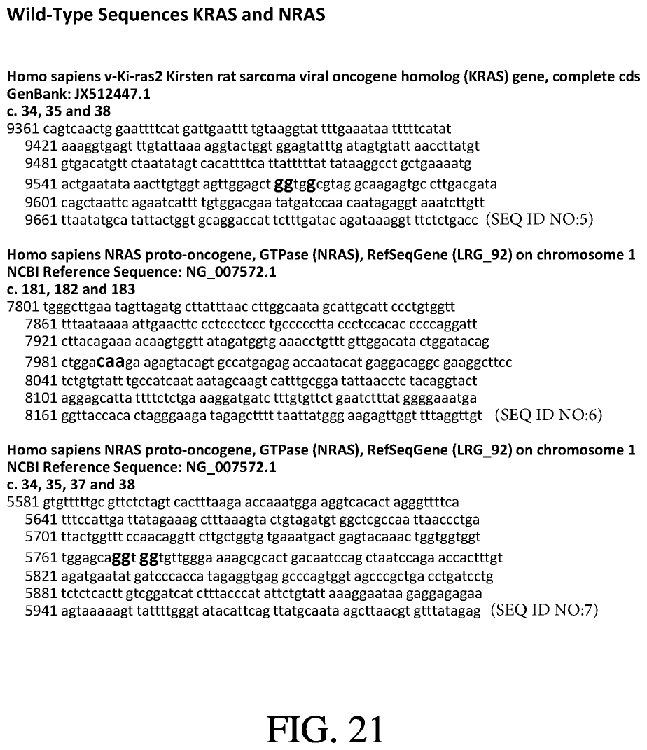

As mentioned above, the target polynucleotide sequence can include the target variant nucleotide which can “correspond to”, be hybridizable to, be associated with, and/or be found within an allele (i.e., an allelic variant such as may be represented by a SNP and/or mutation). Such SNPs or mutations may include, but are not limited to those found in, for example, EGFR (epidermal growth factor receptor) (e.g., FIG. 20), a KRAS (e.g., in codon 12 and/or codon 13; or an NRAS mutation (e.g., FIG. 21), Kit, pTEN TP53, ESR1, PIK2CA, TSC1, MDM2, ERBB2, SMAD4, and FGFR2 genes, including those mutations listed in Table 1 and Table 2. For instance, as shown in the examples herein, the mixtures and methods of this disclosure can be used to identify and/or quantitate KRAS with any of the following exemplary, non-limiting mutations: a guanosine-to-adenosine (G>A (GGT>GAT)) mutation, encoding a glycine-to-aspartate (G12D) substitution at amino acid 12 of the translated protein; a guanosine-to-thymidine (G>T (GGT>GTT)) mutation encoding a glycine-to-valine (G12V) substitution at amino acid 12 of the translated protein; a guanosine-to-thymidine (G>T (GGT>TGT)) mutation encoding a glycine-to-cysteine (G12C) substitution at amino acid 12 of the translated protein; guanosine-to-adenosine (G>A (GGT>AGT)) mutation encoding a glycine-to-serine (G125) substitution at amino acid 12 of the translated protein; guanosine-to-cytosine (G>C (GGT>GCT)) mutation encoding a glycine-to-alanine (G12A) substitution at amino acid 12 of the translated protein; a guanosine-to-cytosine (G>C (GGT>CGT)) mutation encoding a glycine-to-arginine (G12R) substitution at amino acid 12 of the translated protein; and/or, a guanosine-to-adenosine (G>A (GGC>GAC)) mutation encoding a glycine-to-aspartate (G13D) substitution at amino acid 13 of the translated protein. Other KRAS mutations, as described above, as well as other KRAS mutations beyond those listed in Table 2 are also contemplated and may also be suitable for analysis and/or detection using the reagent and methods described herein as would be understood by those of ordinary skill in the art.

As also shown in the examples herein, the compositions and/or mixtures and methods of this disclosure can be used to identify and/or quantitate NRAS with any of the following exemplary, non-limiting mutations: a guanine-to-adenosine (G>A) mutation encoding a glycine-to-aspartic acid mutation at amino acid 12 (G12D) of the translated protein; a guanosine to adenosine (G>A) mutation encoding a glycine-to-aspartic acid mutation at amino acid 13 (G13D) of the translated protein; an adenosine-to-thymidine (A>T) mutation a glutamine-to-lysine mutation at amino acid 61 of the translated protein; an adenosine-to-guanosine (A>G) mutation encoding a glutamine-to-arginine mutation at amino acid 61 (Q61R) of the translated protein; an adenosine-to-thymidine (A>T) mutation, encoding a glutamine-to-histidine mutation at amino acid 61 (Q61H) of the translated protein; and/or, an adenosine-to-thymidine (A>T) mutation encoding a glutamine-to-leucine mutation at amino acid 61 (Q61L) of the translated protein. Other NRAS mutations, as described above, as well as other NRAS mutations beyond those listed in Table 2 are also contemplated and may also be suitable for analysis and/or detection using the reagent and methods described herein as would be understood by those of ordinary skill in the art.

As also shown in the examples herein, the compositions and/or mixtures and methods of this disclosure can be used to identify and/or quantitate EGFR with any of the following exemplary, non-limiting mutations: a cytosine-to-thymidine (C>T) mutation encoding a threonine-to-methionine mutation at amino acid 790 of the translated protein (EGFR20); a deletion of nucleotides 746-750 of the wild-type coding sequence (EGFR19); and/or, a thymidine-to-adenosine (T>A) mutation encoding a leucine-to-glutamic acid mutation at amino acid 861 (L861Q) of the translated protein. Other EGFR mutations, as described above, as well as other EGFR mutations beyond those listed in Table 2 are also contemplated and may also be suitable for analysis and/or detection using the reagent and methods described herein as would be understood by those of ordinary skill in the art.

As also shown in the examples herein, the compositions and/or mixtures and methods of this disclosure can be used to identify and/or quantitate BRAF with, as a non-limiting example, a thymidine-to-adenosine mutation encoding a valine-to-glutamic acid mutation at amino acid 600 (V600E) of the translated protein. Other BRAF mutations, as described above, as well as other BRAF mutations beyond those listed in Table 2 are also contemplated and may also be suitable for analysis and/or detection using the reagent and methods described herein as would be understood by those of ordinary skill in the art.

As also shown in the examples herein, the compositions and/or mixtures and methods of this disclosure can be used to identify and/or quantitate: ESR1 with any of the following exemplary, non-limiting mutations: a guanine-to-cytosine (G>C) mutation encoding a glutamic acid-to-glutamine mutation at amino acid 380 (E380Q) of the translated protein; PIK3CA with any of the following exemplary, non-limiting mutations: an adenosine-to-guanine (A>G) mutation encoding a histidine-to-arginine mutation at amino acid 1047 (H1047R) of the translated protein; TP53 with any of the following exemplary, non-limiting mutations: a guanine-to-adenosine (G>A) mutation encoding an arginine-to-histidine mutation at amino acid 273 (R273H) of the translated protein; a thymidine-to-adenosine (T>A) mutation encoding a histidine-to-glutamine mutation at amino acid 179 (H179Q) of the translated protein; a adenosine-to guanine (A>G) mutation encoding a tyrosine-to-cysteine mutation at amino acid 220 (Y220C) of the translated protein; a guanine-to-thymidine (G>T) mutation encoding an arginine-to-methionine mutation at amino acid 179 (R249M) of the translated protein. Other ESR1, PIK3CA, and TP53 mutations, as described above, as well as other ESR1, PIK3CA, and TP53 mutations beyond those listed in Table 1 and/or Table 2 are also contemplated and may also be suitable for analysis and/or detection using the reagent and methods described herein as would be understood by those of ordinary skill in the art.

Samples in which target polynucleotide may exist include, for instance, tissue, cell, and/or fluid (e.g., circulating, dried, reconstituted) of a mammalian or non-mammalian organism (e.g., including but not limited to a plant, virus, bacteriophage, bacteria, fungus, and/or other organism). In some embodiments, the sample may be, or be derived from, for example, mammalian saliva, buccal epithelial cell, cheek tissue, lymph, cerebrospinal fluid, skin, hair, blood, plasma, urine, feces, semen, tumor sample (e.g., a cancer cell), cultured cell, cultured tumor cell. The target polynucleotide may be DNA in genomic form, or it may be cloned in plasmids, bacteriophage, bacterial artificial chromosomes (BACs), yeast artificial chromosomes (YACs), and/or other vectors. Other types of samples may also be useful in the methods described herein which may be related, for example, to diagnostic or forensic assays.

### Lyophilization

In some embodiments, individual types of oligonucleotides and/or mixtures of the same can comprise additional components appropriate for lyophilization and/or be lyophilized and/or otherwise stabilized (e.g., freeze-dried (e.g., freezing, primary drying, secondary drying) or prepared as an evaporated composition) and, therefore, can include components or be processed to provide for such stabilization. The mixtures can be prepared as compositions that are stable for approximately two years at −20° C. (e.g., dry, or in a solution of water or TE buffer (10 mM Tris, pH 7.5 to 8, 1 mM EDTA); approximately one year at 4° C. (e.g., dry, or in a solution of water or TE buffer); approximately three to six months at room temperature (e.g., dry, or in a solution of water or TE buffer); and/or approximately one to two months at a temperature higher than room temperature (e.g., dry, or in a solution of water or TE buffer). Kits, described below, may also include a buffer or the like for reconstitution of lyophilized or otherwise stabilized the oligonucleotides and/or mixtures (e.g., water (e.g., sterile, nuclease-free water) or a weak buffer such as TE or Tris (10 mM Tris-HCl, pH 8.0)).

### Methods

Polymerase chain reaction (PCR) refers generally to cycling polymerase-mediated exponential amplification of nucleic acids employing primers that hybridize to complementary strands of a target polynucleotide that are typically carried out using thermostable enzymes and/or thermocycler devices designed to perform such reactions, as described for example in Innis et al., PCR Protocols: A Guide to Methods and Applications, Academic Press (1990). Suitable PCRs may include but are not limited to real-time PCR (e.g., quantitative PCR (qPCR)), nested PCR, multiplex PCR, end point PCR, digital PCR (dPCR), drop dPCR, isothermal PCR, touchdown PCR, co-amplification at lower denaturation temperature (COLD) PCR, and/or isothermal PCR. In preferred embodiments, the PCR may be real-time PCR (e.g., quantitative PCR (qPCR)). Those of ordinary skill in the art understand that the melting temperature (“Tₘ”) of an oligonucleotide can significantly affect PCR performance. The Tₘ of an oligonucleotide refers to the temperature (typically in degrees Celsius) at which 50% of the polynucleotides in a population of a single-stranded oligonucleotide are hybridized to their complementary sequence and 50% of the polynucleotides in the population are not-hybridized to said complementary sequence. As is understood by those of ordinary skill in the art, the Tₘ of an oligonucleotide (e.g., a primer) can be determined empirically by means of a melting curve. The Tm can depend on the primer length, percentage of GC content, molecular weight, and extinction coefficient thereof. In some cases, the Tₘ can be calculated using formulas and/or calculators well-known in the art (See, e.g., Maniatis, T., et al., Molecular cloning: a laboratory manual/Cold Spring Harbor Laboratory, Cold Spring Harbor, N.Y.: 1982; Thermo-Fisher's Tₘ Calculator available at Thermo Fisher.com; TaqPipe; PrimerExpress). Those of ordinary skill in the art also understand that the Tₘ of the primers used in PCR (e.g., a primer pair) can be used to determine the appropriate annealing temperature, and that the specificity and yield of PCR can also depend on primer concentration as well as the polymerase used. Particular calculations may need to be made based on which polymerase is used. For instance, the modified Allawi & SantaLucia's thermodynamics method can be used for Tₘ and annealing temperature calculation of reactions with Platinum SuperFi, Phusion and Phire DNA Polymerases (Biochemistry, 36(34): 10581-94 (1997)). In some embodiments, the annealing temperature of PCR may be slightly higher (e.g., within 5-10 degrees) than the lowest Tₘ of an oligonucleotide of particular primer pair, and higher than the Tₘ of both oligonucleotides. In some embodiments, the Tₘ of the first oligonucleotide is within 5° C. of the Tₘ of the second oligonucleotide. In some embodiments, the Tₘ of the first oligonucleotide can be 48 to 58° C. and the Tₘ of the second oligonucleotide can be 48 to 58° C. In some embodiments, the Tₘ of the third oligonucleotide can be at least 5° C. and no more than 25° C. higher than the Tₘ of the first oligonucleotide. In some embodiments, the Tₘ of the third oligonucleotide can be at least 8° C. and no more than 12° C. higher than the Tₘ of the first oligonucleotide. In some embodiments, the annealing temperature of PCR using the primers disclosed herein (e.g., the first and second oligonucleotides) can be within 5-10° C. of the higher Tₘ of a particular primer pair. For instance, where the Tₘ of the first oligonucleotide is within 5° C. of the Tₘ of the second oligonucleotide, the annealing temperature could be within 5° C. of the higher calculated Tₘ of said oligonucleotides. In some embodiments, such as where the Tₘ of the first oligonucleotide can be 48 to 58° C. and the Tₘ of the second oligonucleotide can be 48 to 58° C., for example, the annealing temperature could be above 48° C. or 58° C. (e.g., within about 5° C. of the higher Tₘ). Other conditions for PCR are also contemplated by this disclosure as would be understood by those of ordinary skill in the art.

In some embodiments, a PCR reaction can include an “enrichment phase” or “enrichment cycle(s)” in which low abundance nucleic acids (i.e., target polynucleotides or target polynucleotide molecules) were amplified in preference to more abundant nucleic acids (e.g., major alleles, wild-type nucleic acids), the mixtures being subjected to 1 cycle of 95° C. for 2 min; 15-20 cycles of 95° C. for 1-3 sec and 64° C. for 20 sec (enrichment phase); and 40 cycles of 95° C. for 1 sec and 60° C. for 20 sec (amplification and detection phase). Not to be limited by theory, it is possible that the elevated temperature in the enrichment phase favors annealing of the target sequence-specific primers to the low abundance target polynucleotides, which form a full match when bound to the target sequence-specific primer, as compared to annealing of the target sequence-specific primers to the abundant nucleic acids, which contain a single base mismatch (e.g., a single base mismatch) when bound to the target sequence-specific primer. In some embodiments, qPCR reactions were performed without an enrichment phase, and the mixtures subjected to: 1 cycle of 95° C. for 1-10 min, for example 2 min; and 40 cycles of 95° C. for 1 sec and 60° C. for 20 sec (amplification and detection phase). In some embodiments, enrichment can be carried out using PCR comprising from 15-25 cycles at a temperature of 12-16° C. higher than the calculated melting temperature (Tₘ) of the first oligonucleotide (e.g., the TSP) and/or the second oligonucleotide (e.g., the LSP). The accepted guideline for anneal/extend temperature is 5° C. lower than the calculated primer Tₘ. The approach described here uses elevated temperature in the enrichment phase to favor annealing and amplification of the target polynucleotide (i.e., less abundant or rare nucleic acid molecule) at the expense of the abundant target polynucleotide sequence (e.g., major allele, wild-type nucleic acid). The TSP is a perfect match for the (rare) target polynucleotide sequence, but includes a single base mismatch with the more abundant target polynucleotide (e.g., major allele, wild-type nucleic acid). This mismatch combined with higher anneal/extension temperatures enables amplification of the less abundant polynucleotide species (i.e., the less abundant and/or rare target polynucleotide molecule). The anneal/extend temperature is typically 3° C. to 8° C. lower than the enrichment temperature. If the enrichment and anneal/extend temperatures are equivalent, discrimination of the rare target from the abundant target will be lost. In some embodiments, qPCR may be carried out in serial dilutions (e.g., 5-log dilutions) using a minimum of three replicates, especially for samples anticipated to include low copy numbers of the target polynucleotide in a sample (with statistical analysis of the results). Other conditions for PCR are also contemplated by this disclosure as would be understood by those of ordinary skill in the art.

In some embodiments, this disclosure provides methods for detecting a target polynucleotide including a target variant nucleotide by forming a reaction mixture of a test nucleic acid sample potentially comprising a target polynucleotide sequence; and a mixture of a first oligonucleotide (e.g., TSP), second oligonucleotide (e.g., LSP), and third oligonucleotide (e.g., target site-specific probe; carrying out an amplification reaction to produce amplicons using at least the first and second oligonucleotides as primers; and, detecting the amplicons by detecting a change in a detectable property of the third oligonucleotide (e.g., target site-specific probe; wherein the detection of amplicons indicates the target polynucleotide is present within the test nucleic acid sample. In some embodiments, the test nucleic acid sample comprises a mixture of nucleic acids comprising target polynucleotides that includes or corresponds to the target variant nucleotide, and wild-type nucleic acids that do not include or correspond to the target variant nucleotide. In some embodiments, as described above and shown in the examples, the method may include enriching (e.g., increasing) the number of target polynucleotides in the test nucleic acid sample relative to more abundant (e.g., wild-type) nucleic acid polynucleotides by, for instance, carrying out an amplification reaction under conditions different than those used to amplify the target polynucleotide sequence for detection (i.e., the enrichment phase comprising, e.g., carrying out PCR comprising from 15-25 cycles at a temperature of 12-16° C. higher than the calculated melting temperature (Tₘ) of the first oligonucleotide; and then carrying out 15 to 60, 20-50, 30-50, 35-45, 38-42, or 40 cycles at about 5° C. to 25° C. of the Tₘ of the third oligonucleotide and three to eight degrees Celsius below the temperature at which enrichment was carried out).

Amplification resulting from PCR is typically quantified by measuring the threshold cycle (C₁), a relative measure of the concentration of amplified nucleic acids in a sample, which is the intersection between an amplification curve and a threshold line. Cₜ data is displayed on an amplification plot showing the variation of log (ΔRn) with the PCR cycle number. Rn is the fluorescence of a reporter dye divided by the fluorescence of a passive reference dye, i.e., Rn is the reporter signal normalized to the fluorescence signal of the reporter dye. Rn is plotted against the PCR cycle number. ΔRn is Rn less the baseline fluorescence value (e.g., background FAM fluorescence) which may vary depending on the amplification conditions (e.g., the type of reporter dye used and/or the type of master mix utilized). Exemplary reporter dyes (or passive reference dyes) include but are not limited to ROX™ or Mustang Purple™. The Cₜ value increases with a decreasing amount of template. In some embodiments, such as those shown in the examples herein, multiple qPCR reactions (e.g., two or more) may be carried out in the same mixture (e.g., control and test reactions), leading to two Cₜ values that are differentiated from one another on the amplification plot and may be expressed as a delta Cₜ (ΔCₜ). In some embodiments, the ΔCₜ may be, for instance, at least about 8 (e.g., Example 1 herein exhibits ΔCₜ values of from 9.0 to 16.3). The data generated from the amplification reactions can be exported in Excel format, the Ct (alternatively referred to as Cq) values for replicate reactions averaged, and the delta average Cq of labeled target polynucleotides (e.g., FAM and VIC targets) in each condition determined and plotted. Thus, the delta Cq can be used to quantify in a relative or absolute manner, a starting amount of target polynucleotide or target variant polynucleotide (e.g., as shown in the examples and figures of this disclosure).

In some embodiments, the compositions and/or mixtures disclosed here may comprise ribonucleic acid (RNA) that may serve as the starting material in, for instance, reverse transcriptase PCR (RT-PCR) using the compositions and/or mixtures, and methods disclosed herein. In such embodiments, the compositions and/or mixtures may comprise a reverse transcriptase (RT) and related components. In some embodiments, the RT-PCR may be a one-step procedure using one or more target sequence-specific primers (e.g., TSP or first oligonucleotide), one or more locus-specific primers (e.g., LSP or second oligonucleotide), and one or more target site-specific probes (e.g., third oligonucleotide). Suitable exemplary RTs can include, for instance, SuperScript Reverse Transcriptases (Thermo Fisher Scientific), SuperScript IV Reverse Transcriptases (Thermo Fisher Scientific), or Maxima Reverse Transcriptases (Thermo Fisher Scientific). The compositions and/or mixtures may also comprise any other components necessary for carrying out such reactions, such as may be found in SuperScript IV VILO Master Mix (Thermo Fisher Scientific), or any other suitable master mixes (including those described above).

Devices have been developed that can perform thermal cycling reactions with compositions containing fluorescent indicators which are able to emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; 6,174,670; and 6,814,934 and include, but are not limited to, the Prism® 7700 Sequence Detection System (Thermo Fisher Scientific), the ABI GeneAmp® 5700 Sequence Detection System (Thermo Fisher Scientific), the ABI GeneAmp® 7300 Sequence Detection System (Thermo Fisher Scientific), the ABI GeneAmp® 7500 Sequence Detection System (Thermo Fisher Scientific), the StepOne™ Real-Time PCR System (Thermo Fisher Scientific), the ABI GeneAmp® 7900 Sequence Detection System (Thermo Fisher Scientific), QuantStudio 12K Flex Real-Time PCR System (Thermo Fisher Scientific), QuantStudio 7 Flex Real-Time PCR System (Thermo Fisher Scientific), QuantStudio 6 Flex Real-Time PCR System (Thermo Fisher Scientific), QuantStudio 5 Flex Real-Time PCR System (Thermo Fisher Scientific), QuantStudio 3 Flex Real-Time PCR System (Thermo Fisher Scientific), ViiA Real-Time PCR System (Thermo Fisher Scientific), C1000 Touch™ Thermal Cycler (Bio-Rad), S1000™ Thermal Cycler (Bio-Rad), T100™ Thermal Cycler (Bio-Rad), CFX96 Touch™ (Bio-Rad), CFX384 Touch™ (Bio-Rad), CFX Connect™ (Bio-Rad), and Rotor-Gene Q (Qiagen), among others; and systems thereto (e.g., software). As is well-known to those of skill in the art, these systems can be used to simultaneously analyze multiple samples (e.g., 96-well or 384-well systems) and/or multiple detectable labels (e.g., in multiplex assays) and the like, and are suitable for use with the mixtures and methods described herein. For instance, these devices can include multiple channels for detecting the different detectable labels (e.g., two channels for detecting green and yellow; four channels for detecting green, yellow, orange, and red; five channels for detecting green, yellow, orange, red and crimson; six channels for detecting blue, green, yellow, orange, red and crimson, and so on). It is also well-known that many of these systems may be automated and/or rely on and/or use software (e.g., Applied Biosystems QuantStudio™ (Thermo Fisher Scientific), CFX Maestro Software (Bio-Rad), CFX Automation System II (Bio-Rad), Q-Rex software (Qiagen)). Any of the available PCR formats may also be utilized, including but not limited to tubes (e.g., 0.1 or 0.2 ml), cards, plates (e.g., microplates; 48-, 60-, 96- or 384-well plates), arrays, open arrays, microfluidics, and/or any plastic or other parts designed for use with particular devices used in PCR. Any of these devices and software, and/or any others available to those of ordinary skill in the art, may also be suitable and are contemplated herein.

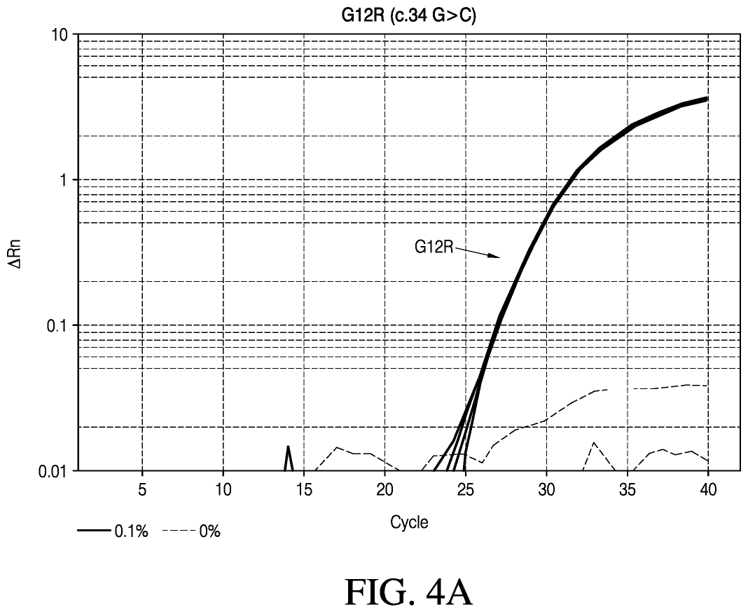

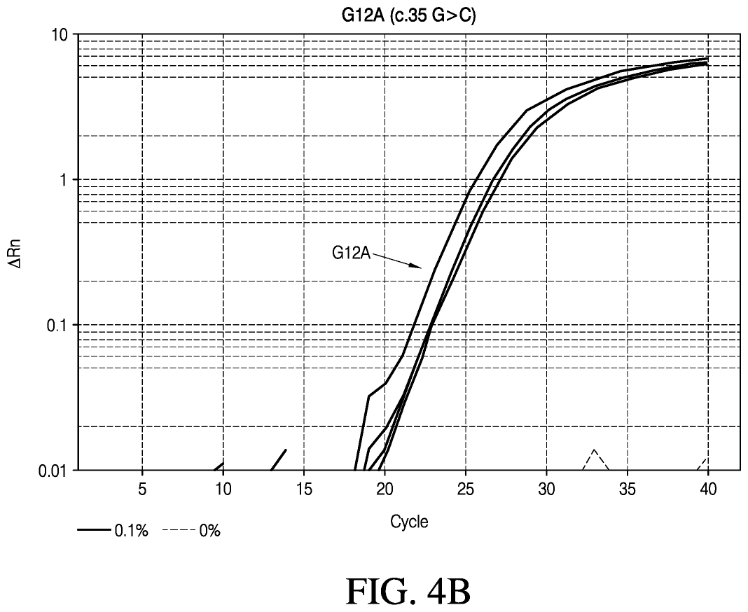

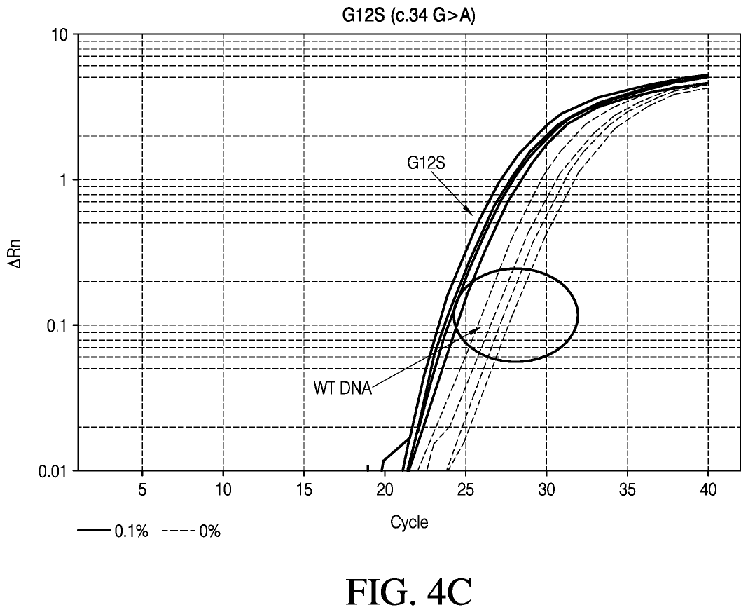

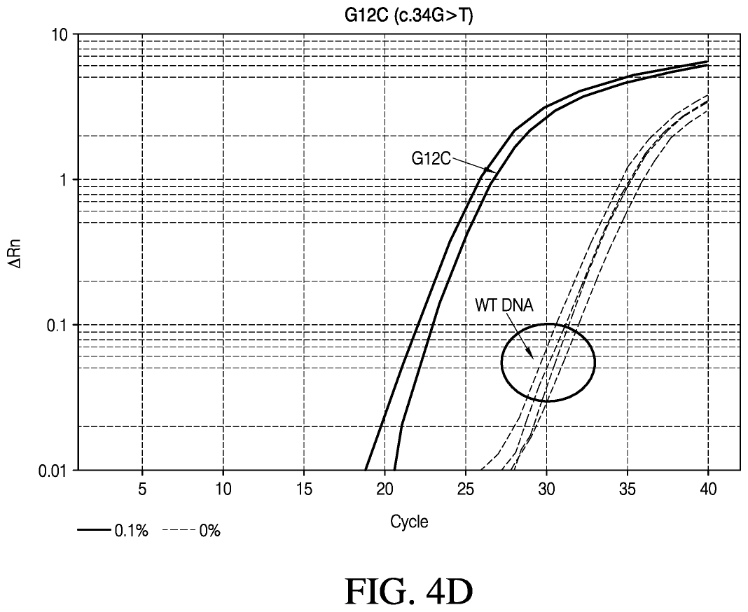

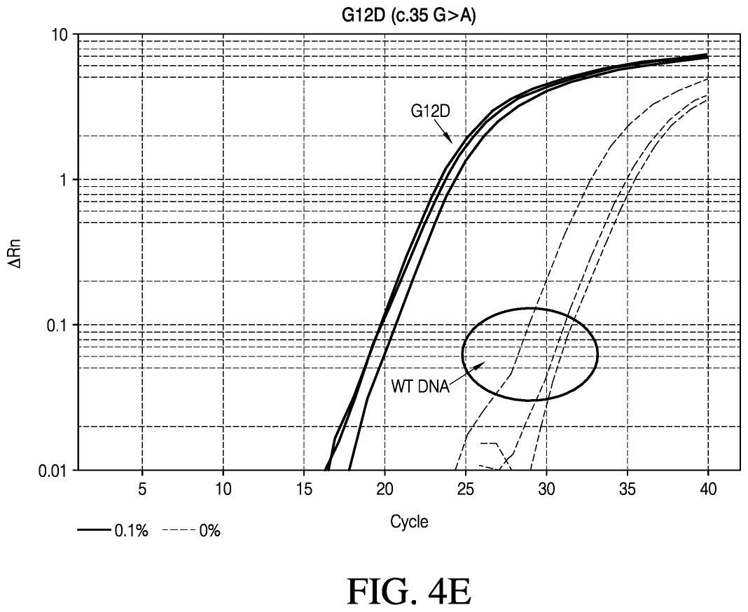

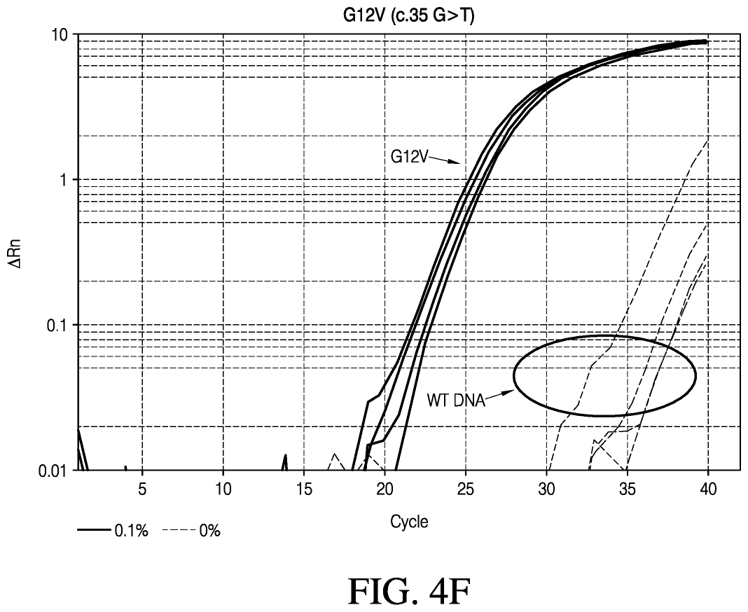

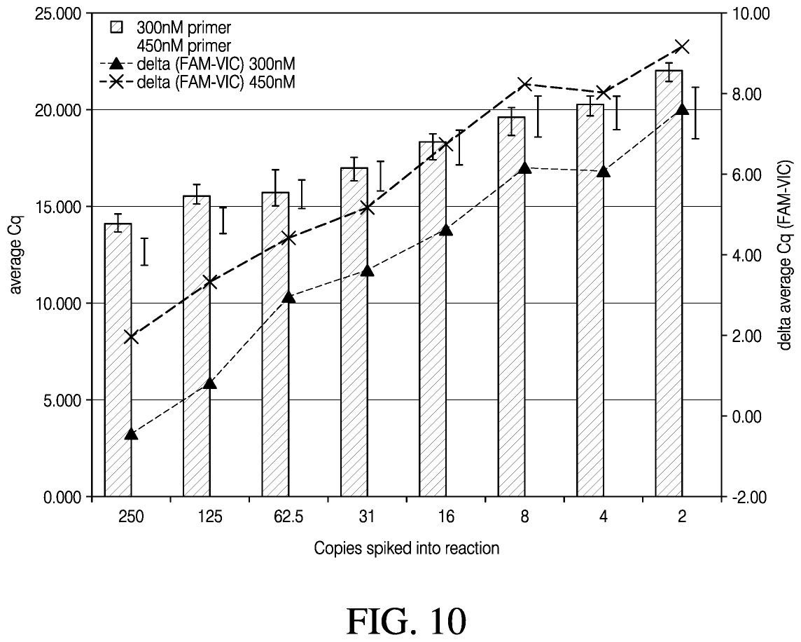

In some embodiments, the methods disclosed here can detect a target variant nucleotide indicative of a mutation, wherein the nucleic acid sample comprises about any of as few as one; two; three; four; five; six; seven; eight; nine; 10; 1 to about 10; about 10-15; about 15-20; about 20-25; about 26-50; about 50-75; or about 75-100 copies of the target polynucleotide (e.g., in the presence of a much larger number of more abundant polynucleotides). For example, the major allele(s) and/or wild-type polynucleotides may comprise more than 99% of the polynucleotides and/or the target polynucleotides comprise about any of, for example, 2%, 1%, 0.1%, 0.01%, 0.001% or 0.0001% of a sample polynucleotide population (e.g., a test sample). The target polynucleotide can detected from within such a sample polynucleotide population using the methods described herein. In some embodiments, methods for detecting and/or quantitating a low abundance (e.g., rare) allelic variant comprising a target variant nucleotide in a pooled or mixed sample comprising other alleles. In some embodiments, the target variant nucleotide includes a purine base and a wild-type nucleotide at the target variant nucleotide position includes a different purine base; the target variant nucleotide includes a pyrimidine base and a wild-type nucleotide at the target variant nucleotide position includes a different pyrimidine base. In some embodiments, the target site-specific probe (e.g., the third oligonucleotide) has a Tₘ 6-20° C. (optimally 8-12° C.) above the Tₘ of the TSP (e.g., the first oligonucleotide) and the amplification reaction (e.g., qPCR) is carried out with an annealing temperature within 5° C. of the Tₘ of the TSP. In some embodiments, the test nucleic acid sample is derived from a mammalian or non-mammalian animal tissue or cell, or a plant tissue or cell. In some embodiments, detection of amplicons indicates the presence of cancer cells within a tissue from which the test nucleic acid sample was derived. In some embodiments, the target polynucleotide includes at least one mutation in Ras, EFGR, Kit, pTEN, and/or p53; and/or at least one KRAS or NRAS mutation. In some embodiments, the amplification reactions carried out in these methods are or rely upon or include the polymerase chain reaction (PCR) including but not limited to real-time PCR. For instance, in certain examples below, the target variant nucleotide present within allelic variant KRAS DNA (which may be referred to elsewhere herein as target polynucleotides); the forward or reverse primer (the TSP) was designed to bind the target variant nucleotide by including a nucleotide at the 3′ end complementary to the target variant nucleotide of the allelic variant KRAS DNA being assayed; the labeled TaqMan probe was designed to include the target variant nucleotide; and the target polynucleotide was amplified and detected (see, e.g., FIG. 4D differentiating the Cₜ values of KRAS G12C and wild-type (WT) DNA, or FIG. 10 showing variances in Cₜ values based on the amount of target polynucleotide in a sample). Other embodiments are also contemplated as will be understood by those of ordinary skill in the art.

In certain embodiments, the methods disclosed herein can be used to detect a target polynucleotide sequence (e.g., a first allelic variant) that is present in a sample at a frequency less than 1/10, 1/100, 1/1,000, 1/10,000, 1/100,000, 1/1,000,000, 1/10,000,000, 1/100,000,000 or 1/1,000,000,000, and any fractional ranges in between, of a wild-type nucleic acid sequence (e.g., second allelic variant) for a given nucleic acid sequence (e.g., SNP or gene). In some embodiments, the methods disclosed herein can be used to detect a target polynucleotide sequence (e.g., a first allelic variant) that is present in less than 2, 3, 4, 5, 6, 7, 8, 9, 10, 15, 20, 25, 50, 75, 100, 250, 500, 750, 1,000, 2,500, 5,000, 7,500, 10,000, 25,000, 50,000, 75,000, 100,000, 250,000, 500,000, 750,000, 1,000,000 copies per 1, 10, 100, 1,000 micro liters, and any fractional ranges in between, of a sample (e.g., test sample) or a reaction volume. In some embodiments the target polynucleotide sequence (e.g., a first allelic variant) can be a mutant nucleic acid sequence. In some embodiments the second allelic variant is wild-type nucleic acid sequence. In some embodiments, the present methods can involve detecting one target polynucleotide sequence (e.g., a first allelic variant, mutant) in a background of at least 1,000 to 1,000,000, such as about 1000 to 10,000, about 10,000 to 100,000, or about 100,000 to 1,000,000 wild-type polynucleotides, or any fractional ranges in between. In some embodiments, the methods can provide high sensitivity and the efficiency at least comparable to that of TaqMan®-based assays. In some embodiments, a comparison of the first amplicons (e.g., representing the target polynucleotide sequence) and the second amplicons (e.g., representing the wild-type nucleic acid sequence) involving the methods disclosed herein can provide improvements in specificity from 100 times to 1,000,000 times fold difference, such as about 100 to 1,000 times, about 1,000 to 10,000 times, about 10,000 to 100,000 times, or about 100,000 to 1,000,000 times fold difference, or any fractional ranges in between. In some embodiments, the size of the amplicons range from about 60-120 nucleotides long.

In some embodiments, the oligonucleotides, mixtures, compositions, methods, and/or kits comprising or relating to the same (e.g., as described herein) can be used for genotyping tetra-, tri- and di-allelic SNPs. In some embodiments, the compositions, methods, and/or kits can be used for DNA typing from mixed DNA samples for quality control (QC) and human identification assays, cell line QC for cell contaminations, allelic gene expression analysis, virus typing/rare pathogen detection, mutation detection from pooled samples, detection of circulating tumor cells in blood, and/or prenatal diagnostics. In some embodiments, the oligonucleotides, mixtures, compositions, methods, and/or kits comprising or relating to the same (e.g., as described herein) can be used to detect tumor cells in blood for early cancer diagnosis. In some embodiments, the compositions, methods, and/or kits can be used for cancer or disease-associated genetic variation or somatic mutation detection and validation. In some embodiments, the oligonucleotides, mixtures, compositions, methods, and/or kits comprising or relating to the same (e.g., as described herein) can be compatible with various instruments such as, for example, SDS software-based instruments from Applied Biosystems (Foster City, Calif.). Other uses and applications of the oligonucleotides, mixtures, compositions, methods, and/or kits comprising or relating to the same (e.g., as described herein) disclosed here are also contemplated by this disclosure as would be understood by those of ordinary skill in the art.

### Kits

This disclosure also provides kits for using the oligonucleotides, mixtures, and compositions, disclosed herein and/or carrying out the methods described herein. In some embodiments, this disclosure provides kits for quantitating a target polynucleotide sequence (e.g., a first allelic variant, mutant) in a sample comprising high abundance nucleic acid sequences (e.g., second allelic variants; wild-type nucleic acid sequences) that include: (a) a target sequence-specific oligonucleotide capable of hybridizing to a target variant nucleotide in a target polynucleotide (e.g., first oligonucleotide or first allele-specific primer); (b) a locus-specific oligonucleotide (e.g., second oligonucleotide, or second allele-specific primer); and, (c) a probe (e.g., a third oligonucleotide) capable of hybridizing to a nucleotide corresponding to a target variant nucleotide of a target polynucleotide sequence that is typically detectable (e.g., comprises a detectable label), and includes a quenching moiety and/or a MGB moiety. Optionally, a polymerase and/or other reaction components, such as dNTPs and/or cofactors such as Mg²⁺, may also be included in a kit. In some embodiments, the kit may comprise reagents and the like required to carry out RT-PCR (e.g., as described herein), where the one or more RTs may be contained in the same or separate containers from the DNA polymerase(s) that may be included in the kit. In some embodiments, the kit can include two or more containers comprising such components independently distributed in such containers, or included together in any combination in one or another containers. In some embodiments, such kits can include a first container containing a mixture provided herein (or any component thereof) and a second container containing a control nucleic acid sample including the first target polynucleotide strand. Thus, in some embodiments, the kit comprises a first container containing (a) a target sequence-specific oligonucleotide capable of hybridizing to a target variant nucleotide in a target polynucleotide (e.g., first oligonucleotide or first allele-specific primer); (b) a locus-specific oligonucleotide (e.g., second oligonucleotide, or second allele-specific primer); and, (c) a probe capable of hybridizing to a nucleotide corresponding to a target variant nucleotide of a target polynucleotide sequence (e.g., a third oligonucleotide) that is typically detectable (e.g., comprises a detectable label), and includes a quenching moiety and/or a MGB moiety (e.g., a first oligonucleotide, a second oligonucleotide, and a third oligonucleotide), and a second container and/or kit containing a control nucleic acid sample including the first target polynucleotide strand. In some embodiments, the oligonucleotides and/or other components included in the kit may be lyophilized or otherwise stabilized for storage and/or shipment, and reconstituted as desired by the user. Instructions for use can also be included.

### ILLUSTRATIVE EMBODIMENTS

Thus, in some embodiments, this disclosure provides mixtures comprising: a) a first oligonucleotide configured to hybridize to a first sequence in a first target polynucleotide strand, wherein the first sequence includes a target variant nucleotide, and wherein the first oligonucleotide further has a nucleotide residue at its 3′-end that is positioned to hybridize to the target variant nucleotide; b) a second oligonucleotide having a sequence configured to hybridize to a sequence complementary to a second sequence within the first target polynucleotide strand, wherein the second sequence of the first target polynucleotide strand is located 5′ upstream from the first sequence of the first target polynucleotide strand; and, c) a third oligonucleotide having a sequence configured to hybridize to a sequence complementary to a third sequence within the first target polynucleotide strand, wherein the third sequence of the first target polynucleotide strand overlaps at least partially with the first sequence of the first target polynucleotide strand and the third sequence includes the target variant nucleotide. In some embodiments, this disclosure provides mixtures comprising: a) a first oligonucleotide configured to hybridize to a first sequence (A) present within a first target polynucleotide strand, wherein the first sequence includes a target variant nucleotide (“first variant nucleotide”), and wherein the first oligonucleotide further has a nucleotide at its 3′-end that is positioned to hybridize to the first variant nucleotide; b) a second oligonucleotide configured to hybridize to a second sequence (B), where the second sequence is complementary to a third sequence (C), the third sequence being present within the first target polynucleotide strand, wherein the third sequence (C) is located 5′ upstream from the first sequence (A) of the first target polynucleotide strand; and, c) a third oligonucleotide configured to hybridize to a fourth sequence (D) complementary to a fifth sequence (E), the fifth sequence present in the first target polynucleotide strand, wherein the fifth sequence (E) overlaps at least partially with the first sequence (A) in the first target polynucleotide strand and includes the first target variant nucleotide; as well as methods for using the same and kits that can include instructions for using and/or storing such reagents and/or for carrying out such methods. An illustrative embodiment is illustrated in FIG. 1. In some embodiments, the first target polynucleotide strand is a single-stranded polynucleotide molecule including the first target polynucleotide strand. In some embodiments, sequences A, E and C as described herein are located within a single-stranded B polynucleotide molecule on the first target polynucleotide strand.

In some embodiments, at least one additional set of oligonucleotides suitable for amplification and detection of a second target polynucleotide may be included. The oligonucleotides of this at least one additional set of oligonucleotides correspond in function, but not nucleotide sequence, to those used to amplify and detect the first target polynucleotide as described above. For instance, the at least one additional set of oligonucleotides may comprise: a) a first oligonucleotide configured to hybridize to a first sequence (F) present within a second target polynucleotide strand, wherein the first sequence includes a target variant nucleotide (“second variant nucleotide”), and wherein the first oligonucleotide further has a nucleotide at its 3′-end that is positioned to hybridize to the second variant nucleotide; b) a second oligonucleotide configured to hybridize to a second sequence (G), where the second sequence (G) is complementary to a third sequence (H), the third sequence being present within the second target polynucleotide strand, wherein the third sequence (H) is located 5′ upstream from the first sequence (F) of the second target polynucleotide strand; and, c) a third oligonucleotide configured to hybridize to a fourth sequence (I) complementary to a fifth sequence (J), the fifth sequence present in the second target polynucleotide strand, wherein the fifth sequence (J) overlaps at least partially with the first sequence (F) in the second target polynucleotide strand and includes the second target variant nucleotide. Additional sets of oligonucleotides arranged as described herein may also be included. In some embodiments, the first, second, and/or third oligonucleotides comprise between 10-30 nucleotides. In some embodiments, the first and second oligonucleotides are extendable. In some embodiments, the first and second oligonucleotides are primers. In some embodiments, the target variant nucleotide in the third oligonucleotide is at least 2 nucleotides from a 3′ end or a 5′ end of the third oligonucleotide. In some embodiments, the third oligonucleotide can comprise three to six contiguous nucleotides of the first sequence. In some embodiments, the third oligonucleotide further comprises a sequence of nucleotides of the first target polynucleotide strand that does not overlap with a sequence of nucleotides of the first sequence. In some embodiments, the third oligonucleotide can be non-extendable and/or can be a probe. In some embodiments, the third oligonucleotide comprises a minor groove binder (MGB) moiety that can be located at the 3′-terminal nucleotide of the third oligonucleotide. In some embodiments, the third oligonucleotide can be a hydrolysis probe. In some embodiments, the Tₘ of the first oligonucleotide is within 5° C. of the Tₘ of the second oligonucleotide. In some embodiments, the Tₘ of the first oligonucleotide can be 45 to 60° C. and the Tₘ of the second oligonucleotide can be 45 to 60° C. In some embodiments, the Tₘ of the third oligonucleotide can be at least 5° C. and no more than 25° C. higher than the Tₘ of the first oligonucleotide. In some embodiments, the Tₘ of the third oligonucleotide can be at least 8° C. and no more than 12° C. higher than the Tₘ of the first oligonucleotide.

In some embodiments, the third oligonucleotide comprises a detectable label such as a fluorescent label that can be on a first terminal nucleotide of the third oligonucleotide. In some embodiments, the detectable label can be selected from the group consisting of a DNA-binding dye, reporter dye, fluorescent probe, 6-carboxyfluorescein (FAM™), tetrachlorofluorescein (TET™), 6-Carboxy-4′,5′-Dichloro-2′,7′-Dimethoxyfluorescein, Succinimidyl Ester (JOE′), VIC™, a sulfonate derivative of a fluorescein dye with SO₃ instead of the carboxylate group, a phosphoramidite form of fluorescein, a phosphoramidite form of CY5, a non-FRET label, a ferrocene reagent, ABY™, NED™ JUN™ Fluor®488, AlexaFluor®532, AlexaFluor®546, AlexaFluor®594, AlexaFluor®647, AlexaFluor®660, TYE™ 563, TYE™665, TYE™705, and combinations thereof. In some embodiments, the third oligonucleotide can further comprise a quenching moiety (e.g., capable of quenching a signal from the detectable label) that can be on a second terminal nucleotide of the third oligonucleotide. In some embodiments, the quenching label can be selected from the group consisting of tetramethylrhodamine (TAMRA), a non-fluorescent quencher (NFQ), Black Hole Quenchers, Iowa Black, QSY, QSY7, QSY21, NFQ, Dabsyl and/or Dabsyl sulfonate/carboxylate quenchers. In some embodiments, the first terminal nucleotide is the 5′ terminal nucleotide. In some embodiments, the second terminal nucleotide is the 3′ terminal nucleotide.

In some embodiments, the compositions and/or mixtures do not comprise a fourth oligonucleotide. In some embodiments, the mixtures do not comprise a fourth oligonucleotide comprising a detectable label. In some embodiments, the mixture does not comprise a fourth oligonucleotide that binds to the target polynucleotide strand. In some embodiments, the mixture does not comprise a fourth oligonucleotide comprising a detectable label and binds to the target polynucleotide strand. In some embodiments, the third oligonucleotide is the only oligonucleotide in the mixture having a detectable label and a sequence configured to hybridize to a sequence of the target polynucleotide strand.

In some embodiments, this disclosure provides compositions and/or mixtures comprising at least one single-stranded target polynucleotide including the target polynucleotide strand. In some embodiments, the mixtures can comprise at least one double-stranded target polynucleotide including the target polynucleotide strand and a target complement polynucleotide strand, wherein the target complement polynucleotide strand is substantially complementary to the target polynucleotide strand. In some embodiments, the mixtures can comprise at least one double-stranded target polynucleotide including the target polynucleotide strand and a target complement polynucleotide strand, wherein the target complement polynucleotide strand is substantially complementary to the target polynucleotide strand, and a double-stranded target polynucleotide including a variant polynucleotide strand and a variant complement polynucleotide strand, wherein the variant polynucleotide strand has identity to or is substantially identical to the target polynucleotide strand and comprises a different nucleotide at the target variant nucleotide than the target polynucleotide strand, and wherein the variant complement polynucleotide strand is substantially complementary to the variant polynucleotide strand. In some embodiments, a mutant allele can include the target variant nucleotide; can have an identity corresponding to a major allelic sequence or a minor allelic sequence (e.g., an allele having a population frequency of less than 1%); can occur at a position of a single nucleotide polymorphism; can be an allele of, for example, but not limited to, EGFR, KRAS, NRAS, BRAF PIK3CA, AKT1, ESR1, TP53; and/or can be a stochastic mutation. In some embodiments, the mutant allele is either a purine-to-purine single point mutation or a pyrimidine-to-pyrimidine single point mutation at the target variant nucleotide.

In some embodiments, the compositions and/or mixtures can further comprise: a) a fourth oligonucleotide configured to hybridize to a first sequence in a second target polynucleotide strand, wherein the first sequence in the second target polynucleotide comprises a second target variant nucleotide, and wherein the fourth oligonucleotide further comprises a nucleotide at its 3′-end that is positioned to hybridize to the second target variant nucleotide; b) a fifth oligonucleotide comprising a sequence configured to hybridize to a sequence complementary to a second sequence of the second target polynucleotide strand, wherein the second sequence of the second target polynucleotide strand is located 3′ downstream from the first sequence on the second target polynucleotide strand; and, c) a (optionally detectable) sixth oligonucleotide comprising both a detectable label and a sequence configured to hybridize to a sequence complementary to a third sequence of the second target polynucleotide strand, wherein the third sequence of the second target polynucleotide strand overlaps at least partially with the first sequence on the second target polynucleotide strand and comprises the second target variant nucleotide. In some embodiments, the fourth, fifth, and/or sixth oligonucleotide can comprise between 10-40 nucleotides, the fourth and fifth oligonucleotides preferably comprising 10-30 nucleotides. In some embodiments, the fourth and fifth oligonucleotides can be extendable and/or can be primers. In some embodiments, the target variant nucleotide in the sixth oligonucleotide is at least two nucleotides from a 3′ end or a 5′ end of the sixth oligonucleotide. In some embodiments, the sixth oligonucleotide can comprise a detectable label (e.g., a fluorescent label) that may be on the first terminal nucleotide of the sixth oligonucleotide. The sixth oligonucleotide can further comprise a quenching moiety capable of quenching a signal from the detectable label that can be on a second terminal nucleotide of the sixth oligonucleotide. In some embodiments, the sixth oligonucleotide can be non-extendable and/or can be a probe. In some embodiments, the sixth oligonucleotide comprises a minor groove binder (MGB) moiety that can be on the 3′-terminal nucleotide of the sixth oligonucleotide.

In some embodiments, the compositions and/or mixtures can further comprise: a) a fourth oligonucleotide comprising a sequence that is configured to hybridize to a sequence complementary to the first oligonucleotide, wherein the fourth oligonucleotide is configured to substantially hybridize to the first sequence and comprises at its 3′ end a different nucleotide than the complement of the target variant nucleotide; and, b) a (optionally detectable) fifth oligonucleotide configured to hybridize to a sequence complementary to the third oligonucleotide, wherein the fifth oligonucleotide comprises a different nucleotide at the site of the target variant nucleotide. In some embodiments, the fourth and fifth oligonucleotide comprise between 10-40 nucleotides, the fourth oligonucleotide preferably being between 10-30 nucleotides. In some embodiments, the fourth oligonucleotide can be extendable and/or can be a primer. In some embodiments, the target variant nucleotide in the fifth oligonucleotide can be at least two nucleotides from a 3′ end or a 5′ end of the fifth oligonucleotide. In some embodiments, the fifth oligonucleotide can comprise a detectable label (e.g., a fluorescent label) that can be on a first terminal nucleotide, which can be distinguishable from the detectable label on the third oligonucleotide. The fifth oligonucleotide can further comprise a quenching moiety capable of quenching a signal from the detectable label that can be on a second terminal nucleotide of the third oligonucleotide. In some embodiments, the fifth oligonucleotide is non-extendable and/or is a probe. In some embodiments, the fifth oligonucleotide comprises a minor groove binder (MGB) moiety that can be on the 3′-terminal nucleotide thereof.

In some embodiments, this disclosure provides compositions and/or mixtures comprising a plurality of oligonucleotide sets, wherein each oligonucleotide set comprises: a) a first oligonucleotide configured to hybridize to a first sequence in a target polynucleotide strand, wherein the first sequence comprises a target variant nucleotide, and wherein the first oligonucleotide further comprises a nucleotide residue at its 3′-end that is positioned to hybridize to the target variant nucleotide; b) a second oligonucleotide comprising a sequence configured to hybridize to a sequence that is complementary to a second sequence within the first target polynucleotide strand, wherein the second sequence is located 5′ upstream from the first sequence; and, c) a detectable third oligonucleotide comprising a sequence configured to hybridize to a sequence that is complementary to a third sequence of the target polynucleotide strand, wherein the third sequence overlaps at least partially with the first sequence and comprises the target variant nucleotide; wherein the first oligonucleotide of each set is configured to hybridize to a different first sequence, the third oligonucleotide of each set shares sequence similarity to a different third sequence, and, wherein the third oligonucleotide of each set comprises a different and distinguishable detectable label.

In some embodiments, this disclosure provides compositions and/or mixtures comprising a plurality of oligonucleotide sets, wherein each oligonucleotide set comprises: a) a first oligonucleotide configured to hybridize to a first sequence in a first target polynucleotide strand, wherein the first sequence includes a target variant nucleotide, and wherein the first oligonucleotide further has a nucleotide residue at its 3′-end that is positioned to hybridize to the target variant nucleotide; b) a second oligonucleotide having a sequence configured to hybridize to a sequence complementary to a second sequence within the first target polynucleotide strand, wherein the second sequence of the first target polynucleotide strand is located 5′ upstream from the first sequence of the first target polynucleotide strand; and, c) a third oligonucleotide having a sequence configured to hybridize to a sequence complementary to a third sequence within the first target polynucleotide strand, wherein the third sequence of the first target polynucleotide strand overlaps at least partially with the first sequence of the first target polynucleotide strand and the third sequence includes the target variant nucleotide; wherein the first oligonucleotide of each set is configured to hybridize to a different first sequence, the third oligonucleotide of each set shares sequence similarity to a different third sequence, and/or, the third oligonucleotide of each set comprises a different and distinguishable detectable label.

In some embodiments, this disclosure provides compositions and/or mixtures comprising a plurality of oligonucleotide sets, wherein each oligonucleotide set comprises: a) a first oligonucleotide configured to hybridize to a first sequence (A) present within a first target polynucleotide strand, wherein the first sequence includes a target variant nucleotide (“first variant nucleotide”), and wherein the first oligonucleotide further has a nucleotide at its 3′-end that is positioned to hybridize to the first variant nucleotide; b) a second oligonucleotide configured to hybridize to a second sequence (B), where the second sequence is complementary to a third sequence (C), the third sequence present within the first target polynucleotide strand, wherein the third sequence (C) is located 5′ upstream from the first sequence (A) of the first target polynucleotide strand; and, c) a third oligonucleotide configured to hybridize to a fourth sequence (D) complementary to a fifth sequence (E), the fifth sequence present in the first target polynucleotide strand, wherein the fifth sequence (E) overlaps at least partially with the first sequence (A) in the first target polynucleotide strand and includes the first target variant nucleotide; wherein the first oligonucleotide of each set is configured to hybridize to a different first sequence, the third oligonucleotide of each set shares sequence similarity to a different third sequence, and/or the third oligonucleotide of each set comprises a different and distinguishable detectable label.

In some embodiments, the compositions and/or mixtures of this disclosure can comprise additional components. For instance, the mixtures can further comprise between about 10 mM and about 80 mM potassium chloride, and/or between about 10 mM and about 40 mM ammonium sulfate. In some embodiments, the mixture can comprise a concentration of potassium chloride between 30 mM and 80 mM; and, a concentration of ammonium sulfate between 10 mM and 40 mM. In some embodiments, the mixtures can comprise about 45 mM potassium chloride about 22 mM ammonium sulfate. In some embodiments, the mixtures can further comprise a polymerase that can be thermostable, and can further comprise a hot start component such as, but not limited to, an antibody directed to the thermostable polymerase; an oligonucleotide; and/or an aptamer. In some embodiments, the mixtures can further comprise a source of nucleotides. In some embodiments, the mixtures are reaction mixtures. In some other embodiments, the mixtures are storage mixtures. In some embodiments, the mixtures can comprise a nucleic acid sample suspected of comprising the target polynucleotide strand. In some embodiments, the mixtures can include a master mix and/or be a master mix. In some embodiments, the mixtures can further comprise an amplicon comprising the first sequence of the target polynucleotide strand. In some embodiments, the mixture does not include an amplicon that includes a sequence of a second polynucleotide strand. In some embodiments, this disclosure provides mixtures comprising one or more of at least one detergent, glycerol, at least one reference dye, bovine serum albumin, and/or gelatin. In some embodiments, the mixtures can be lyophilized.

In some embodiments, this disclosure provides kits comprising a first container containing one or more mixtures described herein, and a second container containing a control nucleic acid sample including sharing sequence similarity with the first target polynucleotide strand. In some embodiments, the control nucleic acid sample can only include a portion of the target polynucleotide (e.g., a xeno sequence) and, in some embodiments, the control nucleic acid sample includes the entire first target polynucleotide strand.

This disclosure also provides method for detecting a target polynucleotide including a target variant nucleotide using the reagents (e.g., oligonucleotides) described herein. In some embodiments, the methods can comprise: a) forming a reaction mixture of a test nucleic acid sample and one or more of the mixtures described herein; b) carrying out an amplification reaction using at least the first and second oligonucleotides as primers to produce amplicons of a target polynucleotide sequence of the target polynucleotide if present in the sample; and, c) detecting the amplicons by detecting a change in a detectable property of the third oligonucleotide; wherein detecting amplicons in step c) indicates the target polynucleotide is present within the test nucleic acid sample. In some embodiments, the target polynucleotide can be identified in a sample comprising a mixture of nucleic acids comprising the target polynucleotide (e.g., rare/low abundance nucleic acids) and non-target polynucleotides (e.g., high abundance nucleic acids) that do not include the target variant nucleotide.

In some embodiments, this disclosure provides methods for detecting a target polynucleotide molecule including a target variant nucleotide in a test polynucleotide sample, the method comprising: a) forming a reaction mixture of a test polynucleotide sample and a mixture of the first, second and third oligonucleotides described herein (in some embodiments, also including the fourth, fifth and/or sixth oligonucleotides described herein); b) carrying out an amplification reaction using at least the first and second oligonucleotides as primers to produce amplicons of a target polynucleotide sequence of the target polynucleotide molecule if present in the test polynucleotide sample; and, c) detecting the amplicons produced in step b) by detecting a change in a detectable property of the third oligonucleotide; wherein detecting amplicons in step c) indicates the target polynucleotide molecule is present within the test polynucleotide sample. In some embodiments of these methods, the target polynucleotide molecule is detected in a test polynucleotide sample comprising a mixture of polynucleotide molecules, the mixture including polynucleotide molecules that include a first variant form of the target variant nucleotide (“first variant target polynucleotide molecules”) and polynucleotide molecules that include a second variant form of the target variant nucleotide (“second variant target polynucleotide molecules”). In some embodiments of these methods, the test polynucleotide sample comprises polynucleotide strands that do not include a target polynucleotide sequence (“non-target polynucleotide molecules”). In some embodiments of these methods, the test sample comprises more non-target polynucleotide molecules than target polynucleotide molecules. In some embodiments of these methods, the target polynucleotide molecules are rare allelic or mutant polynucleotide sequences. In some embodiments of these methods, the non-target polynucleotide molecules are major allelic or wild-type polynucleotide sequences. In some embodiments of these methods, the test sample comprises less than 2%, less than 1%, less than 0.1%, less than 0.01%, or less than 0.001% target polynucleotide molecules relative to non-target polynucleotide molecules. In some embodiments of these methods, the test sample comprises less than 2%, less than 1%, less than 0.1%, less than 0.01%, or less than 0.001% of second variant target polynucleotide molecules relative to first variant target polynucleotide molecules. In some embodiments of these methods, the first variant target polynucleotide molecules and/or second variant target polynucleotide molecules are mutant polynucleotide sequences. In some embodiments of these methods, the first variant target polynucleotide molecules and/or second variant target polynucleotide molecules are wild-type polynucleotide sequences.

In some embodiments, the method further comprises enriching the number of first variant polynucleotide molecules in the polynucleotide test sample relative to the second variant polynucleotide molecules prior to steps a) through c) (an “enrichment” step). In some embodiments of these methods, the enrichment step comprises an amplification reaction under conditions that differ from those used in steps a) through c). In some embodiments, the enrichment step increases (e.g., enriches) the number of first variant polynucleotide molecules in the polynucleotide test sample by at least two-fold, four-fold, six-fold, eight-fold, or ten-fold relative to the second variant polynucleotide molecules. For instance, in some embodiments, the enriching step is carried out using PCR comprising from 15-25 cycles at a temperature of 12-16 degrees higher than the calculated melting temperature (T_{M}) of the first oligonucleotide. In some embodiments, steps a) through c) comprise 40 cycles near the Tₘ of target site-specific probe (e.g., third oligonucleotide) at four to six degrees below the temperature at which enrichment is carried out. In some embodiments of these methods, the test polynucleotide sample is derived from a mammalian or non-mammalian animal tissue or cell, or a plant tissue or cell. In some embodiments of these methods, the sample is selected from the group consisting of saliva, cheek tissue, skin, hair, blood, plasma, urine, feces, semen, and a tumor sample. In some embodiments of these methods, the polynucleotide test sample is derived from a cancer cell. In some embodiments of these methods, the detection of amplicons indicates the presence of cancer cells within a tissue from which the test polynucleotide sample was derived. In some embodiments of these methods, the target polynucleotide includes at least one mutation in EFGR (e.g., FIG. 20), Ras (e.g., at least one KRAS or NRAS mutation (e.g., FIG. 21)), Kit, pTEN, and/or p53; and/or at least one KRAS or NRAS mutation; and/or at least one mutation listed in Table 1 and/or Table 2. In some embodiments of these methods, the amplification reaction is a polymerase chain reaction (PCR) such as real-time PCR. In some embodiments of these methods, the third oligonucleotide has a Tₘ 6-20° C. (optimally 8-12° C.) above the Tₘ of the first oligonucleotide and the PCR is carried out with an annealing temperature within 5° C. of the Tₘ of the first oligonucleotide. In some embodiments, these methods are carried out using a kit that includes a first container containing at least the first oligonucleotide, the second oligonucleotide, and the third oligonucleotide and a second container containing a control polynucleotide sample including the first target polynucleotide strand. In some embodiments, the method detects a target variant nucleotide indicative of a mutation, and wherein the polynucleotide sample comprises 1-10 copies of the target polynucleotide. In some embodiments of these methods, the target variant nucleotide includes a purine base and a corresponding major allelic or wild-type nucleotide at the target variant nucleotide position includes a different purine base; or the target variant nucleotide includes a pyrimidine base and a corresponding major allelic or wild-type nucleotide at the target variant nucleotide position includes a different pyrimidine base.

In some embodiments, the methods can comprise further comprising enriching the number of target polynucleotides in the sample relative to wild-type nucleic acids prior to steps a) through c). In some embodiments, this enrichment process can comprise an amplification reaction comprising different conditions as used in steps a) through c). For instance, in some embodiments, the enrichment process can be carried out using polymerase chain reaction comprising from 15-25 cycles at a temperature of 12-16° C. higher than the calculated melting temperature (Tₘ) of the first oligonucleotide. In some embodiments, steps a) through c) can comprise 40 cycles near the Tₘ of third oligonucleotide and four to six degrees below the temperature at which enrichment was carried out (e.g., where “near” can mean 4-6° C. below the temperature used in the enrichment process). In some embodiments, the test nucleic acid sample is derived from a mammalian or non-mammalian animal tissue or cell, or a plant tissue or cell (e.g., saliva, cheek tissue, skin, hair, blood, plasma, urine, feces, semen, and a tumor sample (e.g., a cancer cell)). In some embodiments, the detection of amplicons indicates the presence of cancer cells within a tissue from which the test nucleic acid sample was derived. In some embodiments, the target polynucleotide includes at least one mutation in EFGR (e.g., FIG. 20), Ras (e.g., at least one KRAS or NRAS mutation (e.g., FIG. 21)), BRAF, Kit, pTEN, ESR1, and/or p53; and/or one or more of the mutations listed in Table 1 and Table 2. In some embodiments, the amplification reaction is a polymerase chain reaction (PCR) such as, but not limited to, real-time PCR or quantitative PCR (qPCR). In some embodiments, for example, the third oligonucleotide can have a Tₘ 6-20° C. (optimally 8-12° C.) above the Tₘ of the first oligonucleotide and the PCR can be carried out with an annealing temperature within 5° C. of the Tₘ of the first oligonucleotide.

In some embodiments, the methods can be carried out using a kit that includes a first container containing at least the first oligonucleotide, the second oligonucleotide, and the third oligonucleotide and a second container containing a control nucleic acid sample including the first target polynucleotide strand. In some embodiments, the methods can detect a target variant nucleotide indicative of a mutation, and wherein the nucleic acid sample comprises 1-10 copies of the target polynucleotide sequence from a background of a much larger number of wild-type nucleic acid sequences. In some embodiments, the target variant nucleotide includes a purine base and a corresponding wild-type nucleotide at the target variant nucleotide position includes a different purine base; and/or a pyrimidine base and a corresponding wild-type nucleotide at the target variant nucleotide position includes a different pyrimidine base. Other methods are also contemplated by this disclosure as would be understood by those of ordinary skill in the art.

General information

The terms “about”, “approximately”, and the like, when preceding a list of numerical values or range, refer to each individual value in the list or range independently as if each individual value in the list or range was immediately preceded by that term. The terms mean that the values to which the same refer are exactly, close to, or similar thereto.

As used herein, a subject or a host is meant to be an individual. The subject can include domesticated animals, such as cats and dogs, livestock (e.g., cattle, horses, pigs, sheep, and goats), laboratory animals (e.g., mice, rabbits, rats, guinea pigs) and birds. In one aspect, the subject is a mammal such as a primate or a human.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different polynucleotides or may not include a combination such that the description includes both the combination and the absence of the combination (i.e., individual members of the combination).

Ranges may be expressed herein as from about one particular value, and/or to about another particular value. When such a range is expressed, another aspect includes from the one particular value and/or to the other particular value. Similarly, when values are expressed as approximations, by use of the antecedent about or approximately, it will be understood that the particular value forms another aspect. It will be further understood that the endpoints of each of the ranges are significant both in relation to the other endpoint, and independently of the other endpoint. Ranges (e.g., 90-100%) are meant to include the range per se as well as each independent value within the range as if each value was individually listed.

The term “combined” or “in combination” or “in conjunction” may refer to a physical combination of agents that are administered together or the use of two or more agents in a regimen (e.g., administered separately, physically and/or in time) for treating, preventing and/or ameliorating a particular disease.

When the terms treat, prevent, and/or ameliorate or derivatives thereof are used herein in connection with a given treatment for a given condition (e.g., preventing cancer infection by HIV), it is meant to convey that the treated patient either does not develop a clinically observable level of the condition at all, or develops it more slowly and/or to a lesser degree than he/she would have absent the treatment. These terms are not limited solely to a situation in which the patient experiences no aspect of the condition whatsoever. For example, a treatment will be said to have prevented the condition if it is given during exposure of a patient to a stimulus that would have been expected to produce a given manifestation of the condition, and results in the patient's experiencing fewer and/or milder symptoms of the condition than otherwise expected. For instance, a treatment can “prevent” infection by resulting in the patient's displaying only mild overt symptoms of the infection; it does not imply that there must have been no penetration of any cell by the infecting microorganism.

Similarly, reduce, reducing, and reduction as used herein in connection with prevention, treatment and/or amelioration of a given condition by a particular treatment typically refers to a subject developing an infection more slowly or to a lesser degree as compared to a control or basal level of developing an infection in the absence of a treatment. A reduction in the risk of infection may result in the patient's displaying only mild overt symptoms of the infection or delayed symptoms of infection; it does not imply that there must have been no penetration of any cell by the infecting microorganism.

All references cited within this disclosure are hereby incorporated by reference in their entirety. Certain embodiments are further described in the following examples. These embodiments are provided as examples only and are not intended to limit the scope of the claims in any way.

### EXAMPLES

The reagents and assay designs disclosed herein exemplify detection of <10 copies (e.g., as low as one to three copies) of a target nucleotide sequence (e.g., a mutant, rare or low abundance target nucleotide sequence) from a background of non-target nucleotide sequences (e.g., “wild-type”, common, or higher abundance nucleotide sequences). As discussed above, these methods may be useful in the detection of such rare (e.g., low-abundance) target nucleic acids in a variety of assays, including but not limited to cell-free DNA cancer-related assays (e.g., for initial diagnosis and/or detection of recurrences), single nucleotide polymorphism (SNP) determination assays, in a forensic-related assay, and/or in an agricultural-related assays.

As shown in these examples, quantitative polymerase chain reaction (qPCR) reactions were performed to detect, identify and/or quantify low abundance nucleic acid sequences (e.g., representing mutated genes or allelic variants) in the presence of more abundant wild-type nucleic acid sequences (e.g., representing non-mutated genes or major alleles) using exemplary primer and probe assay designs such as that shown in FIG. 1. Each qPCR reaction utilized a mixture of at least one forward primer (e.g., first oligonucleotide), at least one reverse primer (e.g., second oligonucleotide), and at least one probe (e.g., third oligonucleotide, which may be a 5′ nuclease or TaqMan probe), as exemplified without limitation in the examples described below that are used to amplify a target polynucleotide comprising first and second target polynucleotide strands. One of the primers (i.e., either the forward or reverse primer; the first oligonucleotide) had binding specificity for a first nucleotide sequence (sometimes referred to as the “first sequence”) including at least one target variant nucleotide (a target site for example, that can be indicative of a mutant gene or a low abundance allelic variant) in a first target polynucleotide strand. The target variant nucleotide was corresponded to the terminal nucleotide of the primer, such that the binding specificity of the primer was largely determined by the target variant nucleotide (i.e., was hybridizable thereto, meaning it was the same as or complementary thereto). Thus, a first oligonucleotide, either the forward or reverse primer, was designed to bind to the first nucleotide sequence and to include at its 3′ end a nucleotide complementary to the target variant nucleotide (e.g., a nucleotide corresponding to a mutant gene or allelic variant). In some embodiments, the nucleotide complementary to the target variant nucleotide at the 3′ end of the first oligonucleotide may be within two nucleotides (n+2, n+1, n) of the actual 3′ terminal nucleotide (n) of the first oligonucleotide.

A second oligonucleotide (i.e., the other primer; forward or reverse primer) and a third oligonucleotide (e.g., a target site-specific probe, such as a TaqMan™ probe or a hydrolysis probe), each having a sequence that shares sequence identity with a second sequence and a third sequence, respectively, of the first polynucleotide strand (i.e., the second and third sequences are typically complementary to a second polynucleotide strand of a double-stranded target polynucleotide). The second oligonucleotide (e.g., second primer) in the mixture was designed to share identity with the first target polynucleotide strand, but at a position upstream or downstream of, and not overlapping, the first sequence to which the first oligonucleotide (e.g., first primer) was hybridized. The third sequence to which the third oligonucleotide (e.g., the TaqMan™ probe) was hybridized was designed to at least partially overlap the first sequence and include the target variant nucleotide. In some exemplary embodiments, the third oligonucleotide (e.g., the TaqMan™ probe) also included additional nucleotides identical to the first target polynucleotide strand that did not overlap the first sequence bound by the first oligonucleotide or first primer. In some exemplary embodiments, the third oligonucleotide (e.g., target site-specific probe, such as a TaqMan™ probe) used in these experiments included an oligonucleotide having a fluorophore (e.g. 6-FAM) covalently bound at its 5′ end, a quencher (sometimes referred to as a non-fluorescent quencher; “NFQ”), and a minor groove binder (“MGB”) covalently bound to its 3′ end. Not to be limited by theory, it is believed that the minor groove binder bound to the 3′ end of the oligonucleotide renders it non-extendable by a Taq polymerase. In general, the third oligonucleotide (e.g., TaqMan™ probe) was designed to exhibit a melting temperature (T_{M}) of 6-20° C. (optimally 8-12° C.) above that of the first oligonucleotide (e.g., first primer) and PCR was carried out using standard techniques (e.g., 40 cycles with an annealing/extension temperature near the T_{M} of the third oligonucleotide (e.g., target site-specific probe, such as the TaqMan probe). For instance, in exemplary assays regarding KRAS G12R, the Tₘ (as calculated in Primer Express) first oligonucleotide was 52.4° C., the Tₘ of the second oligonucleotide was 50.8° C., and the Tₘ of the third oligonucleotide was 61° C. In another example, in assays regarding KRAS G12C, the Tₘ of the first oligonucleotide was 50.2° C., the Tₘ of the second oligonucleotide was 51.6° C., and the Tₘ of the third oligonucleotide was 62° C. In yet another example regarding KRAS G12A, the Tₘ of the first oligonucleotide was 51.4° C., the Tₘ of the second oligonucleotide was 51.6° C., and the Tₘ of the third oligonucleotide was 61° C.

In certain embodiments, the qPCR reactions included an “enrichment cycle” or “enrichment phase” in which low abundance nucleic acids were amplified in preference to high abundance (e.g., wild-type) nucleic acids, the mixtures being subjected to 1 cycle of 95° C. for 2 min; 15-20 cycles of 95° C. for 1 sec and 64° C. for 20 sec (enrichment phase); and 40 cycles of 95° C. for 1 sec and, 60° C., for 20 sec (amplification and detection phase). In some embodiments, qPCR reactions were performed without an enrichment phase, and the mixtures subjected to: 1 cycle of 95° C. for 2 min; and 40 cycles of 95° C. for 1 sec and, 60° C. for 20 sec (amplification and detection phase). When reactions contained VIC labeled RPPH1 assay, the data generated from the amplification reactions was exported in Excel format, the Cq values for replicate reactions averaged and the delta average Cq of FAM and VIC targets in each condition determined and plotted, and the delta Cq was used a quantification method.

In certain illustrative examples below, for instance, the target variant nucleotide is present within the allelic variant DNA, such as for KRAS, BRAF, EGFR, etc. (which may be referred to elsewhere herein as the target nucleic acid); the forward or reverse primer (the first oligonucleotide) was designed to bind the target variant nucleotide by including a nucleotide at the 3′ end complementary to the target variant nucleotide of the allelic variant KRAS, BRAF, EGFR, etc. DNA being assayed; the labeled third oligonucleotide (which may be a TaqMan probe) was designed to include the target variant nucleotide; and the target nucleic acid was amplified and detected. Additional details of the various assays carried out in these illustrative examples are provided below.

### Example 1

### Differentiation of Wild-Type and Mutant Sequences

A. qPCR Assay

For qPCR analysis, samples of wild-type DNA (Control DNA from CEPH Individual 1347-02 (“CEPH”); Thermo Fisher Scientific Cat. No. 403062) were spiked with or without 0.1% of a target allelic variant KRAS DNA (KRAS Reference Standards DNA purchased from Horizon Discovery Ltd; Cat. Nos. HD287 for KRAS G12R; HD264 for KRAS G12A). Samples of wild-type DNA (10 ng) or the spiked samples (10 ng wild-type (CEPH) DNA with allelic variant spike (e.g., mutant sample) were added to 300 nM each primer (forward and reverse; first and second oligonucleotides), 250 nM probe (third oligonucleotide), 1 mM dNTPs, 39 mM Tris pH 8, 2.55 mM MgCl₂, 30 mM KCl, 16 mM (NH₄)₂SO₄, 0.1 mg/mL BSA, 7% glycerol and 0.085U/uL Platinum Taq to form a reaction mixture and 10 μl aliquots of the mixtures were plated in 4 replicate wells of a 96-well plate. The qPCR reaction was performed on a QuantStudio 5 F96 (Thermo Fisher Scientific, Waltham, Mass.). For qPCR reactions performed without an enrichment phase, the mixtures were subjected to: 1 cycle of 95° C. for 2 min; and 40 cycles of 95° C. for 1 sec and then between 58 to 62° C., as indicated, for 20 sec (amplification and detection phase). For qPCR reactions performed with an enrichment phase, the mixtures were subjected to 1 cycle of 95° C. for 2 min; 19 cycles of 95° C. for 3 sec and 64° C. for 20 sec (enrichment) and 40 cycles of 95° C. for 1 sec and then between 58 to 62° C., as indicated, for 20 sec (amplification and detection phase). The primers, probes, and target nucleic acids utilized in these assays were designed as described above. The data was exported in Excel format, the Cq values for replicate reactions averaged and the delta average Cq of targets determined and plotted. The delta Cq was used a quantification method.

### B. Experimental Results

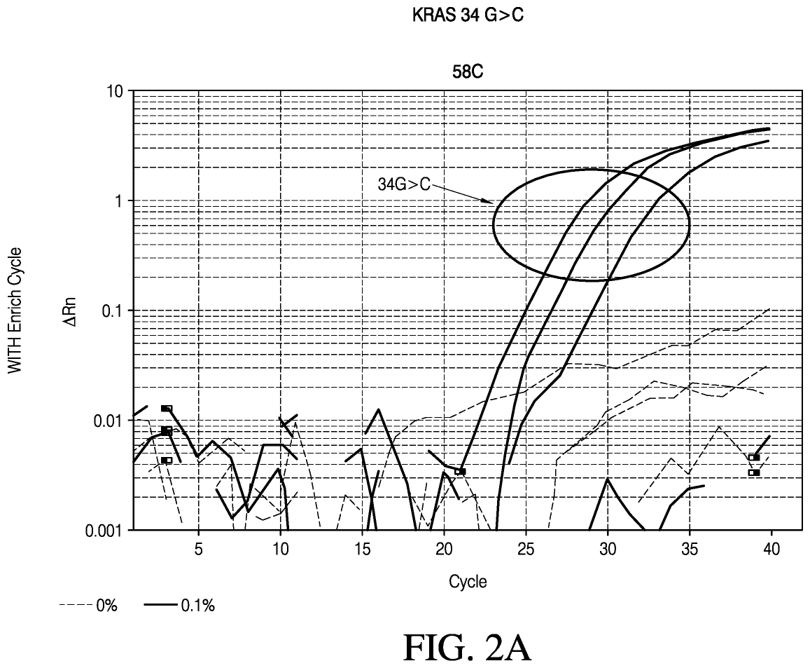

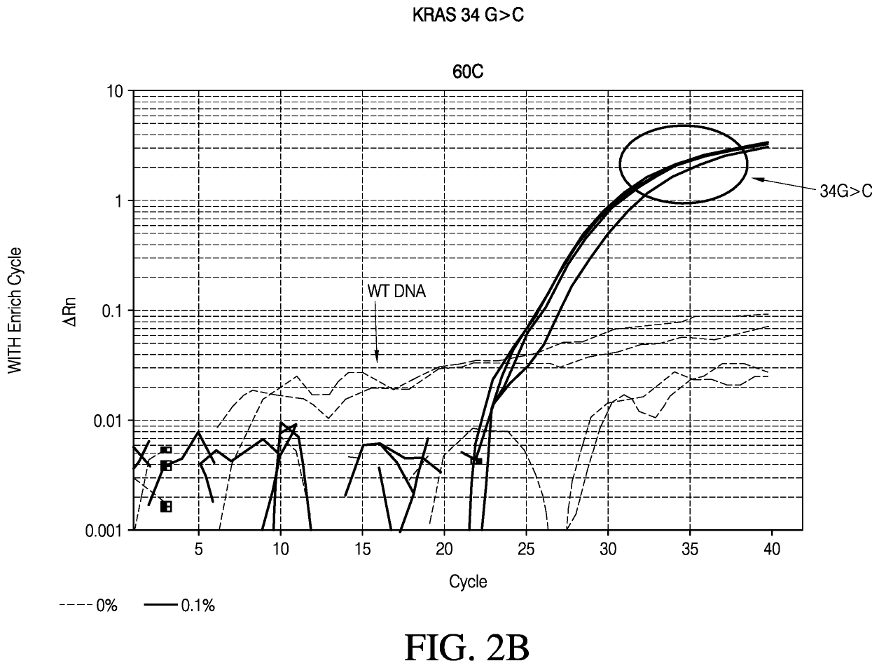

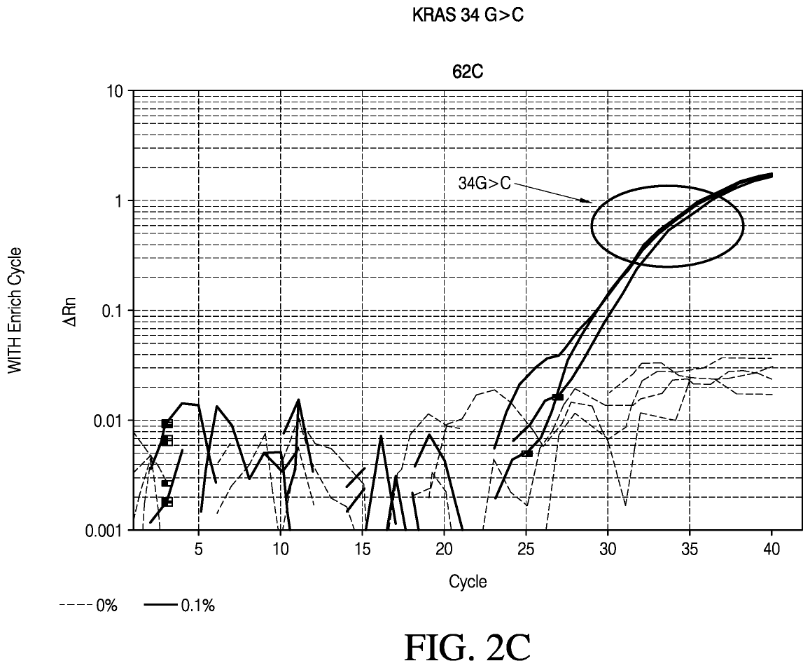

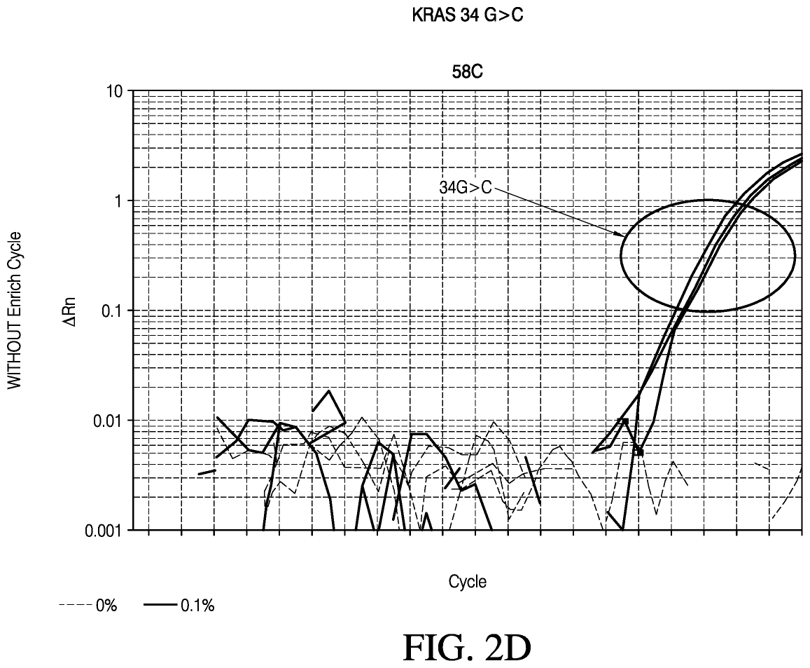

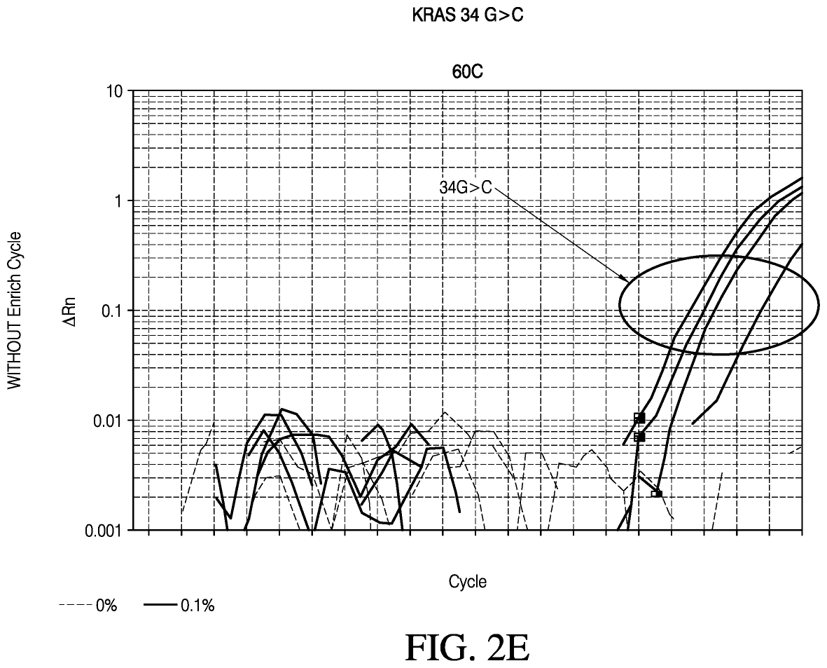

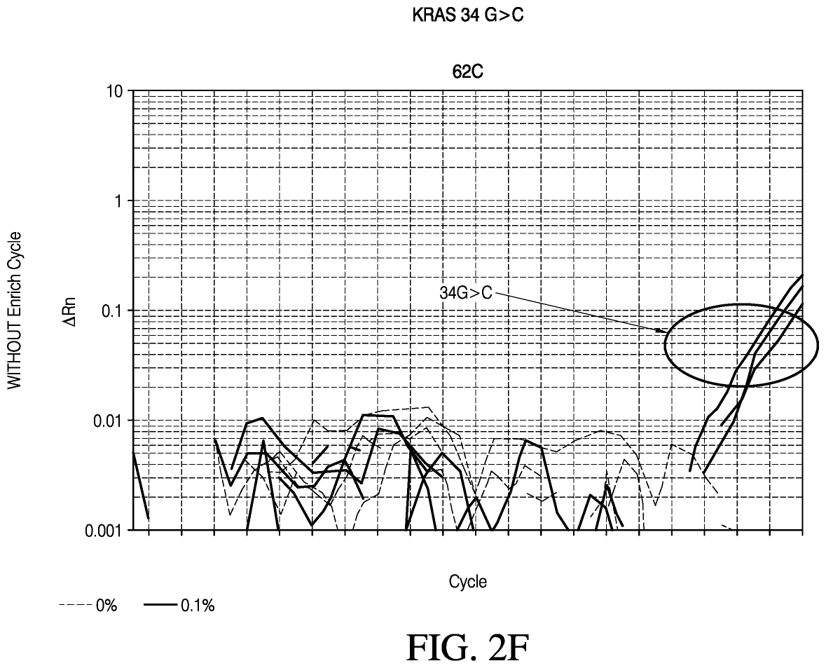

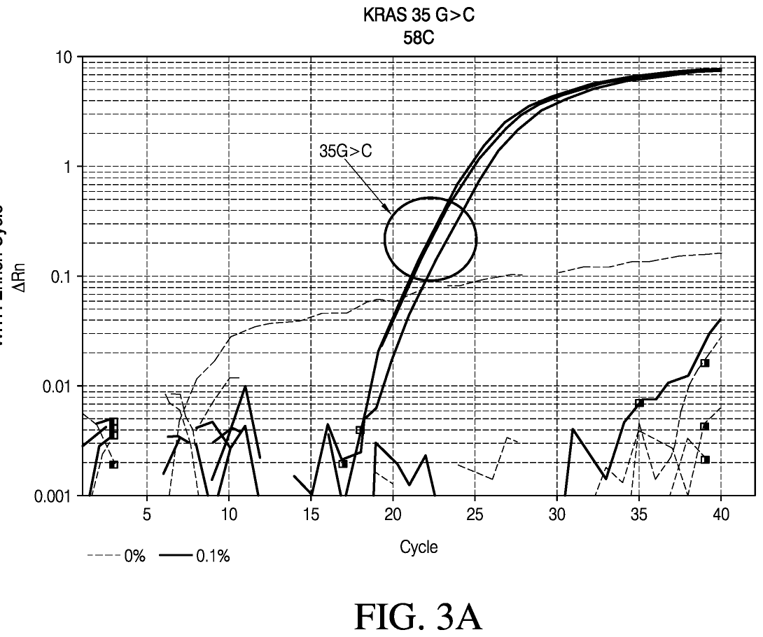

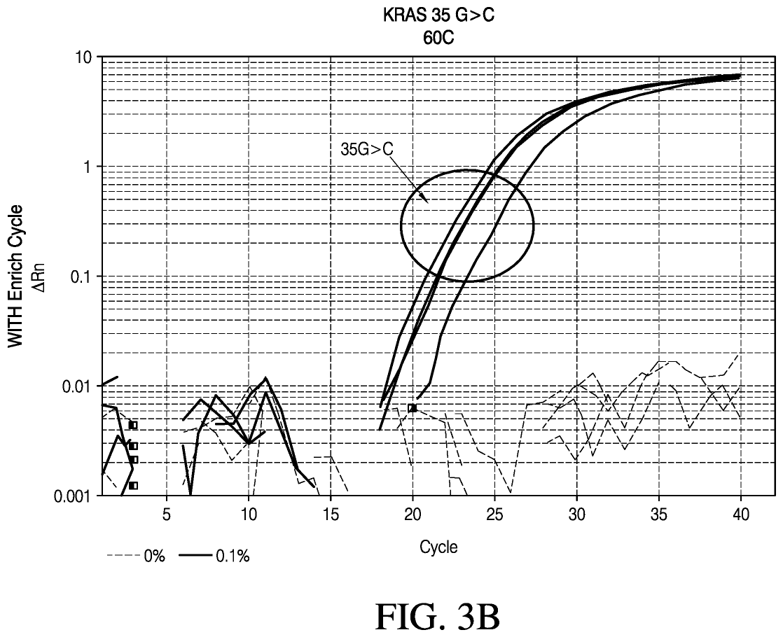

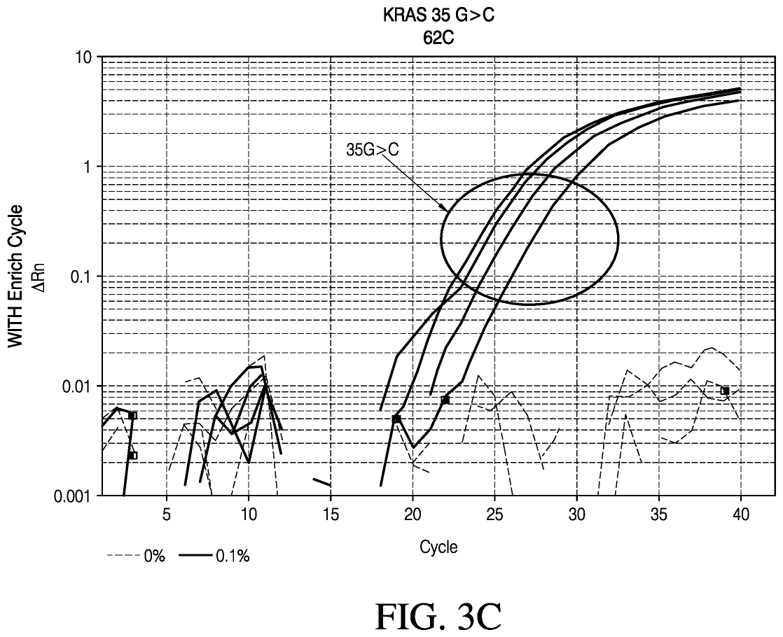

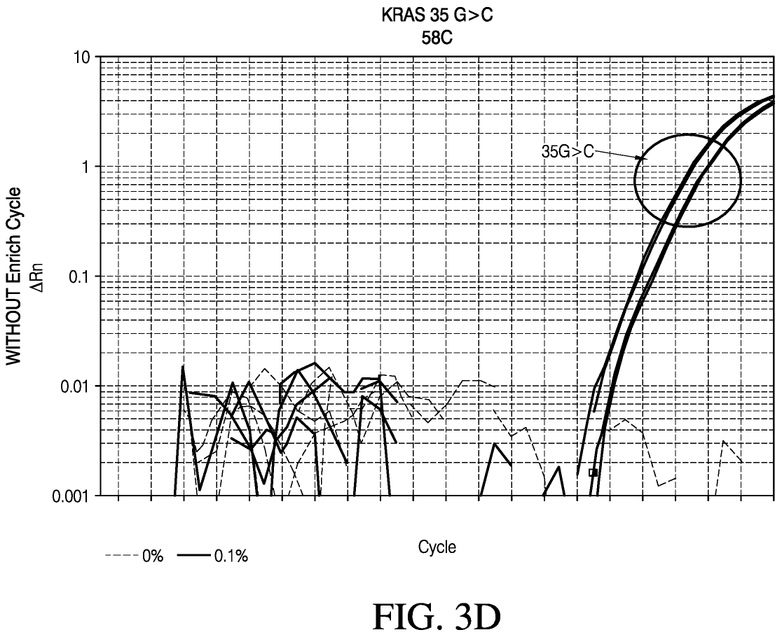

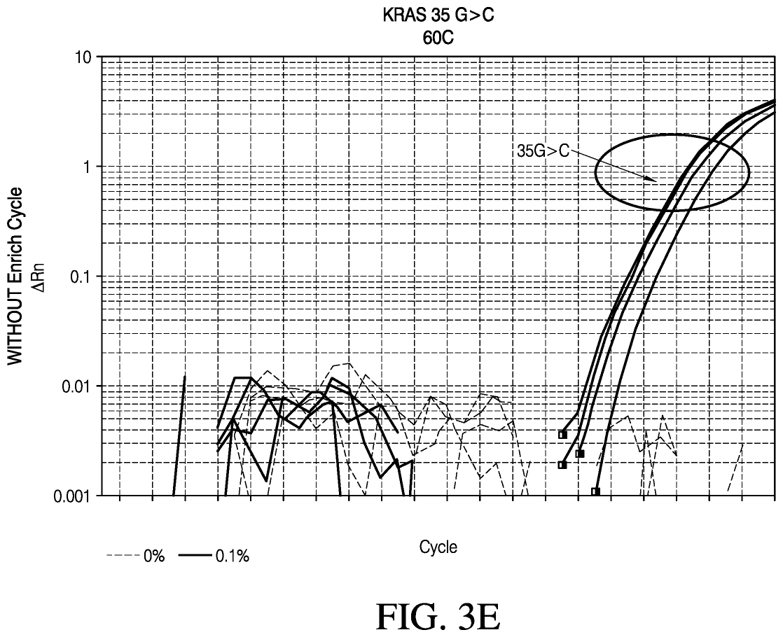

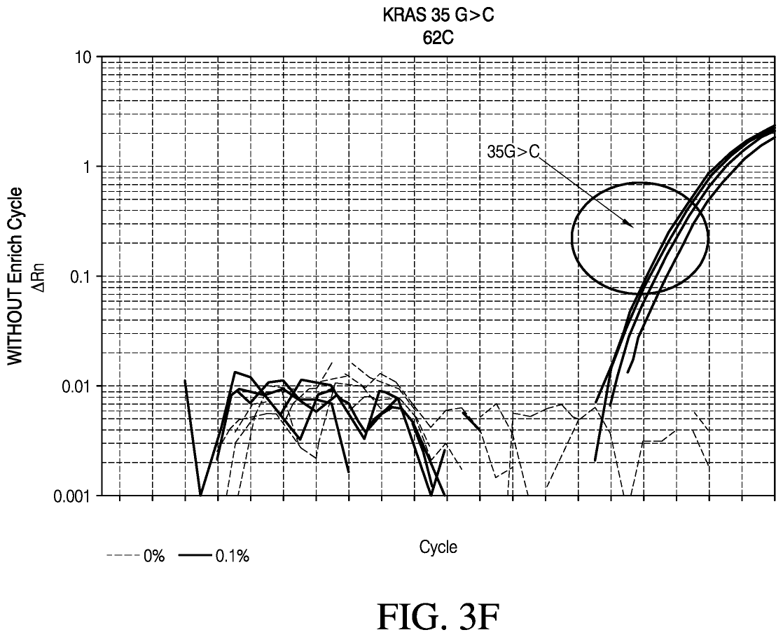

qPCR reactions with an assay design wherein the reactions contained a target site-specific probe and two primers, as described above, one of which was a first oligonucleotide (e.g., target-site specific primer) and the other of which was a second oligonucleotide (e.g., locus-specific primer) discriminately amplified low abundance target nucleic acids in the presence of highly similar and abundant nucleic acids (FIGS. 2-4). Notably, the assay design was able to discriminate between samples with and without spiked allelic variant KRAS DNA with or without an amplification enrichment phase (results in experiments including enrichment phase illustrated in FIGS. 2A-C and 3A-C). Under these reaction conditions and assays, amplification curves indicative of successful amplification were only observed when the low abundance allelic variant KRAS DNA (10 ng wild-type (CEPH) DNA plus 10 pg target sequence (0.1% or three copies)) was present in a reaction mixture (i.e., and not when only wild-type DNA (10 ng wild-type (CEPH) DNA) was present) (FIGS. 2A-F and 3A-F).

Reactions that included an enrichment phase had lower Ct values, indicating that the enrichment phase cycles preferentially if not exclusively amplified low abundance allelic variant DNA, thus further improving performance using the assay design (FIGS. 2A-F and 3A-F). While the background fluorescence of some samples containing only wild-type KRAS DNA was increased with inclusion of the enrichment phase. These background qPCR curves were easily distinguishable from typical qPCR curves as seen when low abundance allelic variants were present (e.g., FIGS. 2A-C and 3A). Not to be limited by theory, it is believed that the elevated temperature in the enrichment phase favors annealing of the target sequence-specific primers to the low abundance target nucleic acids, which form a full match when bound to the target sequence-specific primer, as compared to annealing of the target sequence-specific primers to the abundant nucleic acids, which contain a single base mismatch when bound to the target sequence-specific primer, which subsequently leads to preferential amplification of the low abundance target nucleic acids over the abundant nucleic acids.

In subsequent experiments, a similar assay design was used to discriminate wild-type DNA from wild-type DNA individually spiked with 0.1% of the following allelic variant KRAS DNA as indicated (Horizon Discovery Ltd): 34 G>C (FIG. 4A), 35 G>C (FIG. 4B), 34 G>A (FIG. 4C), 34 G>T (FIG. 4D), 35 G>A (FIG. 4E), and 35 G>T (FIG. 4F). qPCR reactions configured with an assay design wherein the reaction contained a first oligonucleotide (e.g., a target sequence-specific primer), a second oligonucleotide (e.g., locus-specific primer), and a third oligonucleotide (e.g., target site-specific probe) had lower Ct values in samples containing wild-type KRAS DNA samples spiked with 0.1% of the indicated allelic variant KRAS DNA than in samples containing wild-type KRAS DNA alone. Table 3 shows the average Ct value with or without the spiked allelic variant KRAS DNA from these qPCR reactions and shows the difference between the two Ct values (dCt). All of the primer/probe combinations demonstrated effective discrimination of samples containing low abundance KRAS allelic DNA with wild-type KRAS DNA and five of the six allelic variants had dCt values of 9 or higher.

TABLE 3
Ct values for qPCR reaction with wild-
type DNA or wild-type DNA spiked with
0.1% of the indicated allelic variant KRAS DNA

| | Wild-type | 0.1% Allelic Variant | |
|---|---|---|---|
| Assay | Avg. Ct Value | Avg. Ct Value | dCt |
| 35 G > C | 40.0 | 23.7 | 16.3 |
| (p. G12A) | | | |
| 34 G > C | 40.0 | 28.1 | 11.9 |
| (p. G12R) | | | |
| 34 G > A | 27.9 | 24.9 | 3.0 |
| (p. G12S) | | | |
| 35 G > T | 38.2 | 23.3 | 14.8 |
| (p. G12V) | | | |
| 34 G > T | 32.3 | 23.3 | 9.0 |
| (p. G12C) | | | |
| 35 G > A | 31.6 | 21.1 | 10.5 |
| (p. G12D) | | | |

Additional experiments using assay designs and cycling conditions similar to those disclosed in this Example were also performed for various other samples and detected 1-3 copies of allelic variant DNA in a background of wild-type target nucleic acids (data not shown).

### Example 2

### Potassium Chloride and Ammonium Sulfate

The effect of potassium chloride and ammonium sulfate concentrations on the detection of low abundance target nucleic acids was also tested. As shown in this illustrative example, less than 10 copies of a target nucleic acid that differed from abundant nucleic acids by a single base change were detected by the primer and probe assay design illustrated in FIG. 1 and/or described in more detail below, and an amplification mixture containing optimized concentrations of both KCl and (NH₄)₂SO₄. Discrimination of low abundance target nucleic acids in the presence of highly similar and abundant nucleic acids is particularly difficult when the single base change is in the same structure family (purine to purine or pyrimidine to pyrimidine). It was demonstrated in these examples that potassium chloride improves amplification and ammonium sulfate improves discrimination of the low abundance target nucleic acid in the presence of the highly similar and abundant nucleic acids. The concentrations of both potassium chloride and ammonium sulfate were optimized for amplification of the low abundance target nucleic acid and suppression of amplification of the abundant target. As described generally above, the third oligonucleotide (e.g., TaqMan probe) of these assays were designed to bind a nucleotide spanning a target variant nucleotide of a target nucleic acid (e.g., an allelic variant). In addition to the third oligonucleotide (e.g., TaqMan probe), either the forward or the reverse primer was designed to include at its 3′ end a nucleotide complementary to the target variant nucleotide on the target nucleic acid. As discussed above, the primer including at its 3′ end a nucleotide complementary to the target variant is the first oligonucleotide (e.g., target sequence-specific primer (TSP)). The other primer (i.e., the second oligonucleotide), which has binding specificity for the target nucleic acid does not include at its 3′ end a nucleotide complementary to a target variant nucleotide as described herein, and can be referred to as the locus-specific primer (LSP).

### A. The Effect of Potassium Chloride and Ammonium Sulfate on Amplification

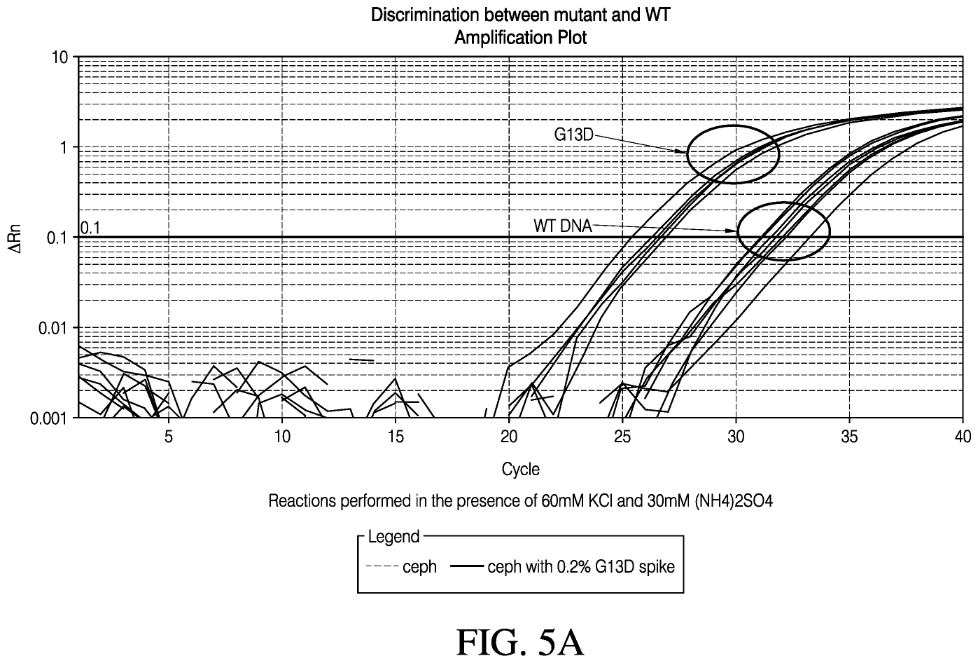

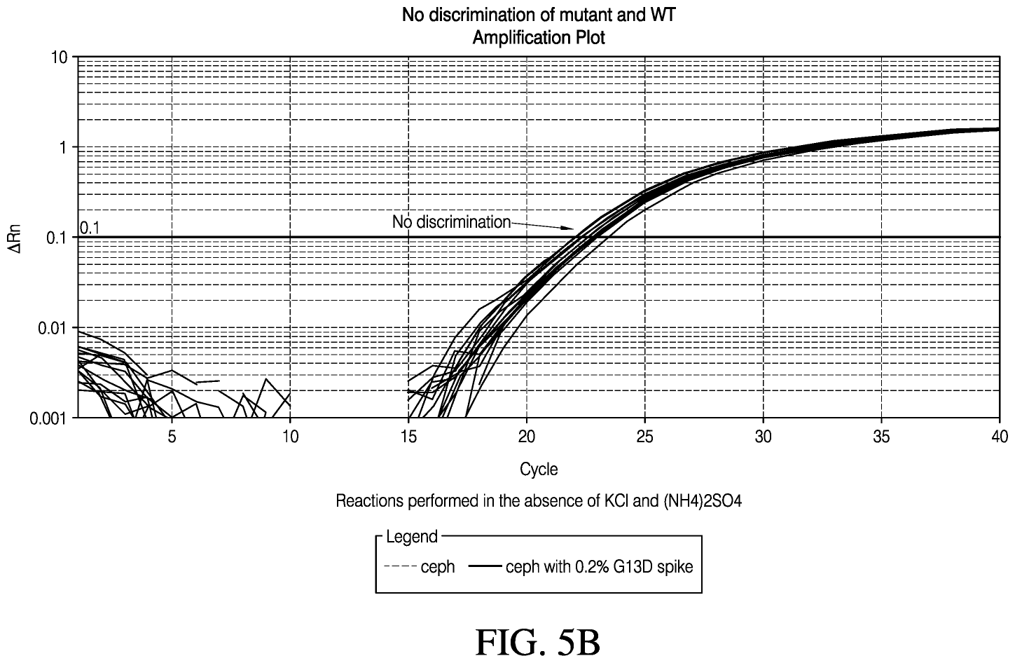

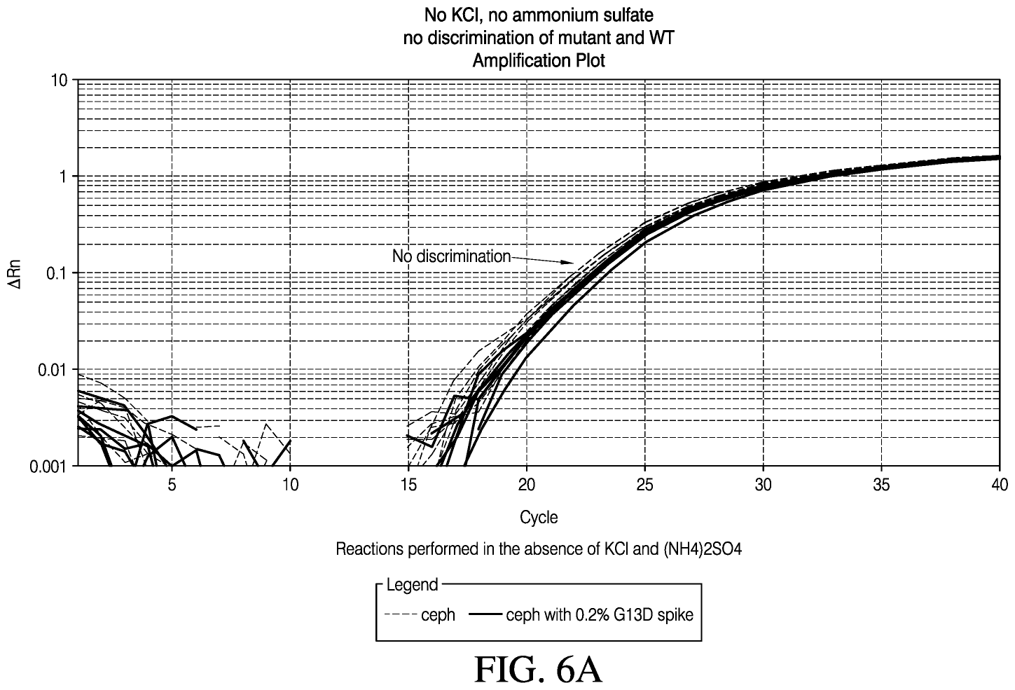

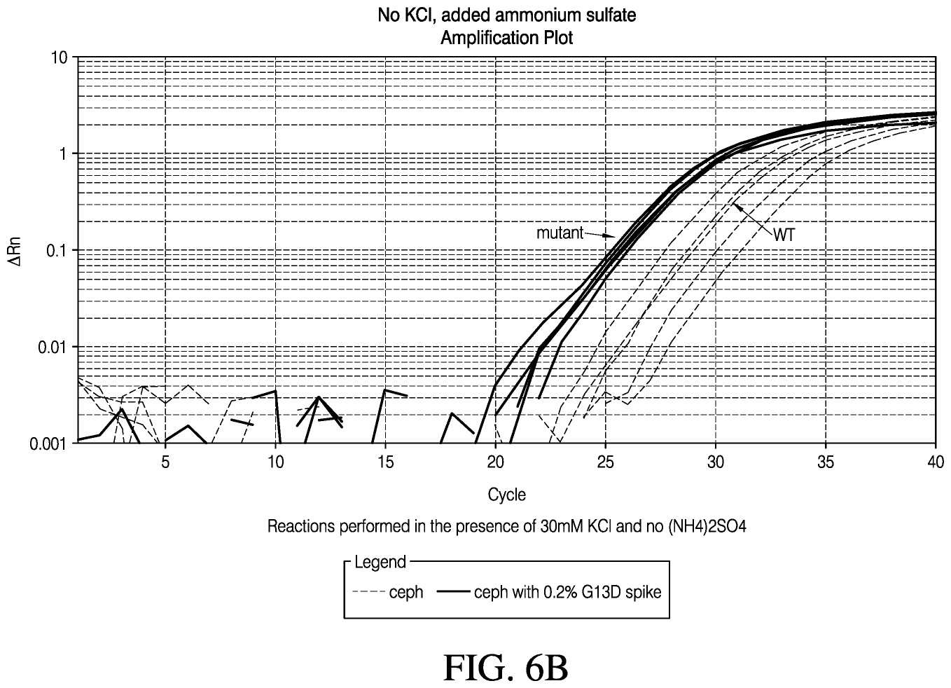

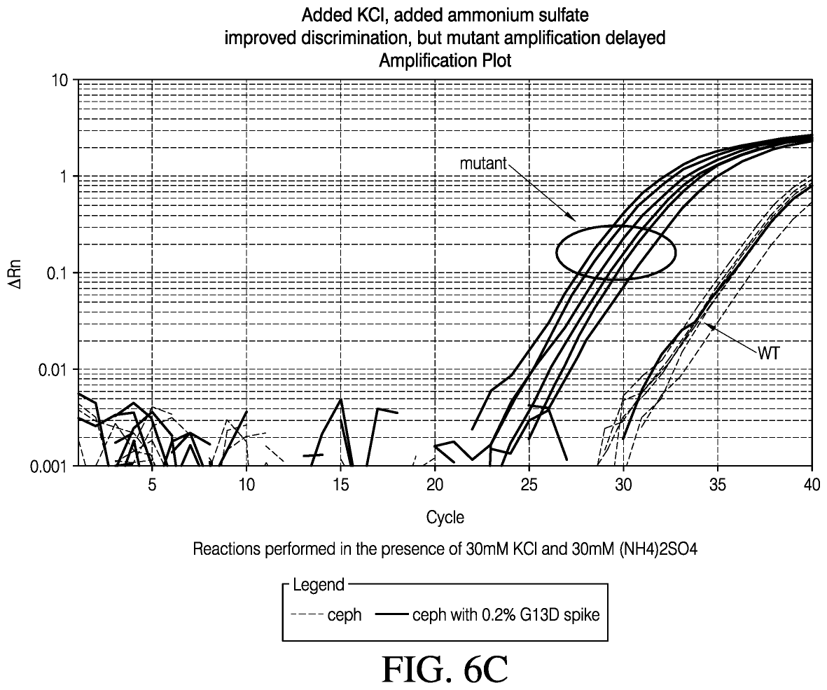

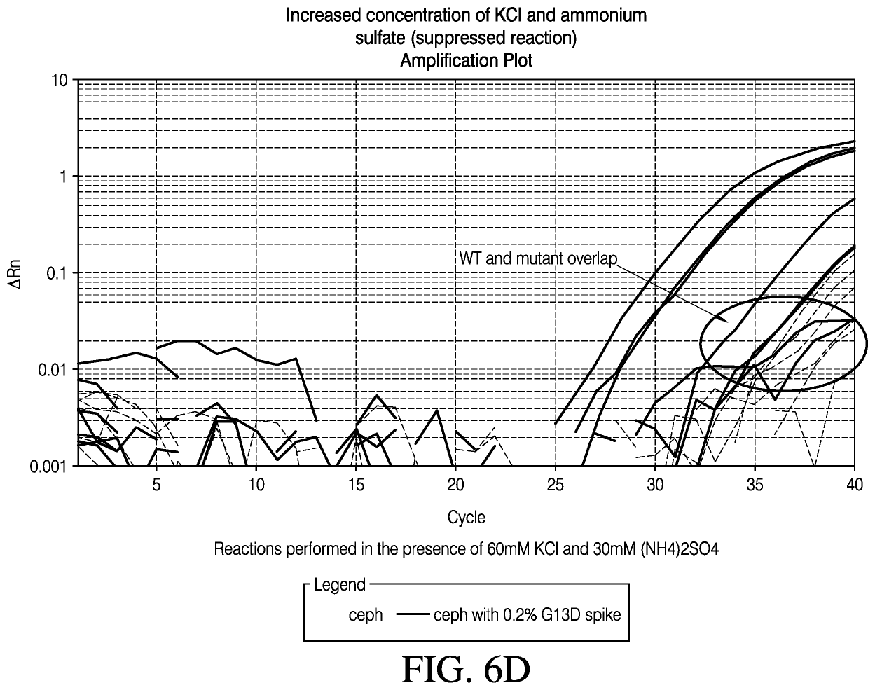

In these experiments, 20 uL reaction mixtures were made containing 0.3 μM forward [or reverse] primer (e.g., a first oligonucleotide (e.g., target sequence-specific primer (TSP)), 0.3 μM reverse [or forward] primer (e.g., a second oligonucleotide (e.g., locus-specific primer (LSP)), 0.25 μM detection probe (e.g., a third oligonucleotide (e.g., target site-specific probe)), an amplification reagent with 1 mM dNTPs, 2.55 mM MgCl₂, 39 mM Tris pH 8, 0.085U/uL Platinum Taq, and 0.1 mg/mL BSA; zero, 30 mM, or 60 mM KCl (data shown for zero and 60 mM only); and zero, 15 mM, or 30 mM (NH₄)₂SO₄ (data shown for zero and 30 mM only); and 10 ng of wild-type DNA; with or without 0.2% (4 copies) of the allele variant DNA spiked-in, as indicated in FIGS. 5 and 6. qPCR was performed on a QuantStudio 7 with the following cycles: 1 cycle of 95° C. for 3 min; 19 cycles of 95° C. for 3 sec/64° C. for 20 sec (enrichment phase); and 40 cycles of 95° C. for 3 sec/60° C. for 20 sec (amplification and detection phase). These experiments and the resultant data are further described below.

It was observed that qPCR reactions including up to 60 mM KCl and up to 30 mM (NH₄)₂SO₄, as well as the primer and probe assay design of FIG. 1 and further described above, discriminated samples containing only wild-type DNA versus wild-type DNA spiked with 0.2% of the allelic variant DNA, as shown by the lower Ct values (FIG. 5A; reactions performed in the presence of 60 mM KCl and 30 mM (NH₄)₂SO₄). In contrast, qPCR reactions that included neither salt had similar Ct values for reactions containing wild-type DNA and wild-type DNA spiked with 0.2% of the allelic variant DNA (FIG. 5B; reactions performed in the absence of KCl and (NH₄)₂SO₄). Thus, inclusion of an effective amount of KCl and (NH₄)₂SO₄ in a qPCR master mix, along with the primer and probe assay design provided in FIG. 1 (e.g., exemplified here as described above), provided an effective method for detecting as few as 0.2% (e.g., 4 copies) of a target nucleic acid in a sample comprising an abundant (e.g., wild-type) nucleic acid differing from the target nucleic acid by only a single nucleotide (i.e., the target variant nucleotide).

Additional experiments were carried out to determine a range of concentrations that would provide for differentiation of low copy number target and wild-type nucleic acids. 20 uL reaction mixtures containing 10 ng wild-type (CEPH) DNA (Thermo Fisher Scientific Cat. No. 403062); 300 nM each primer (forward and reverse; first and second oligonucleotides; TSP and LSP); 250 nM probe (third oligonucleotide); 1 mm dNTPs; 0.085U/uL Platinum Taq; 2.55 mM MgCl₂; 45 nM ROX passive reference; 39 mM Tris, pH8; and 7% glycerol were prepared. Potassium chloride and ammonium sulfate were titrated into the reaction mixtures as shown below: FIG. 6A: no KCl or ammonium sulfate; FIG. 6B: 30 mM ammonium sulfate, no potassium chloride; FIG. 6C: 30 mM KCl and 30 mM ammonium sulfate; and, FIG. 6D: 60 mM KCl and 30 mM ammonium sulfate. The reactions with G13D mutant spike contained 20 pg KRAS G13D Reference Standard DNA (Horizon Discovery Ltd.; Cat. No. HD290). The reactions were amplified on a QuantStudio7 instrument using the following thermal protocol: 95° C. (3 min); 95° C. (3 sec),/64° C. (20 sec) for 19 cycles (enrichment phase); then 95° C. (3 sec)/60° C. (20 sec) for 40 cycles (amplification and detection phase). As shown in FIG. 6C, the conditions used in the exemplary reaction (30 mM KCl and 30 mM ammonium sulfate) exhibited clear differentiation between target and wild-type nucleic acids while exclusion of both KCl and ammonium sulfate (FIG. 6A) did not. Thus, in these experiments, an “effective amount” (the amount providing for differentiation between target and wild-type nucleic acids) of KCl and ammonium sulfate was found to be up to about 60 mM and up to about 30 mM, respectively.

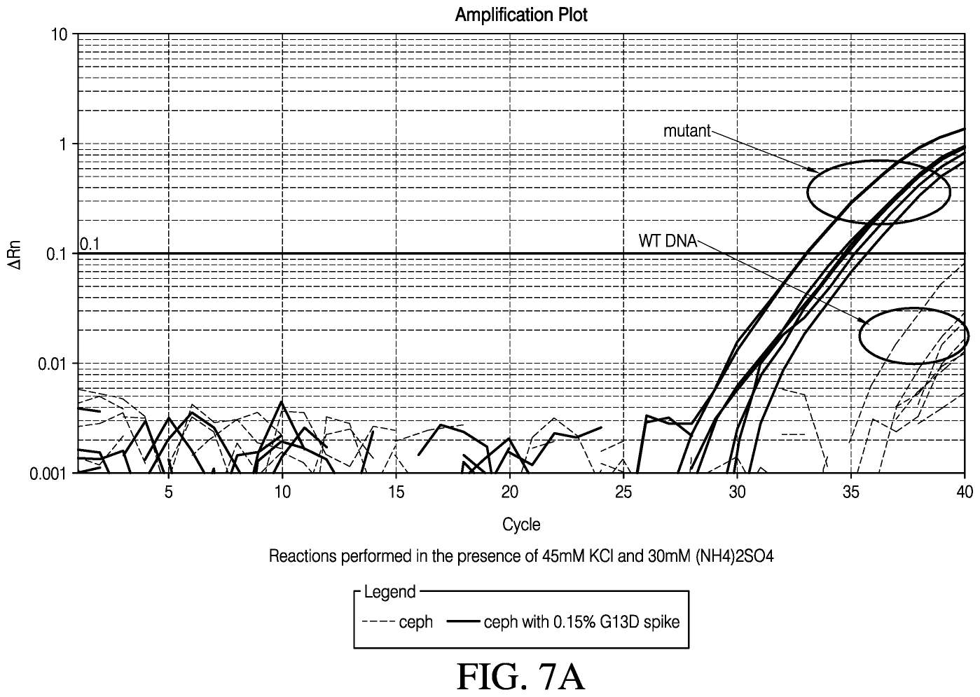

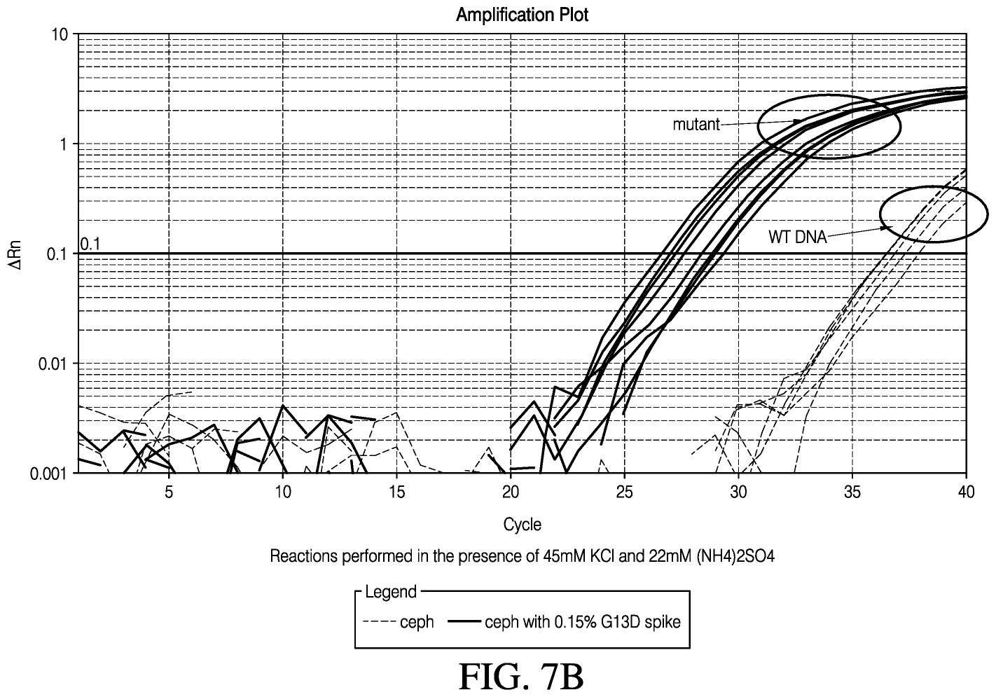

The effect of the two salts was further explored in experiments using KCl and ammonium sulfate concentrations between the initial high (60 mM KCl, 30 mM ammonium sulfate) and the initial low (30 mM KCl, 15 mM ammonium sulfate) concentrations were also tested. As FIG. 6C demonstrates, the effect of the two salts when used at the higher concentrations, provides discrimination, but also shows the Cq is delayed. Intermediate concentrations were therefore tested to determine if any might provide acceptable discrimination and lower Cq values for the less abundant target. In FIG. 7A, the concentrations of the two salts were 45 mM (KCl) and 30 mM (ammonium sulfate) while in FIG. 7B the concentration of ammonium sulfate was decreased to 22 mM (keeping KCl at 45 mM). In reactions performed in the presence of 45 mM KCl and 22 mM (NH₄)₂SO₄ (FIG. 7B), the Cq of the mutant was decreased while maintaining clear separation of the mutant from the wild-type polynucleotides. The average Cq of the wells with the mutant spike was decreased from 35.1 (FIG. 7A, for reactions using 30 mM ammonium sulfate) to 28.0 (FIG. 7B, for reactions using 22 mM ammonium sulfate), while maintaining separation from the wells containing wild-type alone. Based on these experiments, the use of 45 mM KCl and 22 mM ammonium sulfate was selected as optimal for further studies using similar conditions.

### Example 3

### Effect of Target Variant Nucleotide Distance from 3′ End of Probe

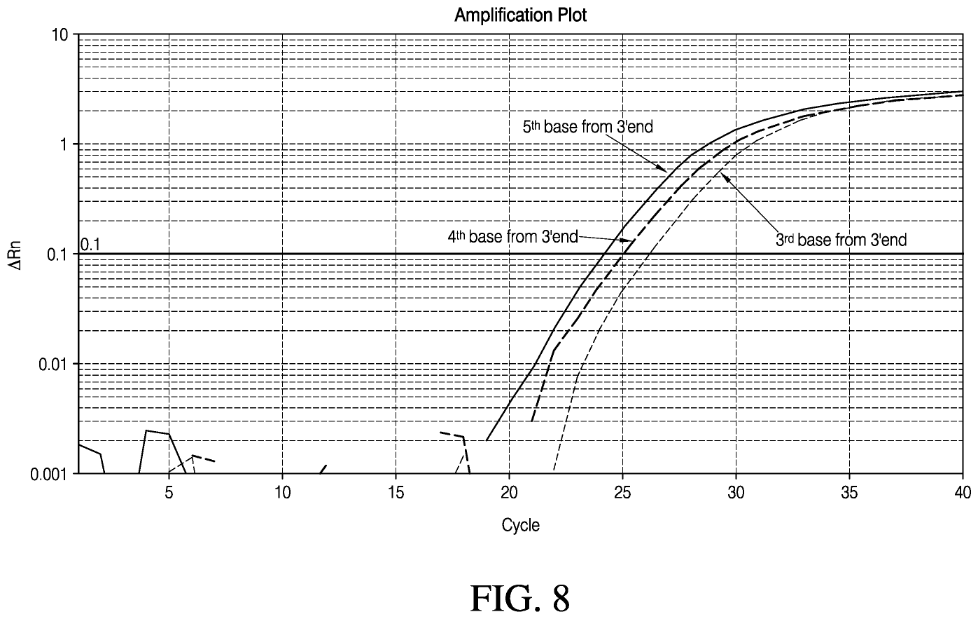

The effect of the distance of the target variable nucleotide from either end of the first oligonucleotide (e.g., target sequence-specific primer (TSP)), and with different numbers of bases of overhang that do not bind the target nucleic acid, was assessed. Experiments were carried out in 20 uL reactions containing) 1 mM dNTPs, 45 mM KCl, 22 mM ammonium sulfate, 0.085U/uL Platinum Taq, 2.55 mM MgCl₂, 45 nM ROX passive reference, 39 mM Tris pH 8 and 7% glycerol, 300 nM of each primer (TSP and LSP), 250 nM of one of Probes 1, 2 or 3; 10 ng wild-type (CEPH) DNA (Thermo Fisher Scientific Cat. No. 403962); and 10 pg of EGFR L858R Reference Standard DNA (Horizon Discovery Ltd.; Cat. No. HD254). The data presented in FIG. 8 illustrates that the effective amplification and real-time detection was achieved with the target variant nucleotide located more than 2 bases from the end of the probe (i.e., at 3, 4, or 5 nucleotides from the 3′ end, Probes 1, 2, and 3, respectively). Increasing the distance of the target variant nucleotide to 5 bases from the 3′ end (Probe 3) compared to 3 bases from the 3′ end (Probe 1) resulted in a decreased number of cycles at which the change in fluorescence of the 6-FAM reporter dye on the probe divided by a passive reference dye present in the reaction exceeded a cutoff of 0.1.

### Example 4

### Titration of Mutant DNA into Wild-Type DNA

### A. Detection of Target Nucleic Acids Using Different Primer Concentrations

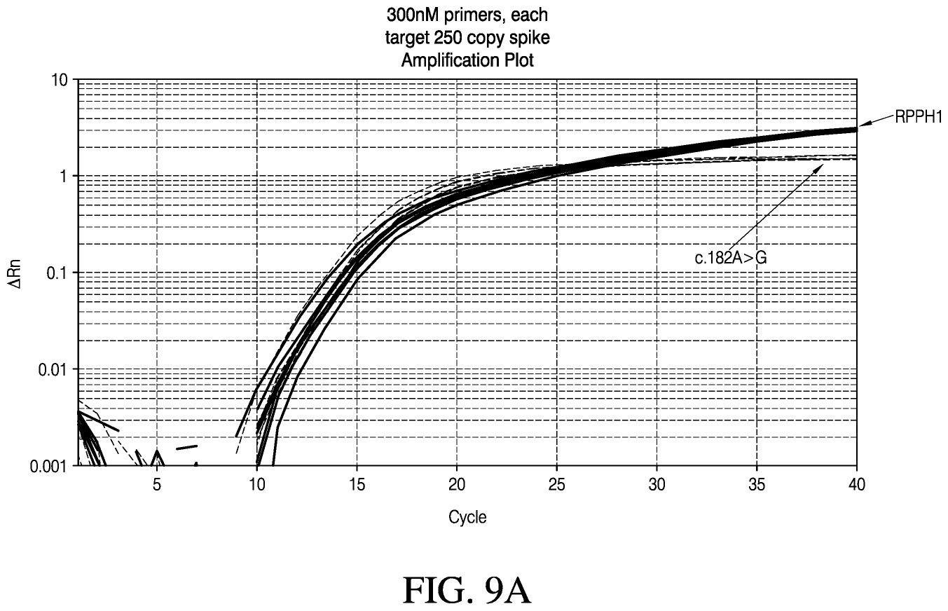

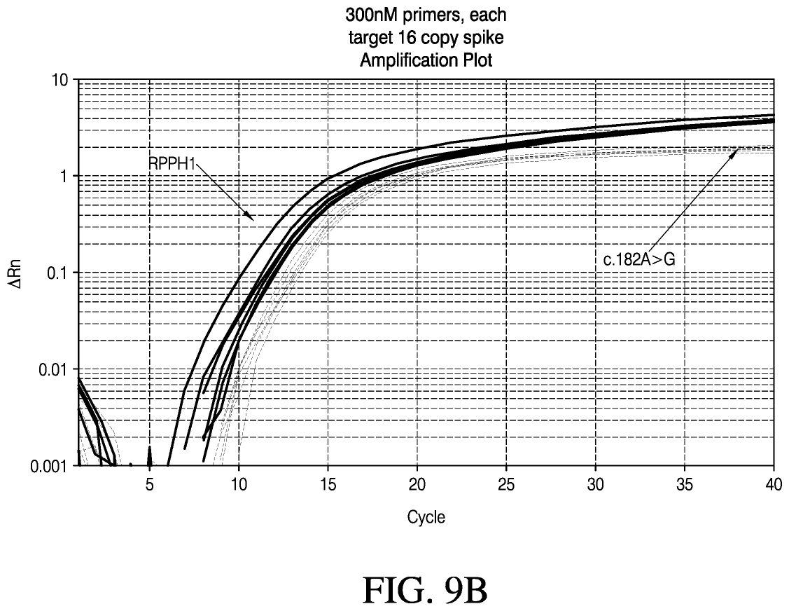

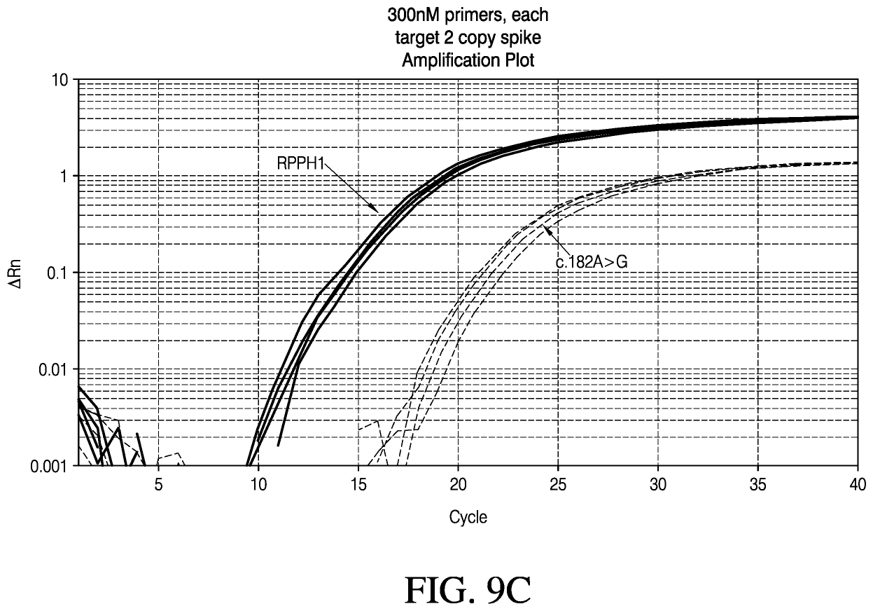

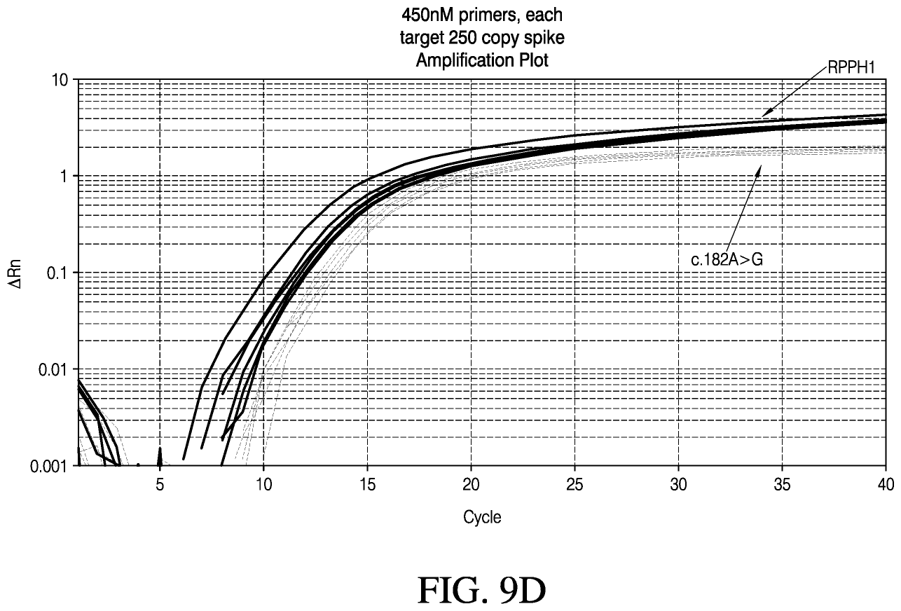

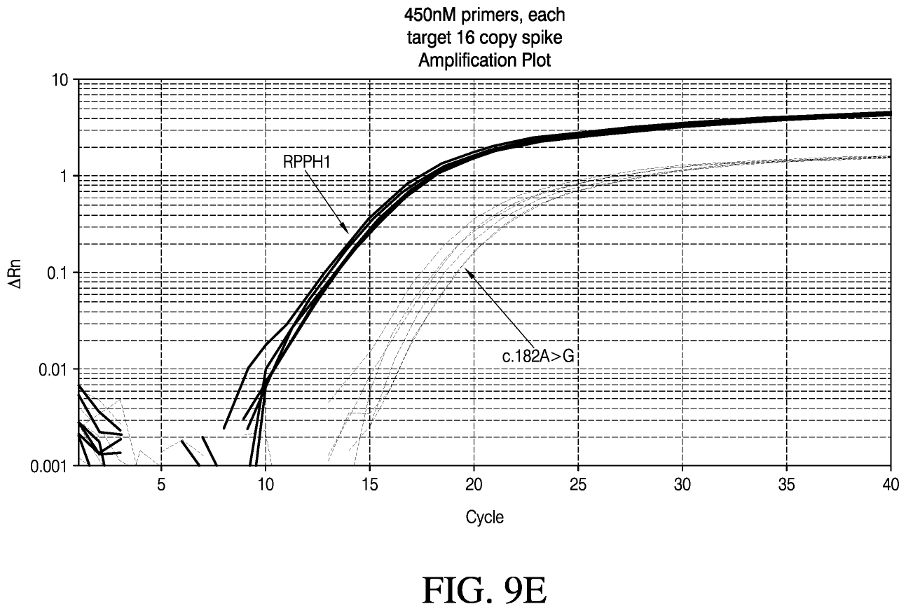

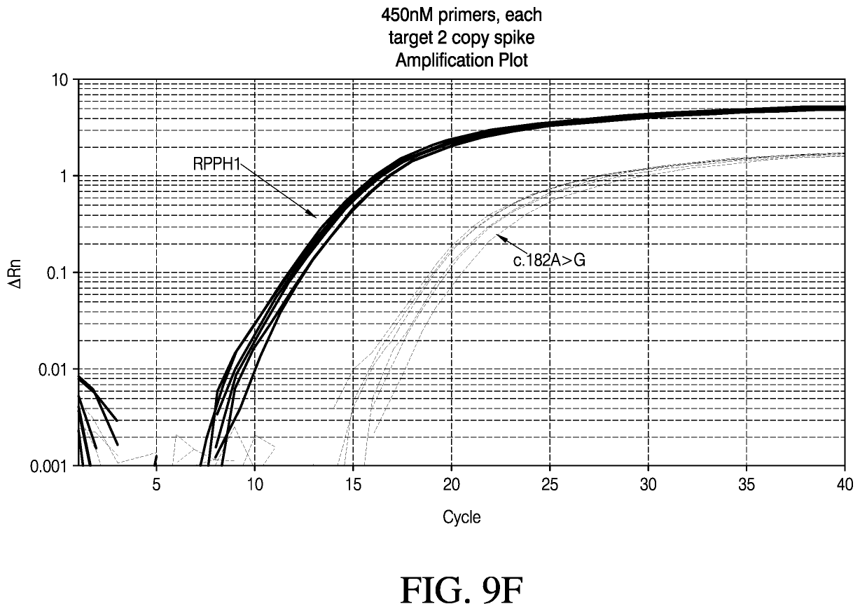

These experiments were performed in 20 uL reactions containing 10 ng wild-type (CEPH) DNA (Thermo Fisher Scientific Cat. No. 403062), and the indicated amount of mutant (Horizon Discover Ltd., Cat. Nos. HD574 (NRAS Q61R) spiked-in, 1 mM dNTPs, 45 mM KCl, 22 mM ammonium sulfate, 0.085U/uL Platinum Taq, 2.55 mM MgCl₂, 45 nM ROX passive reference, 39 mM Tris pH 8 and 7% glycerol. Reactions indicated with ‘300 nM primers’ contained 300 nM forward and reverse primers (TSP and LSP) for mutant and control targets (NRAS Q61R mutant target and RPPH1 control target), reactions indicated with ‘450 nM primers’ contained 450 nM forward and reverse primers (TSP and LSP) for both mutant and control targets. In all instances the FAM-labeled (mutant) and VIC-labeled (RPPH1) probe concentrations were 250 nM. The NRAS Q61R mutant target was added to the reactions at the indicated concentrations (e.g., 250 copies, 125 copies, 62.5 copies, 31 copies, 16 copies, 8 copies, 4 copies, and 2 copies). A 50fM solution of the mutant template (30000 copies/uL) was diluted to 3000 copies/uL, then to 250 copies/uL. From there 2-fold dilutions were performed down to 2 copies/uL. The reactions were amplified on a QuantStudio7 instrument using the following thermal protocol: 95° C. (3 min); 95° C. (3 sec)/64° C. (20 sec) for 19 cycles; then 95° C. (3 sec)/60° C. (20 sec) for 40 cycles. The data was exported in Excel format, the Cq values for replicate reactions averaged and the delta average Cq of targets in each condition determined and plotted (FIG. 10). The delta Cq was used a quantification method. As shown in FIGS. 9 and 10, the inclusion of either 300 nM or 450 nM primers combined with lower numbers of target nucleic acids (i.e., 2 or 16 copies) exhibited differentiation of the mutant target from the endogenous control target, RPPH1 (e.g., FIGS. 9C, 9E, and 9F).

### B. Detection Target Nucleic Acids at Different Copy Numbers

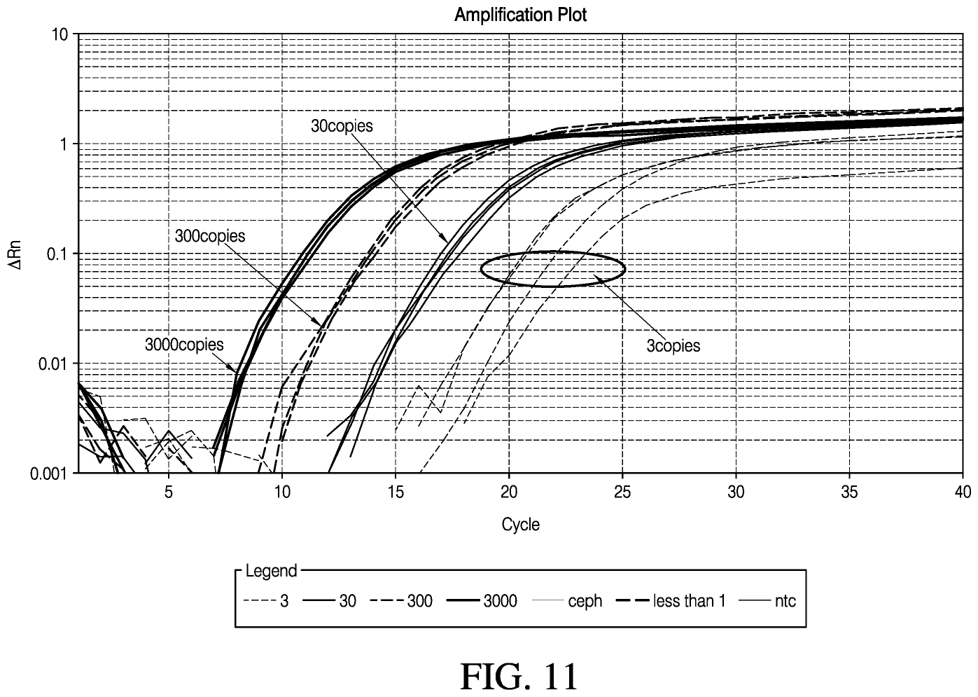

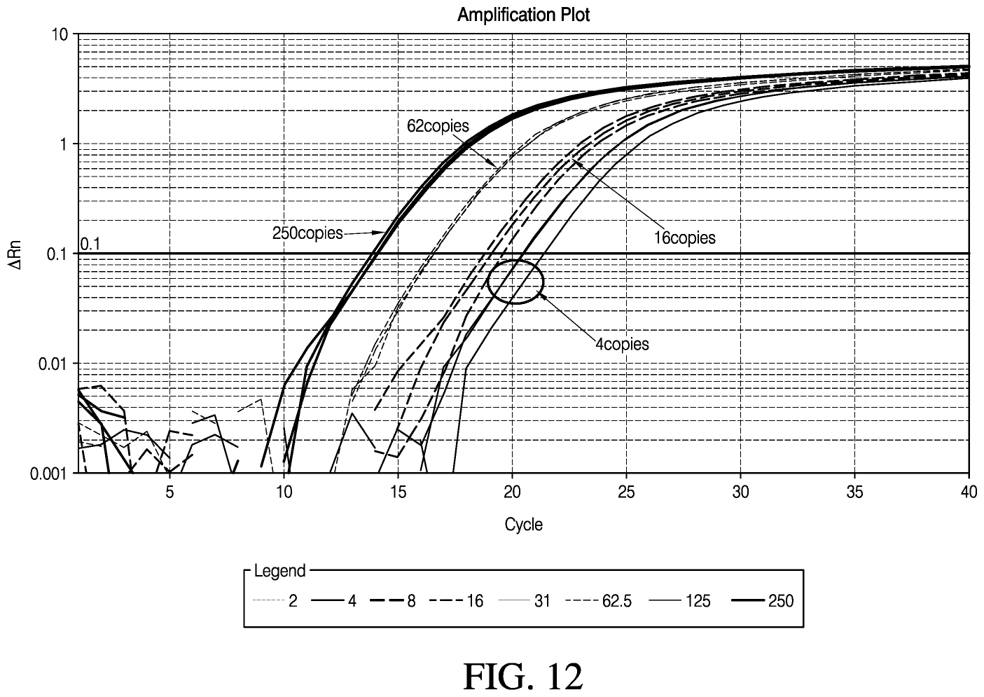

In these experiments, 20 uL reaction mixtures containing 300 nM each primer (TSP and LSP); 250 nM probe; 10 ng wild-type (CEPH) DNA (Thermo Fisher Scientific Cat. No. 403062); and the indicated amount and type of mutant (Horizon Discover Ltd., Cat. Nos. HD574 (NRAS Q61R) or HD351 (NRAS Q61K) spiked-in; 1 mM dNTPs; 45 mM KCl; 22 mM ammonium sulfate; 0.085U/uL Platinum Taq; 2.55 mM MgCl₂; 45 nM ROX passive reference; 39 mM Tris, pH 8; and 7% glycerol were prepared. The data was exported in Excel format, the Cq values for replicate reactions averaged and the delta average Cq of targets in each condition determined and plotted (data not shown). The delta Cq was used a quantification method. Thermal cycling conditions on Quant Studio 7 were: 95° C. (3 min); 19 cycles of 95° C. (3 sec)/64° C. (20 sec); 40 cycles of 95° C. (3 sec)/60° C. (20 sec). As shown in FIG. 11 (NRAS Q61R) and FIG. 12 (NRAS Q61K), as low as three and four copies of target nucleic acid could be detected using the system as described herein.

C. Reactions with Different Amount of Wild-Type DNA

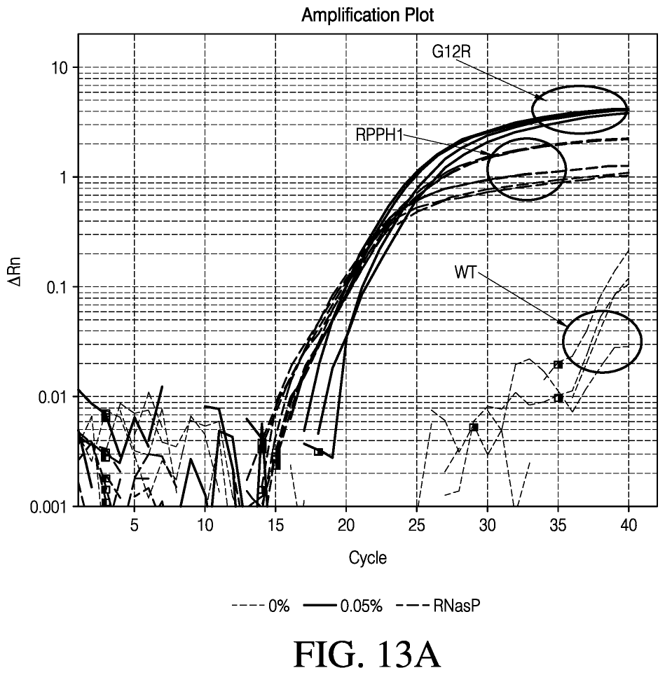

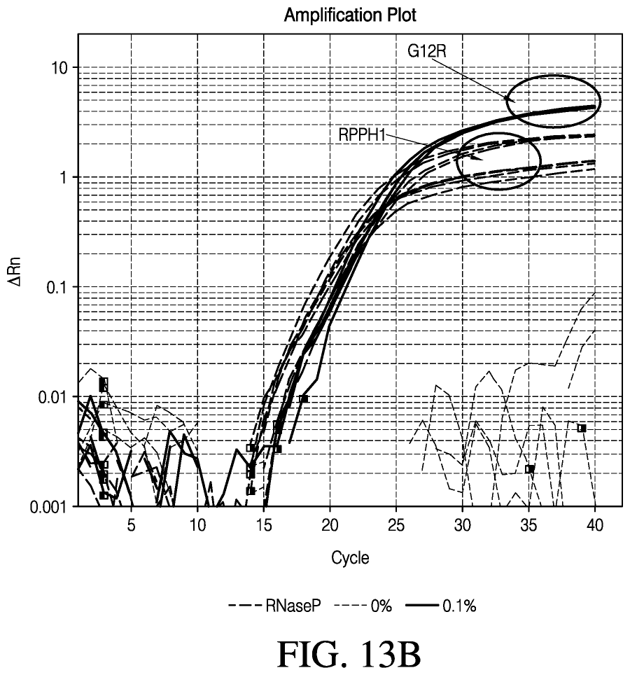

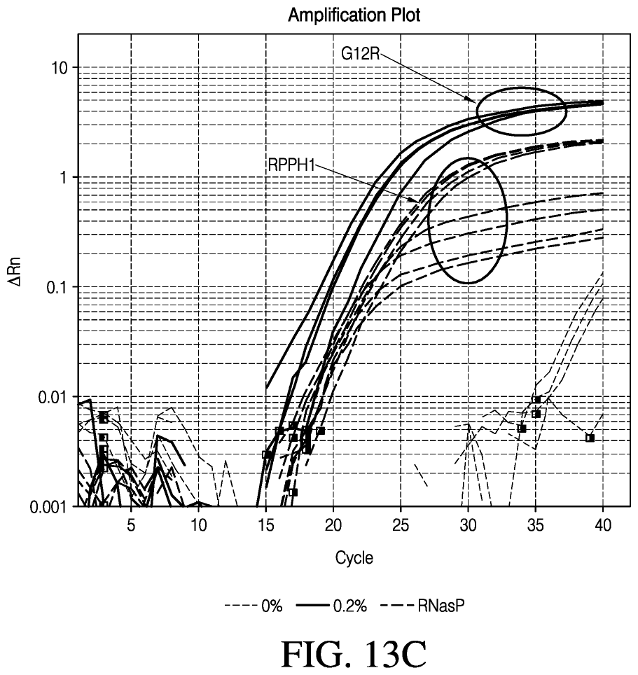

To carry out these experiments, reaction mixtures containing 300 nM of the KRAS forward and reverse (TSP and LSP) primers; 150 nM RPPH1 forward and reverse primers; 250 nM KRAS FAM probe; 150 nM RPPH1 VIC probe; 1 mM dNTPs; 45 mM KCl; 22 mM ammonium sulfate; 0.085U/uL Platinum Taq; 2.55 mM MgCl₂; 45 nM ROX passive reference; 39 mM Tris pH 8; and 7% glycerol were prepared. 10 pg KRAS G12R Reference Standard DNA (Horizon Discovery Ltd.; Cat. No. HD287); and, the indicated amount of wild-type (CEPH) DNA was added to each reaction. The reactions were run on a QuantStudio 5 using the following thermal protocol: 95° C. (2 min); 95° C. (1 sec)/64° C. (20 sec) for 19 cycles; then 95° C. (1 sec)/60° C. (20 sec) for 40 cycles. As shown in FIGS. 13A-C, mutant (e.g., lower abundant) target nucleic acids could be detected in a background of 5, 10, and 20 ng wild-type (e.g., higher abundant) DNA.

### Example 5

### Target Site at the 3′ End of a Reverse Primer

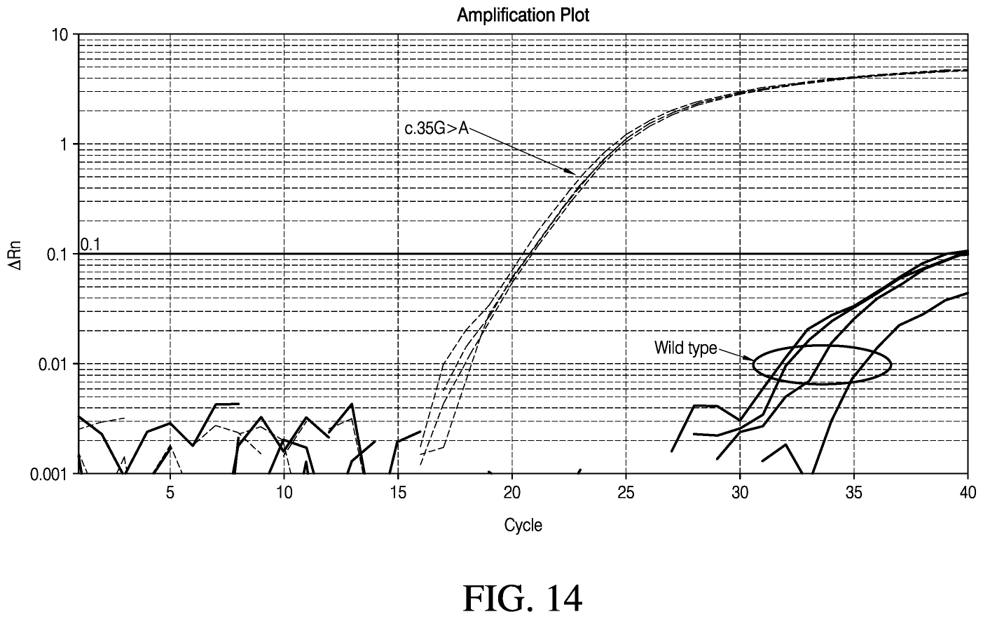

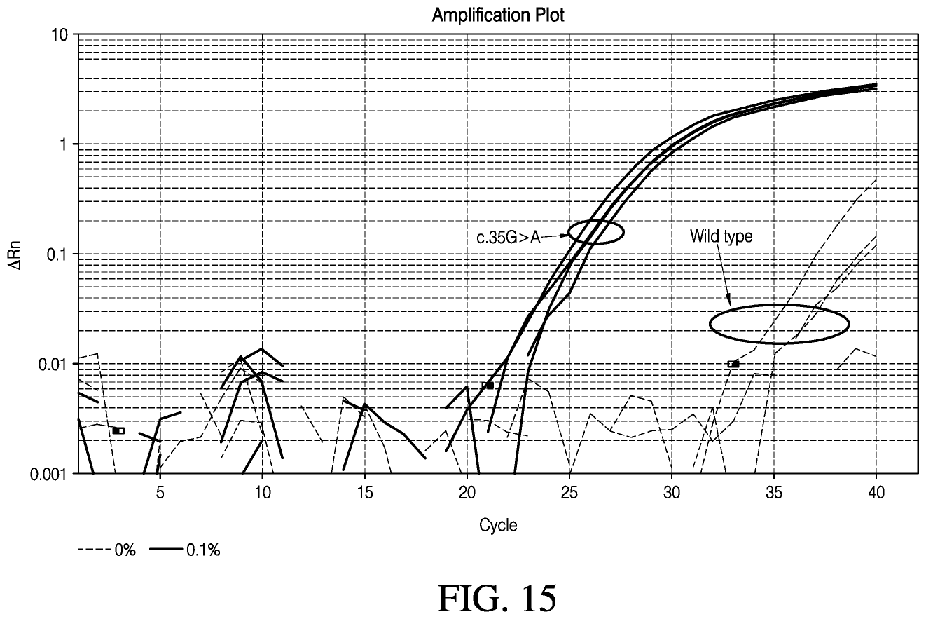

To carry out these experiments, 20 uL reaction mixtures containing 10 ng of CEPH DNA (Thermo Fisher Scientific Cat. No. 403062); 300 nM each primer (TSP and LSP); 250 nM probe; a mutant spike of 10 pg of KRAS G12D or G12S Reference Standard DNA (Horizon Discovery Ltd.; Cat. No. HD272 or HD288, respectively) were utilized as indicated; 1 mM dNTPs; 45 mM KCl; 22 mM ammonium sulfate; 0.085U/uL Platinum Taq; 2.55 mM MgCl₂; 45 nM ROX passive reference; 39 mM Tris, pH 8; and 7% glycerol were prepared. Thermal cycling conditions were: 95° C. (3 min), 19 cycles of 95° C. (3 sec), 64° C. (20 sec); followed by 40 cycles of 95° C. (3 sec)/60° C. (20 sec). As shown in FIG. 14 (KRAS G12D) and FIG. 15 (KRAS G12S), the reverse primer can be the target specific primer (TSP) (e.g., can be used for hybridization to either strand of a double-stranded polynucleotide—target site can be located at the 3′ end of either a forward or a reverse TSP primer) and the system as described herein functions properly, demonstrating differentiation between a low abundant (e.g., mutant) target and a high abundant (e.g., wild-type) target.

### Example 6

### Additional Genes and Rare Variants

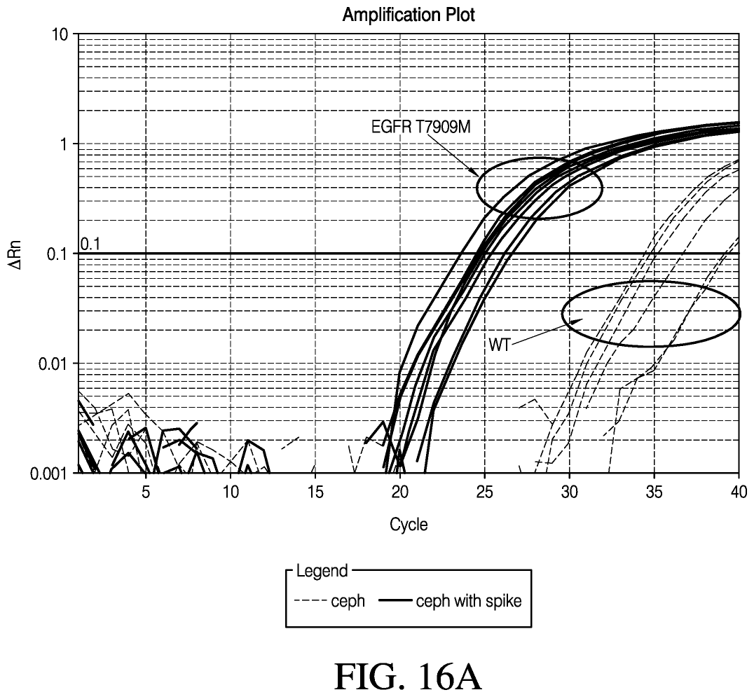

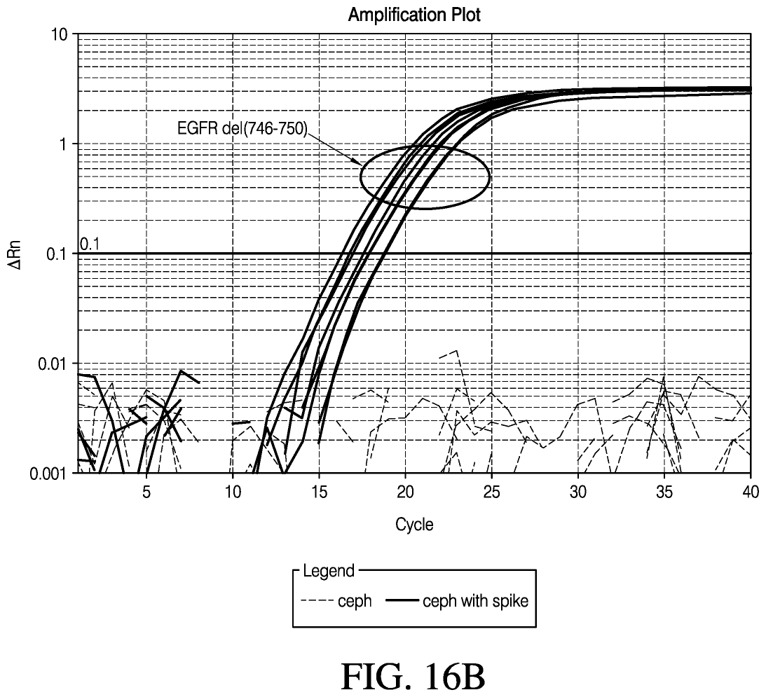

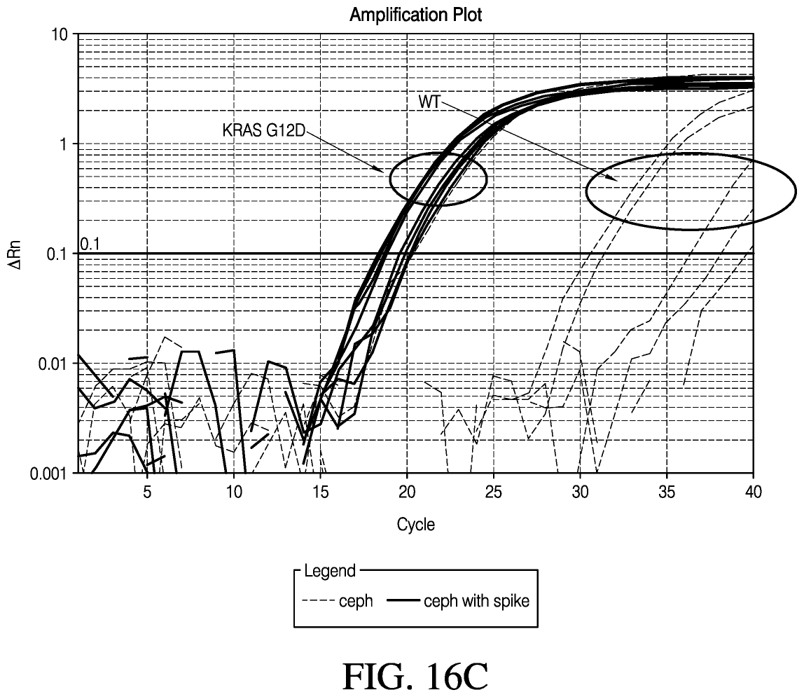

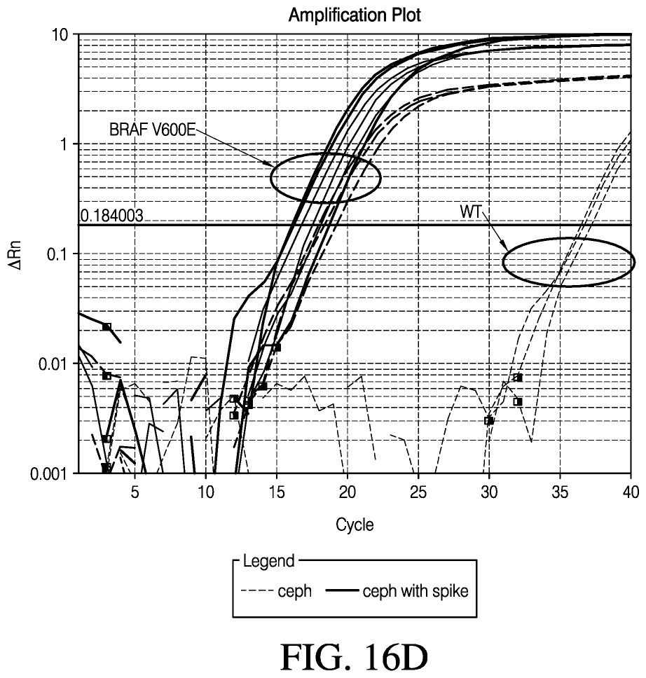

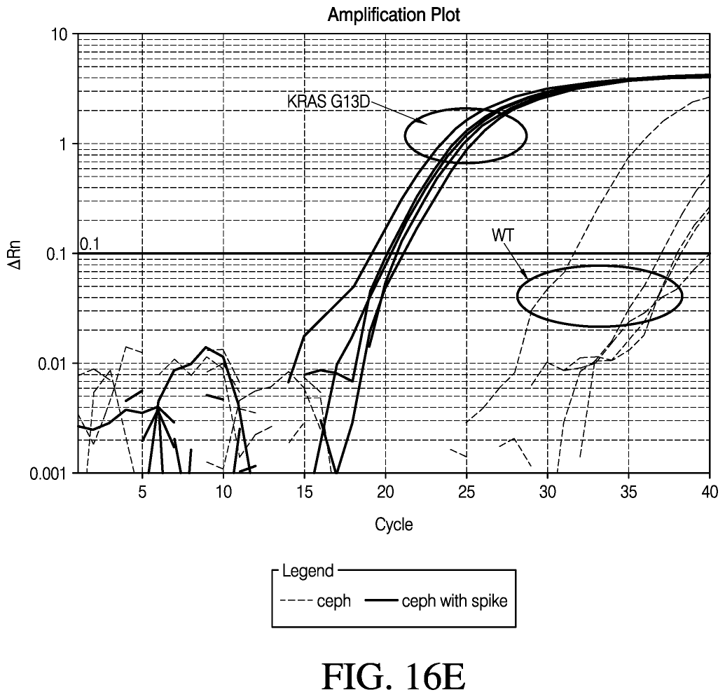

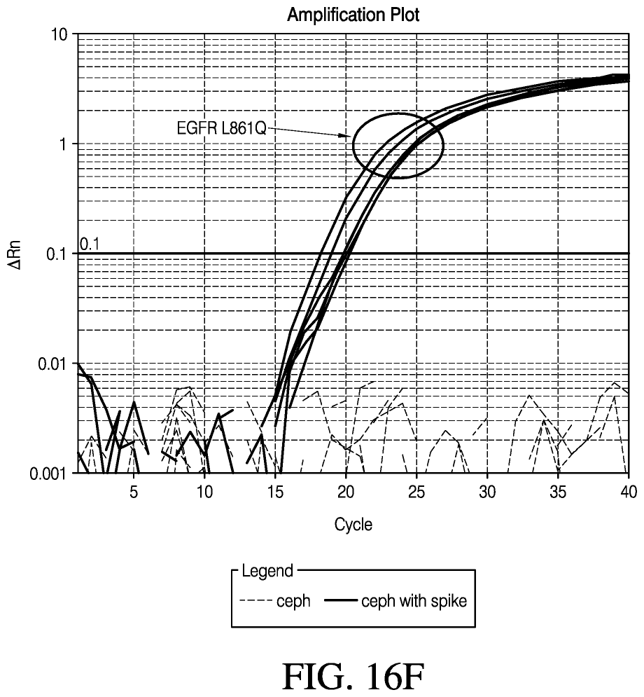

To carry out these experiments, 20 uL reaction mixtures containing 10 ng of wild-type (CEPH) DNA (Thermo Fisher Scientific Cat. No. 403062); 300 nM each of the first and second oligonucleotides (e.g., target sequence-specific primer (TSP) and locus-specific primer (LSP)); 250 nM probe (third oligonucleotide; target site-specific probe); a spike of 10 pg of the corresponding mutant DNA (e.g., mutant EGFR, BRAF, KRAS—as indicated in FIGS. 16A-16F; see Table 2) were utilized (Horizon Discovery Ltd.; Reference Standards Cat. Nos. as provided elsewhere herein) for the mutant samples; 1 mM dNTPs; 45 mM KCl; 22 mM ammonium sulfate; 0.085U/uL Platinum Taq; 2.55 mM MgCl₂; 45 nM ROX passive reference; 39 mM Tris, pH 8; and 7% glycerol were prepared. The data was exported in Excel format, the Cq values for replicate reactions averaged and the delta average Cq of targets in each condition determined and plotted (data not shown). The delta Cq was used a quantification method. The thermal cycling conditions used were: 95° C. (3 min), 19 cycles of 95° C. (3 sec)/64° C. (20 sec); followed by 40 cycles of 95° C. (3 sec)/60° C. (20 sec). As shown in FIGS. 16A-16F, each of the various target mutant nucleic acids tested, such as those listed in Table 2, were clearly differentiated from wild-type nucleic acids.

### Example 7

### Reactions with Probes of Different Length

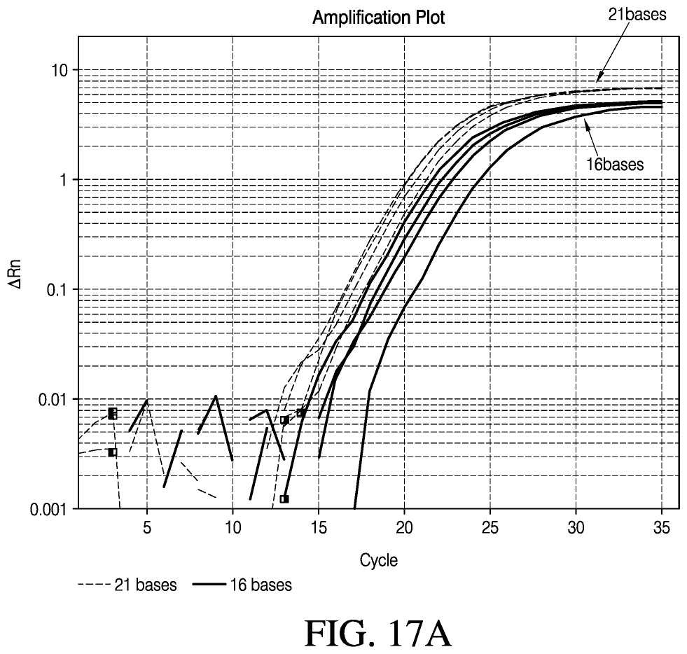

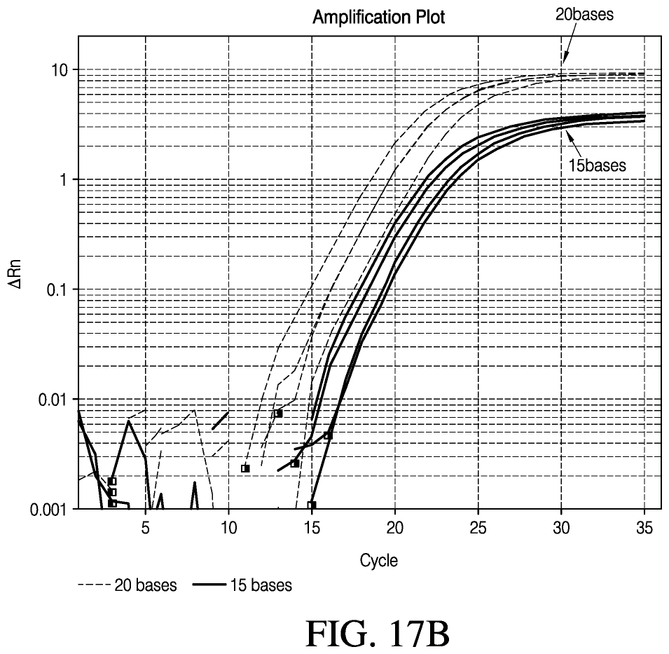

To carry out these experiments, 20 uL reaction mixtures containing 300 nM each primer; 250 nM of the appropriate probe; 10 ng wild-type (CEPH) DNA (Thermo Fisher Scientific Cat. No. 403062); a mutant spike of 20 pg of NRAS Q61L or Q61H Reference Standard DNA, as indicated (Horizon Discovery Lt.; Cat. No. HD412 or HD303, respectively); 1 mM dNTPs; 45 mM KCl; 22 mM ammonium sulfate; 0.085U/uL Platinum Taq; 2.55 mM MgCl₂; 45 nM ROX passive reference; 39 mM Tris, pH 8; and 7% glycerol were prepared. The reactions were run on a QuantStudio 5 using the following thermal protocol: 95° C. (2 min); 95° C. (1 sec)/64° C. (20 sec) for 19 cycles; then 95° C. (1 sec)/60° C. (20 sec) for 40 cycles. As shown in FIG. 17, varying the probe length by a difference of up to 5 nucleotides had little effect on the ability to detect low abundant targets in these experiments, although in some other instances probe length has been observed to lower the Cq value (data not shown). FIG. 17A demonstrates detection of NRAS Q61L (182A>T), using two different probes having a length of either 16 or 21 nucleotides. FIG. 17B demonstrates detection of NRAS Q61H (183A>T), using two different probes having a length of either 15 or 20 nucleotides.

### Example 8

### A Variety of Mutant Targets Tested

When reference materials for a target were not commercially available artificial double-stranded DNA templates were used. An artificial dsDNA fragment of 300 bp having the indicated mutations were ordered from Thermo Fisher Scientific (GeneArt Strings DNA Fragments, Cat. No. 815010DE). The dsDNA fragments were diluted to 50fM (30000 copies/uL) based on the Certificate of Analysis provided with each dsDNA product. Subsequent dilutions were made as needed for addition to the individual qPCR reactions. Each 20 uL reaction also contained 10 ng of wild-type (CEPH) DNA. In addition to the wild-type DNA (10 ng CEPH) and artificial templates, as indicated, each 20 uL reaction contained 300 nM each of the first and second oligonucleotides (e.g., target sequence-specific primer (TSP) and locus-specific primer (LSP) for either the indicated mutant targets or a RPPH1 control target; 250 nM probe for either the indicated mutant targets or a RPPH1 control target, which were differentially labeled; 1.3 mM dNTPs 45 mM KCl; 22 mM ammonium sulfate; 0.175U/uL Platinum Taq; 2.85 mM MgCl2; 45 nM ROX passive reference; 40 mM Tris, pH 8; and 7% glycerol were prepared. For duplex reactions comprising probes for both a mutant target and the control target, the data was exported in Excel format, the Cq values for replicate reactions averaged, and the delta average Cq of targets in each condition determined and plotted (data not shown). The delta Cq between the indicated mutant target and the RPPH1 target was used as a quantification method. The thermal cycling conditions used were: 95oC (3 min), 19 cycles of 95oC (3 sec)/64oC (20 sec); followed by 40 cycles of 95oC (3 sec)/60oC (20 sec).

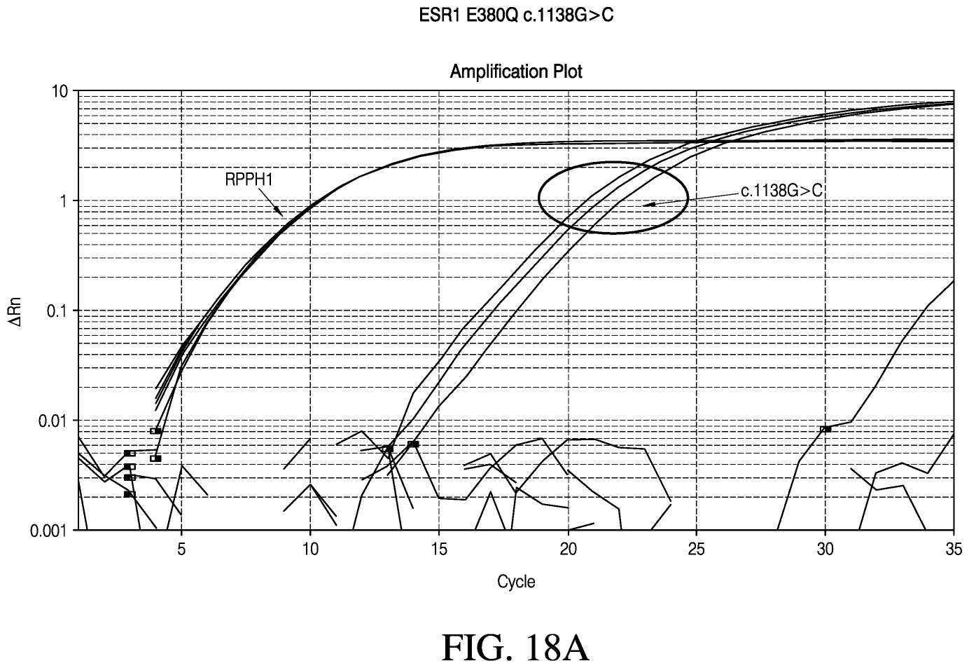

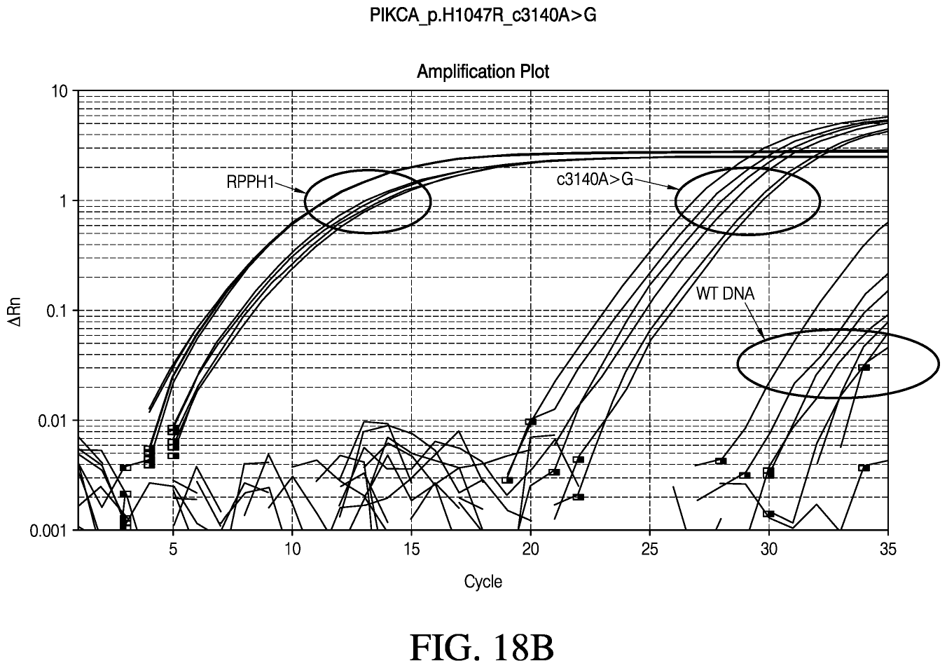

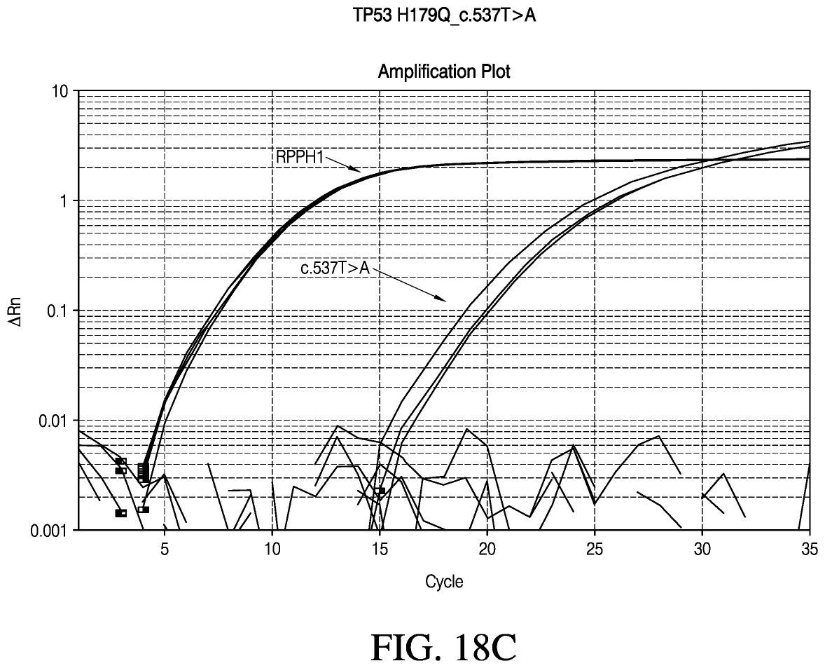

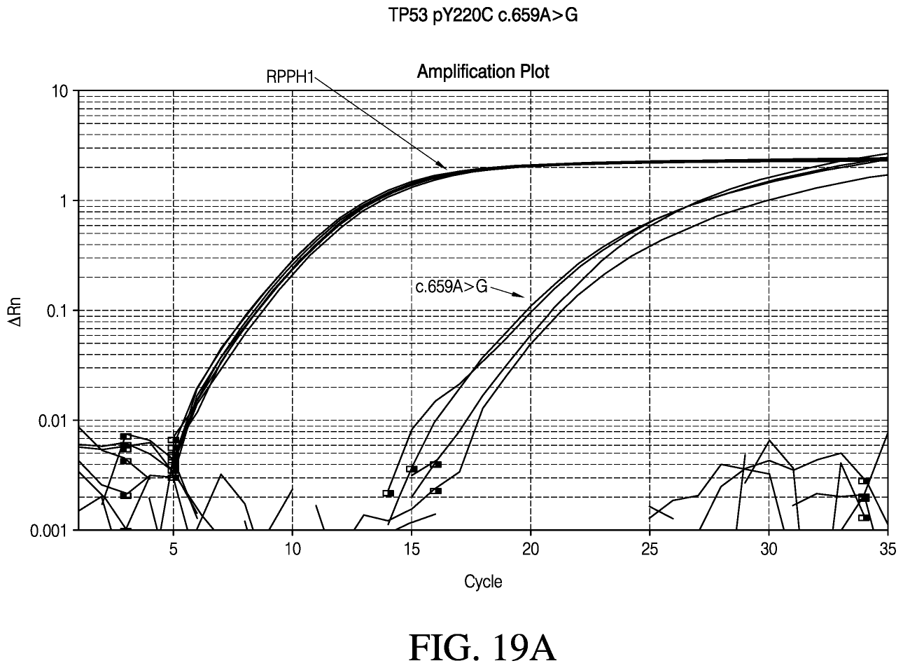

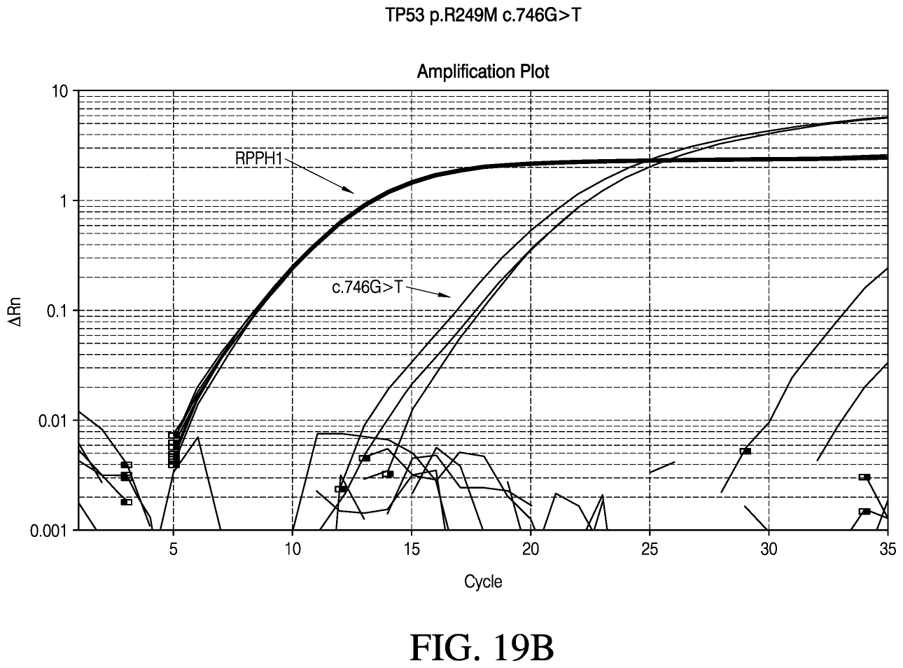

As shown in FIG. 18A through 18C and in FIGS. 19A and 19B, a number of artificial mutant targets, such as those listed in Table 2, were detected using the methods as described herein.

The foregoing examples illustrate various aspects of the invention and practice of the methods of the invention. The examples are not intended to provide an exhaustive description of the many different embodiments of the invention. Thus, although the forgoing invention has been described in some detail by way of illustration and example for purposes of clarity of understanding, those of ordinary skill in the art will realize readily that many changes and modifications can be made thereto without departing from the spirit or scope of the following clauses and appended claims.

Additional embodiments may be in accordance with following numbered clauses:

1. A mixture comprising:
a) a first oligonucleotide configured to hybridize to a first sequence in a first target polynucleotide strand, wherein the first sequence includes a target variant nucleotide, and wherein the first oligonucleotide further has a nucleotide residue at its 3′-end that is positioned to hybridize to the target variant nucleotide; and
b) a second oligonucleotide having a sequence configured to hybridize to a sequence complementary to a second sequence within the first target polynucleotide strand, wherein the second sequence of the first target polynucleotide strand is located 5’ upstream from the first sequence of the first target polynucleotide strand.
2. The mixture of clause 1, further comprising:
c) a third oligonucleotide having a sequence configured to hybridize to a sequence complementary to a third sequence within the first target polynucleotide strand, wherein the third sequence of the first target polynucleotide strand overlaps at least partially with the first sequence of the first target polynucleotide strand and the third sequence includes the target variant nucleotide.
3. A mixture comprising:
a) a first oligonucleotide configured to hybridize to a first sequence (A) present within a first target polynucleotide strand, wherein the first sequence includes a target variant nucleotide (“first variant nucleotide”), and wherein the first oligonucleotide further has a nucleotide residue at its 3′-end that is positioned to hybridize to the first variant nucleotide; and
b) a second oligonucleotide configured to hybridize to a second sequence (B), wherein the second sequence is complementary to a third sequence (C), the third sequence being present within the first target polynucleotide strand, wherein the third sequence (C) is located 5’ upstream from the first sequence (A) of the first target polynucleotide strand.
4. The mixture of clause 2, further comprising:
c) a third oligonucleotide configured to hybridize to a fourth sequence (D) complementary to a fifth sequence (E), the fifth sequence present in the first target polynucleotide strand, wherein the fifth sequence (E) overlaps at least partially with the first sequence (A) in the first target polynucleotide strand and includes the first target variant nucleotide.
5. The mixture of clause 2 or 4, wherein the target variant nucleotide in the third oligonucleotide is at least two nucleotides from a 3′ end or a 5′ end of the third oligonucleotide.
6. The mixture of any one of clauses 2, 4 and 5, wherein the third oligonucleotide comprises a detectable label.
7. The mixture of clause 6, wherein the detectable label is a fluorescent label.
8. The mixture of clause 6 or 7, wherein the detectable label is on a first terminal nucleotide.
9. The mixture of any one of clauses 6-8, wherein the third oligonucleotide further comprises a quenching moiety.
10. The mixture of clause 9, wherein the quenching moiety is on a second terminal nucleotide of the third oligonucleotide.
11. The mixture of clause 9 or 10, wherein the quenching moiety is capable of quenching a signal from the detectable label.
12. The mixture of clause 10, wherein the first terminal nucleotide is the 5′ terminal nucleotide.
13. The mixture of clause 10, wherein the second terminal nucleotide is the 3′ terminal nucleotide.
14. The mixture of clause 2 or 4, wherein the mixture comprises a single-stranded polynucleotide molecule including the first target polynucleotide strand.
15. The mixture of clause 4, wherein sequences A, E and C are located within a single-stranded polynucleotide molecule on the first target polynucleotide strand.
16. The mixture of clause 2 or 4, wherein the mixture comprises a double-stranded polynucleotide molecule including the first target polynucleotide strand and a first target complement polynucleotide strand, wherein the first target complement polynucleotide strand is substantially complementary to the first target polynucleotide strand.
17. The mixture of clause 4, wherein the mixture comprises a double-stranded polynucleotide molecule including the first target polynucleotide strand and a first target complement polynucleotide strand, wherein the first target complement polynucleotide strand is substantially complementary to the first target polynucleotide strand, and wherein sequences D and B are located within the double-stranded polynucleotide molecule on the target complement polynucleotide strand and sequences A, E, and C are located within the double-stranded polynucleotide molecule on the target polynucleotide strand.
18. The mixture of clause 2 or 4, wherein the mixture comprises a double-stranded polynucleotide molecule including the first target polynucleotide strand and a first target complement polynucleotide strand, wherein the first target complement polynucleotide strand is substantially complementary to the first target polynucleotide strand, and a double-stranded polynucleotide molecule including a variant polynucleotide strand and a variant complement polynucleotide strand, wherein the variant polynucleotide strand is substantially identical to the target polynucleotide strand and comprises a different nucleotide at the target variant nucleotide than the target polynucleotide strand, and wherein the variant complement polynucleotide strand is substantially complementary to the variant polynucleotide strand.
19. The mixture of clause 2 or 4, wherein the third oligonucleotide comprises 3 to 6 contiguous nucleotides of the first sequence.
20. The mixture of clause 2 or 4, wherein the third oligonucleotide further comprises a sequence of nucleotides of the first target polynucleotide strand that does not overlap with a sequence of nucleotides of the first sequence.
21. The mixture of clause 2 or 4, wherein the mixture does not comprise a fourth oligonucleotide.
22. The mixture of clause 2 or 4, wherein the mixture does not comprise a fourth oligonucleotide comprising a detectable label.
23. The mixture of clause 2 or 4, wherein the mixture does not comprise a fourth oligonucleotide that binds to the first target polynucleotide strand.
24. The mixture of clause 2 or 4, wherein the mixture does not comprise a fourth oligonucleotide having a detectable label and a sequence that binds to the first target polynucleotide strand.
25. The mixture of clause 2 or 4, wherein the third oligonucleotide is the only oligonucleotide in the mixture having a detectable label and a sequence configured to hybridize to a sequence of the target polynucleotide strand.
26. The mixture of clause 2 or 4, wherein the mixture further comprises between 30 mM and 80 mM potassium chloride.
27. The mixture of clause 26, wherein the concentration of potassium chloride is at least 40 mM.
28. The mixture of clause 26, wherein the concentration of potassium chloride is less than 70 mM.
29. The mixture of clause 26, wherein the concentration of potassium chloride is at least 40 mM and less than 70 mM.
30. The mixture of clause 2 or 4, wherein the mixture further comprises between 10 mM and 40 mM ammonium sulfate.
31. The mixture of clause 30, wherein the concentration of ammonium sulfate is at least 20 mM.
32. The mixture of clause 30, wherein the concentration of ammonium sulfate is less than 35 mM.
33. The mixture of clause 30, wherein the concentration of ammonium sulfate is at least 20 mM and less than 35 mM.
34. The mixture of clause 30, wherein the concentration of ammonium sulfate is 20 to 25 mM.
35. The mixture of clause 2 or 4, wherein the mixture further comprises:
a) a concentration of potassium chloride between 30 mM and 80 mM; and,
b) a concentration of ammonium sulfate between 10 mM and 40 mM.
36. The mixture of clause 35, wherein the concentration of potassium chloride is 40 mM to 70 mM and the concentration of ammonium sulfate is 20 mM to 35 mM.
37. The mixture of clause 35, wherein the concentration of potassium chloride is 40 mM to 48 mM and the concentration of ammonium sulfate is 20 to 24 mM.
38. The mixture of clause 35, wherein the concentration of potassium chloride is 45 mM and the concentration of ammonium sulfate is 22 mM.
39. The mixture of clause 6, wherein the detectable label is selected from the group consisting of a DNA-binding dye, reporter dye, fluorescent probe, 6-carboxyfluorescein (FAM™), tetrachlorofluorescin (TET™), 6-Carboxy-4′,5′-Dichloro-2′,7′-Dimethoxyfluorescein, Succinimidyl Ester (JOE™), VIC™, a sulfonate derivative of a fluorescein dye with SO₃ instead of the carboxylate group, a phosphoramidite form of fluorescein, a phosphoramidite form of CY5, a non-FRET label, a ferrocene reagent, ABY™ NED™ JUN™ Fluor®488, AlexaFluor®532, AlexaFluor®546, AlexaFluor®594, AlexaFluor®647, AlexaFluor®660, TYE™ 563 TYE™665 TYE™705, and combinations thereof.
40. The mixture of any one of clauses 9-11, wherein the quenching moiety is selected from the group consisting of tetramethylrhodamine (TAMRA), a non-fluorescent quencher (NFQ), Black Hole Quencher, Iowa Black Quencher, QSY quencher, QSY7 quencher, QSY21 quencher, Dabsyl and/or Dabsyl sulfonate/carboxylate quenchers.
41. The mixture of clause 2 or 4, wherein the Tₘ of the third oligonucleotide is at least 5° C. and no more than 25° C. higher than the Tₘ of the first oligonucleotide.
42. The mixture of clause 41, wherein the Tₘ of the third oligonucleotide is at least 8° C. and no more than 12° C. higher than the Tₘ of the first oligonucleotide.
43. The mixture of clause 2 or 4, wherein the Tₘ of the first oligonucleotide is within 5° C. of the Tₘ of the second oligonucleotide.
44. The mixture of any one of clauses 42 or 43, wherein the Tₘ of the first oligonucleotide is 45 to 60° C. and the Tₘ of the second oligonucleotide is 45 to 60° C.
45. The mixture of any one of clauses 1-4, wherein the first and second oligonucleotides are extendable.
46. The mixture of any one of clauses 1-4, wherein the first and second oligonucleotides are primers.
47. The mixture of clause 2 or 4, wherein the third oligonucleotide is non-extendable.
48. The mixture of clause 2 or 4, wherein the third oligonucleotide is a probe.
49. The mixture of clause 2 or 4, wherein the first, second, and/or third oligonucleotides comprise between 10-40 nucleotides.
50. The mixture of clause 2 or 4, wherein the third oligonucleotide comprises a blocking moiety.
51. The mixture of clause 50, wherein the blocking moiety is a minor groove binder (MGB) moiety.
52. The mixture of clause 51, wherein the MGB moiety is located at the 3′-end and/or 3′ portion of the third oligonucleotide.
53. The mixture of clause 2, wherein the mixture further comprises:
a) a fourth oligonucleotide configured to hybridize to a first sequence in a second target polynucleotide strand, wherein the first sequence in the second target polynucleotide includes a second target variant nucleotide, and wherein the fourth oligonucleotide further comprises a nucleotide residue at its 3′-end that is positioned to hybridize to the second target variant nucleotide;
b) a fifth oligonucleotide having a sequence configured to hybridize to a sequence complementary to a second sequence within the second target polynucleotide strand, wherein the second sequence of the second target polynucleotide strand is located 5′ upstream from the first sequence on the second target polynucleotide strand; and
c) a sixth oligonucleotide having a sequence configured to hybridize to a sequence complementary to a third sequence within the second target polynucleotide strand, wherein the third sequence of the second target polynucleotide strand overlaps at least partially with the first sequence on the second target polynucleotide strand and comprises the second target variant nucleotide.
54. The mixture of clause 4, wherein the mixture further comprises:
a) a fourth oligonucleotide configured to hybridize to a first sequence (F) in a second target polynucleotide strand, wherein the first sequence in the second target polynucleotide includes a second target variant nucleotide (“second variant nucleotide”), and wherein the fourth oligonucleotide further has a nucleotide residue at its 3′-end that is positioned to hybridize to the second target variant nucleotide; b) a fifth oligonucleotide configured to hybridize to a second sequence (G) in the second target polynucleotide strand, wherein the second sequence of the second target polynucleotide strand is complementary to a third sequence (H) of the second target polynucleotide strand, the third sequence of the target polynucleotide strand being present within the second target polynucleotide strand, wherein the third sequence (H) is located 5′ upstream from the first sequence (F) of the second target polynucleotide strand; and
c) a sixth oligonucleotide configured to hybridize to a fourth sequence (I) of the second target polynucleotide strand, wherein the fourth sequence (I) is complementary to a fifth sequence (J) of the second target polynucleotide strand, the fifth sequence (J) being present in the second target polynucleotide strand, wherein the fifth sequence (J) overlaps at least partially with the first sequence (F) in the second target polynucleotide strand and includes the second target variant nucleotide.
55. The mixture of clause 53 or 54, wherein the target variant nucleotide in the sixth oligonucleotide is at least 2 nucleotides from a 3′ end or a 5′ end of the sixth oligonucleotide.
56. The mixture of clause 53, 54 or 55, wherein the sixth oligonucleotide comprises a detectable label.
57. The mixture of clause 56, wherein the detectable label of the sixth oligonucleotide is a fluorescent label.
58. The mixture of clause 56 or 57, wherein the detectable label of the sixth oligonucleotide is on a first terminal nucleotide.
59. The mixture of any one of clauses 53, 54 or 56, wherein the sixth oligonucleotide further comprises a quenching moiety.
60. The mixture of clause 59, wherein the quenching moiety of the sixth oligonucleotide is on a second terminal nucleotide of the sixth oligonucleotide.
61. The mixture of clause 59 or 60, wherein the quenching moiety of the sixth oligonucleotide is capable of quenching a signal from the detectable label of the sixth oligonucleotide.
62. The mixture of clause 53 or 54 wherein the fourth and fifth oligonucleotides are extendable.
63. The mixture of clause 53 or 54 wherein the fourth and fifth oligonucleotides are primers.
64. The mixture of clause 53 or 54 wherein the sixth oligonucleotide is non-extendable.
65. The mixture of clause 53 or 54 wherein the sixth oligonucleotide is a probe. 66. The mixture of clause 53 or 54 wherein the fourth, fifth, and/or sixth oligonucleotide comprise between 10-30 nucleotides.
67. The mixture of clause 53 or 54, wherein the sixth oligonucleotide comprises a blocking moiety.
68. The mixture of clause 67, wherein the blocking moiety of the sixth oligonucleotide is a minor groove binder (MGB) moiety.
69. The mixture of clause 68, wherein the MGB moiety of the sixth oligonucleotide is located at the 3′-end and/or 3′ portion of the sixth oligonucleotide.
70. The mixture of clause 2 or 4, wherein the mixture further comprises:
a) a fourth oligonucleotide having a sequence configured to hybridize to a sequence complementary to the first oligonucleotide, wherein the fourth oligonucleotide is configured to substantially hybridize to the first sequence and comprises at its 3′ end a different nucleotide than the complement of the target variant nucleotide; and
b) a fifth oligonucleotide having a sequence configured to hybridize to a sequence having complementary to the third oligonucleotide, wherein the fifth oligonucleotide comprises a different nucleotide at the site of the target variant nucleotide.
71. The mixture of clause 70, wherein the different nucleotide is at least two nucleotides from a 3′ end thereof 72. The mixture of clause 70 or 71, wherein the fifth oligonucleotide comprises a detectable label.
73. The mixture of clause 72, wherein the detectable label of the fifth oligonucleotide is a fluorescent label.
74. The mixture of clause 72 or 73, wherein the detectable label of the fifth oligonucleotide is on a first terminal nucleotide of the fifth oligonucleotide.
74. The mixture of clause any one of clauses 72-74, wherein the detectable label on the fifth oligonucleotide is distinguishable from the detectable label on the third oligonucleotide.
75. The mixture of any one of clauses 72-74, wherein the fifth oligonucleotide further comprises a quenching moiety.
76. The mixture of clause 75, wherein the quenching moiety of the fifth oligonucleotide is on a second terminal nucleotide of the fifth oligonucleotide.
77. The mixture of clause 75 or 76, wherein the quenching moiety of the fifth oligonucleotide is capable of quenching a signal from the detectable label of the fifth oligonucleotide.
78. The mixture of clause 70 wherein the fourth oligonucleotide is extendable.
79. The mixture of clause 70 wherein the fourth oligonucleotide is a primer.
80. The mixture of clause 70 wherein the fifth oligonucleotide is non-extendable. 81. The mixture of clause 70 wherein the fifth oligonucleotide is a probe. 82. The mixture of clause 70 wherein the fourth and fifth oligonucleotide comprise between 10-40 nucleotides.
83. The mixture of clause 70, wherein the fifth oligonucleotide comprises a blocking moiety. 84. The mixture of clause 83, wherein the blocking moiety of the fifth oligonucleotide is a minor groove binder (MGB) moiety.
85. The mixture of clause 84, wherein the MGB moiety of the fifth oligonucleotide is located at the 3′-end and/or 3′ portion of the fifth oligonucleotide.
86. A mixture comprising:
a plurality of oligonucleotide sets, wherein each oligonucleotide set comprises:
a) a first oligonucleotide configured to hybridize to a first sequence in a first target polynucleotide strand, wherein the first sequence includes a target variant nucleotide, and wherein the first oligonucleotide further has a nucleotide residue at its 3′-end that is positioned to hybridize to the target variant nucleotide; and
b) a second oligonucleotide having a sequence configured to hybridize to a sequence complementary to a second sequence within the first target polynucleotide strand, wherein the second sequence of the first target polynucleotide strand is located 5′ upstream from the first sequence of the first target polynucleotide strand, wherein the first oligonucleotide of each set is configured to hybridize to a different first sequence.
87. The mixture of clause 86, wherein each oligonucleotide set further comprises:
c) a third oligonucleotide having a sequence configured to hybridize to a sequence complementary to a third sequence within the first target polynucleotide strand, wherein the third sequence of the first target polynucleotide strand overlaps at least partially with the first sequence of the first target polynucleotide strand and the third sequence includes the target variant nucleotide, wherein the third oligonucleotide of each set shares sequence similarity to a different third sequence, and wherein the third oligonucleotide of each set comprises a different and distinguishable detectable label.
88. A mixture comprising:
a plurality of oligonucleotide sets, wherein each oligonucleotide set comprises:
a) a first oligonucleotide configured to hybridize to a first sequence (A) present within a first target polynucleotide strand, wherein the first sequence includes a target variant nucleotide (“first variant nucleotide”), and wherein the first oligonucleotide further has a nucleotide residue at its 3′-end that is positioned to hybridize to the first variant nucleotide; and
b) a second oligonucleotide configured to hybridize to a second sequence (B), where the second sequence is complementary to a third sequence (C), the third sequence present within the first target polynucleotide strand, wherein the third sequence (C) is located 5′ upstream from the first sequence (A) of the first target polynucleotide strand, wherein the first oligonucleotide of each set is configured to hybridize to a different first sequence.
89. The mixture of clause 88, wherein each oligonucleotide set further comprises:
c) a third oligonucleotide configured to hybridize to a fourth sequence (D) complementary to a fifth sequence (E), the fifth sequence present in the first target polynucleotide strand, wherein the fifth sequence (E) overlaps at least partially with the first sequence (A) in the first target polynucleotide strand and includes the first target variant nucleotide, wherein the third oligonucleotide of each set shares sequence similarity to a different third sequence, and wherein the third oligonucleotide of each set comprises a different and distinguishable detectable label.
90. The mixture of any one of clauses 1-89, wherein the mixture further comprises a polymerase. 91. The mixture of clause 90, wherein the polymerase is thermostable. 92. The mixture of clause 91, wherein the mixture further comprises a hot start component.
93. The mixture of clause 92, wherein the hot start component comprises an antibody directed to the thermostable polymerase; an oligonucleotide; an aptamer; and/or a chemical modification of the polymerase.
94. The mixture of any one of clauses 1-93, wherein the mixture further comprises a source of nucleotides.
95. The mixture of any one of clauses 1-94, wherein the third oligonucleotide is a hydrolysis probe.
96. The mixture of any one of clauses 1-95, further comprising a nucleic acid sample suspected of comprising the first target polynucleotide strand.
97. The mixture of any one of clauses 1-96, wherein the target variant nucleotide is within a mutant allele. 98. The mixture of clause 97, wherein the mutant allele is either a purine-to-purine single point mutation or a pyrimidine-to-pyrimidine single point mutation at the target variant nucleotide.
99. The mixture of clause 97 or 98, wherein the mutant allele is an allele of the KRAS oncogene.
100. The mixture of clause 97, wherein the mutant allele is a stochastic mutation.
101. The mixture of any one of clauses 1-100, wherein the target variant nucleotide has an identity corresponding to a major allelic variant or a minor allelic variant.
102. The mixture of clause 101, wherein the target variant nucleotide has the identity of a minor allelic variant having a minor allelic frequency (MAF) of less than 1%.
103. The mixture of clause 102, wherein the target variant nucleotide has the identity of a minor allelic variant having a minor allelic frequency (MAF) less than 0.1%, less than 0.01%, or less than 0.001%.
104. The mixture of any one of clauses 1-103, wherein the target variant nucleotide occurs at a position of a single nucleotide polymorphism.
105. The mixture of any one of clauses 1-95, wherein the mixture is a master mix. 106. The mixture of clause 105, wherein the master mix is stable at 4 to 8° C. for up to 12 months. 107. The mixture of any one of clauses 1-104, wherein the mixture is a reaction mixture. 108. The mixture of clause 107, wherein the reaction mixture further comprises an amplicon comprising the first sequence of the first target polynucleotide strand.
109. The mixture of clause 108, wherein the reaction mixture does not include an amplicon that includes a sequence of any other, different (e.g., a second) polynucleotide strand.
110. The mixture of any one of clauses 1-109, wherein the mixture further comprises one or more of the following:
a) at least one detergent;
b) glycerol; and
c) at least one reference dye.
111. The mixture of any one of clauses 1-110, the mixture further comprising a bovine serum albumin and/or a gelatin.
112. The mixture of any one of clauses 1-106, wherein the mixture is lyophilized.
113. A kit comprising a first container containing a mixture of any preceding clause and a second container containing a control polynucleotide sample comprising a polynucleotide molecule sharing sequence similarity with the first target polynucleotide strand.
114. The kit of clause 113, wherein the control polynucleotide sample includes the entire first target polynucleotide strand.
115. The kit of clause 113, wherein the control polynucleotide sample does not share sequence similarity with the first target polynucleotide strand at the target variant nucleotide.
116. A method for detecting a target polynucleotide molecule including a target variant nucleotide in a test polynucleotide sample, the method comprising:
a) forming a reaction mixture of a test polynucleotide sample and a mixture of clause 2 or 4;
b) carrying out an amplification reaction using at least the first and second oligonucleotides as primers to produce amplicons of a target polynucleotide sequence of the target polynucleotide molecule if present in the test polynucleotide sample; and,
   c) detecting the amplicons produced in step b) by detecting a change in a detectable property of the third oligonucleotide;
wherein detecting the amplicons in step c) indicates the target polynucleotide molecule is present within the test polynucleotide sample.
   117. The method of clause 116, wherein the target polynucleotide molecule is detected in a test polynucleotide sample comprising a mixture of polynucleotide molecules, the mixture including polynucleotide molecules that include a first variant form of the target variant nucleotide (“first variant target polynucleotide molecules”) and polynucleotide molecules that include a second variant form of the target variant nucleotide (“second variant target polynucleotide molecules”).
   118. The method of clause 116 or 117, wherein the test polynucleotide sample comprises polynucleotide strands that do not include a target polynucleotide sequence (“non-target polynucleotide molecules”).
   119. The method of clause 118, wherein the test sample comprises more non-target polynucleotide molecules than target polynucleotide molecules.
   120. The method of any one of clauses 116-119, wherein the target polynucleotide molecules are mutant polynucleotide sequences.
   121. The method of any one of clauses 118-120, wherein the non-target polynucleotide molecules are major allelic or wild-type polynucleotide sequences.
   122. The method of any one of clauses 118-121, wherein the test sample comprises less than 1%, less than 0.1%, less than 0.01%, or less than 0.001% target polynucleotide molecules relative to non-target polynucleotide molecules.
   123. The method of any one of clauses 117-119, wherein the test sample comprises less than 1%, less than 0.1%, less than 0.01%, or less than 0.001% of second variant target polynucleotide molecules relative to first variant target polynucleotide molecules.
   124. The method of any one of clauses 117-119, wherein the first variant target polynucleotide molecules and/or second variant target polynucleotide molecules are mutant polynucleotide sequences.
   125. The method of any one of clauses 117-119, wherein the first variant target polynucleotide molecules and/or second variant target polynucleotide molecules are wild-type polynucleotide sequences
   126. The method of clause 117-125, further comprising enriching the number of first variant polynucleotide molecules in the polynucleotide test sample relative to the second variant polynucleotide molecules prior to steps a) through c).
   127. The method of clause 126, wherein the enriching comprises an amplification reaction comprising different conditions as compared to those used in any of steps a) through c).
   128. The method of clause 126 or 127, wherein the number of first variant polynucleotide molecules in the polynucleotide test sample are enriched by at least two-fold, four-fold, six-fold, eight-fold, or ten-fold relative to the second variant polynucleotide molecules.
   129. The method of any one of clauses 126-128, wherein the enriching is carried out using polymerase chain reaction comprising from 15-25 cycles at a temperature of 12-16 degrees higher than the calculated melting temperature (T_{M}) of the first oligonucleotide.
   130. The method of any one of clauses 126-129, wherein step b) is carried out using 35-40 cycles at a temperature near the T_{M} of the third oligonucleotide and four to six degrees below the temperature at which the enriching is carried out.
   131. The method of any one of clauses 116-130, wherein the test polynucleotide sample is derived from a mammalian or non-mammalian animal tissue or cell, or a plant tissue or cell.
   132. The method of clause 131, wherein the sample is selected from the group consisting of saliva, cheek tissue, skin, hair, blood, plasma, urine, feces, semen, and a tumor sample.
   133. The method of clause 131 or 132, wherein the polynucleotide test sample is derived from a cancer cell.
   134. The method of any one of clauses 131-133, wherein the detection of amplicons indicates the presence of cancer cells within a tissue from which the test polynucleotide sample was derived.
   135. The method of any one of clauses 116-134, wherein the target polynucleotide includes at least one mutation in Ras, EFGR, Kit, pTEN, and/or p53; and/or at least one KRAS or NRAS mutation.
   136. The method of any one of clauses 116-135, wherein the amplification reaction is a polymerase chain reaction (PCR).
   137. The method of clause 136, wherein the PCR is real-time PCR or a quantitative PCR (qPCR).
   138. The method of clause 136 or 137, wherein the third oligonucleotide has a Tₘ that is 8-12° C. above the Tₘ of the first oligonucleotide and the PCR is carried out with an annealing temperature that is within 5° C. of the Tₘ of the first oligonucleotide.
   139. The method of any one of clauses 116-138, the method being carried out using a kit that includes a first container containing at least the first oligonucleotide, the second oligonucleotide, and the third oligonucleotide and a second container containing a control polynucleotide sample including the first target polynucleotide strand.
   140. The method of any one of clauses 116-139, wherein the method detects a target variant nucleotide indicative of a mutation, and wherein the test polynucleotide sample comprises 1-10 copies of the target polynucleotide.
   141. The method of any one of clauses 116-140, wherein the target variant nucleotide includes a purine base and a corresponding wild-type nucleotide at the target variant nucleotide position includes a different purine base.
   142. The method of any one of clauses 116-140, wherein the target variant nucleotide includes a pyrimidine base and a corresponding wild-type nucleotide at the target variant nucleotide position includes a different pyrimidine base.
   143. A method for detecting a target polynucleotide molecule including a target variant nucleotide in a test polynucleotide sample, the method comprising:
a) forming a reaction mixture of a test polynucleotide sample and a mixture of any one of clause 1-94, 104, 105, and 109-111;
b) carrying out an amplification reaction using at least the first and second oligonucleotides as primers to produce amplicons of a target polynucleotide sequence of the target polynucleotide molecule if present in the test polynucleotide sample; and,
   c) detecting the amplicons produced in step b) by detecting a change in a detectable property of the third oligonucleotide;
wherein detecting the amplicons in step c) indicates the target polynucleotide molecule is present within the test polynucleotide sample.
   144. A method for detecting at least two different target polynucleotide molecules, each including a target variant nucleotide, in a test polynucleotide sample, the method comprising:
a) forming a reaction mixture of a test polynucleotide sample and a mixture of clause 53 or 54;
b) carrying out an amplification reaction using the first and second oligonucleotides as primers to produce an amplicon of a first target polynucleotide sequence of a first target polynucleotide molecule if present in the test polynucleotide sample;
c) carrying out an amplification reaction using the fourth and fifth oligonucleotides as primers to produce a second amplicon of a second target polynucleotide sequence of a second target polynucleotide molecule if present in the test polynucleotide sample,
   d) detecting the amplicons produced in steps b) and c) by detecting a change in a detectable property of the third and sixth oligonucleotides;
wherein detecting the amplicons in step d) indicates the first and/or second target polynucleotide molecule is present within the test polynucleotide sample.
   145. The method of clause 144, wherein the detectable property of the third and sixth oligonucleotides is a fluorescent label.
   146. The method of clause 145, wherein the fluorescent label on each of the third and sixth oligonucleotides is different.
   147. The mixture of any one of clauses 1-112, wherein the mixture is used to detect one or more mutations listed in Table 1 and/or Table 2.
   148. The kit of any one of clauses 113-115, wherein the kit is used for detection of one or more mutations listed in Table 1 and/or Table 2.
   149. The method of any one of clauses 116-146, wherein the method is used to detect one or more mutations listed in Table 1 and/or Table 2.

What is claimed is:

1. A mixture comprising:
a) a first oligonucleotide configured to hybridize to a first sequence in a first target polynucleotide strand, wherein the first sequence includes a target variant nucleotide, and wherein the first oligonucleotide further has a nucleotide residue at its 3′-end that is positioned to hybridize to the target variant nucleotide; and
b) a second oligonucleotide having a sequence configured to hybridize to a sequence complementary to a second sequence within the first target polynucleotide strand, wherein the second sequence of the first target polynucleotide strand is located 5′ upstream from the first sequence of the first target polynucleotide strand.

2. The mixture of claim 1, further comprising:
c) a third oligonucleotide having a sequence configured to hybridize to a sequence complementary to a third sequence within the first target polynucleotide strand, wherein the third sequence of the first target polynucleotide strand overlaps at least partially with the first sequence of the first target polynucleotide strand and the third sequence includes the target variant nucleotide.

3. A mixture comprising:
a) a first oligonucleotide configured to hybridize to a first sequence (A) present within a first target polynucleotide strand, wherein the first sequence includes a target variant nucleotide (“first variant nucleotide”), and wherein the first oligonucleotide further has a nucleotide residue at its 3′-end that is positioned to hybridize to the first variant nucleotide; and
b) a second oligonucleotide configured to hybridize to a second sequence (B), wherein the second sequence is complementary to a third sequence (C), the third sequence being present within the first target polynucleotide strand, wherein the third sequence (C) is located 5′ upstream from the first sequence (A) of the first target polynucleotide strand.

4. The mixture of claim 2, further comprising:
c) a third oligonucleotide configured to hybridize to a fourth sequence (D) complementary to a fifth sequence (E), the fifth sequence present in the first target polynucleotide strand, wherein the fifth sequence (E) overlaps at least partially with the first sequence (A) in the first target polynucleotide strand and includes the first target variant nucleotide.

5. The mixture of claim 2 or 4, wherein the target variant nucleotide in the third oligonucleotide is at least two nucleotides from a 3′ end or a 5′ end of the third oligonucleotide.

6. The mixture of any one of claims 2, 4 and 5, wherein the third oligonucleotide comprises a detectable label.

7. The mixture of claim 6, wherein the detectable label is a fluorescent label.

8. The mixture of claim 6 or 7, wherein the detectable label is on a first terminal nucleotide.

9. The mixture of any one of claims 6-8, wherein the third oligonucleotide further comprises a quenching moiety.

10. The mixture of claim 9, wherein the quenching moiety is on a second terminal nucleotide of the third oligonucleotide.

11. The mixture of claim 9 or 10, wherein the quenching moiety is capable of quenching a signal from the detectable label.

12. The mixture of claim 10, wherein the first terminal nucleotide is the 5′ terminal nucleotide.

13. The mixture of claim 10, wherein the second terminal nucleotide is the 3′ terminal nucleotide.

14. The mixture of claim 2 or 4, wherein the mixture comprises a single-stranded polynucleotide molecule including the first target polynucleotide strand.

15. The mixture of claim 4, wherein sequences A, E and C are located within a single-stranded polynucleotide molecule on the first target polynucleotide strand.

16. The mixture of claim 2 or 4, wherein the mixture comprises a double-stranded polynucleotide molecule including the first target polynucleotide strand and a first target complement polynucleotide strand, wherein the first target complement polynucleotide strand is substantially complementary to the first target polynucleotide strand.

17. The mixture of claim 4, wherein the mixture comprises a double-stranded polynucleotide molecule including the first target polynucleotide strand and a first target complement polynucleotide strand, wherein the first target complement polynucleotide strand is substantially complementary to the first target polynucleotide strand, and wherein sequences D and B are located within the double-stranded polynucleotide molecule on the target complement polynucleotide strand and sequences A, E, and C are located within the double-stranded polynucleotide molecule on the target polynucleotide strand.

18. The mixture of claim 2 or 4, wherein the mixture comprises a double-stranded polynucleotide molecule including the first target polynucleotide strand and a first target complement polynucleotide strand, wherein the first target complement polynucleotide strand is substantially complementary to the first target polynucleotide strand, and a double-stranded polynucleotide molecule including a variant polynucleotide strand and a variant complement polynucleotide strand, wherein the variant polynucleotide strand is substantially identical to the target polynucleotide strand and comprises a different nucleotide at the target variant nucleotide than the target polynucleotide strand, and wherein the variant complement polynucleotide strand is substantially complementary to the variant polynucleotide strand.

19. The mixture of claim 2 or 4, wherein the third oligonucleotide comprises 3 to 6 contiguous nucleotides of the first sequence.

20. The mixture of claim 2 or 4, wherein the third oligonucleotide further comprises a sequence of nucleotides of the first target polynucleotide strand that does not overlap with a sequence of nucleotides of the first sequence.

21. The mixture of claim 2 or 4, wherein the mixture does not comprise a fourth oligonucleotide.

22. The mixture of claim 2 or 4, wherein the mixture does not comprise a fourth oligonucleotide comprising a detectable label.

23. The mixture of claim 2 or 4, wherein the mixture does not comprise a fourth oligonucleotide that binds to the first target polynucleotide strand.

24. The mixture of claim 2 or 4, wherein the mixture does not comprise a fourth oligonucleotide having a detectable label and a sequence that binds to the first target polynucleotide strand.

25. The mixture of claim 2 or 4, wherein the third oligonucleotide is the only oligonucleotide in the mixture having a detectable label and a sequence configured to hybridize to a sequence of the target polynucleotide strand.

26. The mixture of claim 2 or 4, wherein the mixture further comprises between 30 mM and 80 mM potassium chloride.

27. The mixture of claim 26, wherein the concentration of potassium chloride is at least 40 mM.

28. The mixture of claim 26, wherein the concentration of potassium chloride is less than 70 mM.

29. The mixture of claim 26, wherein the concentration of potassium chloride is at least 40 mM and less than 70 mM.

30. The mixture of claim 2 or 4, wherein the mixture further comprises between 10 mM and 40 mM ammonium sulfate.

31. The mixture of claim 30, wherein the concentration of ammonium sulfate is at least 20 mM.

32. The mixture of claim 30, wherein the concentration of ammonium sulfate is less than 35 mM.

33. The mixture of claim 30, wherein the concentration of ammonium sulfate is at least 20 mM and less than 35 mM.

34. The mixture of claim 30, wherein the concentration of ammonium sulfate is 20 to 25 mM.

35. The mixture of claim 2 or 4, wherein the mixture further comprises:
a) a concentration of potassium chloride between 30 mM and 80 mM; and,
b) a concentration of ammonium sulfate between 10 mM and 40 mM.

36. The mixture of claim 35, wherein the concentration of potassium chloride is 40 mM to 70 mM and the concentration of ammonium sulfate is 20 mM to 35 mM.

37. The mixture of claim 35, wherein the concentration of potassium chloride is 40 mM to 48 mM and the concentration of ammonium sulfate is 20 to 24 mM.

38. The mixture of claim 35, wherein the concentration of potassium chloride is 45 mM and the concentration of ammonium sulfate is 22 mM.

39. The mixture of claim 6, wherein the detectable label is selected from the group consisting of a DNA-binding dye, reporter dye, fluorescent probe, 6-carboxyfluorescein (FAM™), tetrachlorofluorescin (TET™), 6-Carboxy-4′,5′-Dichloro-2′,7′-Dimethoxyfluorescein, Succinimidyl Ester (JOE™), VIC™, a sulfonate derivative of a fluorescein dye with SO₃ instead of the carboxylate group, a phosphoramidite form of fluorescein, a phosphoramidite form of CY5, a non-FRET label, a ferrocene reagent, ABY™ NED™ JUN™ Fluor®488, AlexaFluor®532, AlexaFluor®546, AlexaFluor®594, AlexaFluor®647, AlexaFluor® 660, TYE™ 563, TYE™665, TYE™705, and combinations thereof.

40. The mixture of any one of claims 9-11, wherein the quenching moiety is selected from the group consisting of tetramethylrhodamine (TAMRA), a non-fluorescent quencher (NFQ), Black Hole Quencher, Iowa Black Quencher, QSY quencher, QSY7 quencher, QSY21 quencher, Dabsyl and/or Dabsyl sulfonate/carboxylate quenchers.

41. The mixture of claim 2 or 4, wherein the Tₘ of the third oligonucleotide is at least 5° C. and no more than 25° C. higher than the Tₘ of the first oligonucleotide.

42. The mixture of claim 41, wherein the Tₘ of the third oligonucleotide is at least 8° C. and no more than 12° C. higher than the Tₘ of the first oligonucleotide.

43. The mixture of claim 2 or 4, wherein the Tₘ of the first oligonucleotide is within 5° C. of the Tₘ of the second oligonucleotide.

44. The mixture of any one of claim 42 or 43, wherein the Tₘ of the first oligonucleotide is 45 to 60° C. and the Tₘ of the second oligonucleotide is 45 to 60° C.

45. The mixture of any one of claims 1-4, wherein the first and second oligonucleotides are extendable.

46. The mixture of any one of claims 1-4, wherein the first and second oligonucleotides are primers.

47. The mixture of claim 2 or 4, wherein the third oligonucleotide is non-extendable.

48. The mixture of claim 2 or 4, wherein the third oligonucleotide is a probe.

49. The mixture of claim 2 or 4, wherein the first, second, and/or third oligonucleotides comprise between 10-40 nucleotides.

50. The mixture of claim 2 or 4, wherein the third oligonucleotide comprises a blocking moiety.

51. The mixture of claim 50, wherein the blocking moiety is a minor groove binder (MGB) moiety.

52. The mixture of claim 51, wherein the MGB moiety is located at the 3′-end and/or 3′ portion of the third oligonucleotide.

53. The mixture of claim 2, wherein the mixture further comprises:
a) a fourth oligonucleotide configured to hybridize to a first sequence in a second target polynucleotide strand, wherein the first sequence in the second target polynucleotide includes a second target variant nucleotide, and wherein the fourth oligonucleotide further comprises a nucleotide residue at its 3′-end that is positioned to hybridize to the second target variant nucleotide;
b) a fifth oligonucleotide having a sequence configured to hybridize to a sequence complementary to a second sequence within the second target polynucleotide strand, wherein the second sequence of the second target polynucleotide strand is located 5′ upstream from the first sequence on the second target polynucleotide strand; and
c) a sixth oligonucleotide having a sequence configured to hybridize to a sequence complementary to a third sequence within the second target polynucleotide strand, wherein the third sequence of the second target polynucleotide strand overlaps at least partially with the first sequence on the second target polynucleotide strand and comprises the second target variant nucleotide.

54. The mixture of claim 4, wherein the mixture further comprises:
a) a fourth oligonucleotide configured to hybridize to a first sequence (F) in a second target polynucleotide strand, wherein the first sequence in the second target polynucleotide includes a second target variant nucleotide (“second variant nucleotide”), and wherein the fourth oligonucleotide further has a nucleotide residue at its 3′-end that is positioned to hybridize to the second target variant nucleotide;
b) a fifth oligonucleotide configured to hybridize to a second sequence (G) in the second target polynucleotide strand, wherein the second sequence of the second target polynucleotide strand is complementary to a third sequence (H) of the second target polynucleotide strand, the third sequence of the target polynucleotide strand being present within the second target polynucleotide strand, wherein the third sequence (H) is located 5′ upstream from the first sequence (F) of the second target polynucleotide strand; and
c) a sixth oligonucleotide configured to hybridize to a fourth sequence (I) of the second target polynucleotide strand, wherein the fourth sequence (I) is complementary to a fifth sequence (J) of the second target polynucleotide strand, the fifth sequence (J) being present in the second target polynucleotide strand, wherein the fifth sequence (J) overlaps at least partially with the first sequence (F) in the second target polynucleotide strand and includes the second target variant nucleotide.

55. The mixture of claim 53 or 54, wherein the target variant nucleotide in the sixth oligonucleotide is at least 2 nucleotides from a 3′ end or a 5′ end of the sixth oligonucleotide.

56. The mixture of claim 53, 54 or 55, wherein the sixth oligonucleotide comprises a detectable label.

57. The mixture of claim 56, wherein the detectable label of the sixth oligonucleotide is a fluorescent label.

58. The mixture of claim 56 or 57, wherein the detectable label of the sixth oligonucleotide is on a first terminal nucleotide.

59. The mixture of any one of claim 53, 54 or 56, wherein the sixth oligonucleotide further comprises a quenching moiety.

60. The mixture of claim 59, wherein the quenching moiety of the sixth oligonucleotide is on a second terminal nucleotide of the sixth oligonucleotide.

61. The mixture of claim 59 or 60, wherein the quenching moiety of the sixth oligonucleotide is capable of quenching a signal from the detectable label of the sixth oligonucleotide.

62. The mixture of claim 53 or 54 wherein the fourth and fifth oligonucleotides are extendable.

63. The mixture of claim 53 or 54 wherein the fourth and fifth oligonucleotides are primers.

64. The mixture of claim 53 or 54 wherein the sixth oligonucleotide is non-extendable.

65. The mixture of claim 53 or 54 wherein the sixth oligonucleotide is a probe.

66. The mixture of claim 53 or 54 wherein the fourth, fifth, and/or sixth oligonucleotide comprise between 10-30 nucleotides.

67. The mixture of claim 53 or 54, wherein the sixth oligonucleotide comprises a blocking moiety.

68. The mixture of claim 67, wherein the blocking moiety of the sixth oligonucleotide is a minor groove binder (MGB) moiety.

69. The mixture of claim 68, wherein the MGB moiety of the sixth oligonucleotide is located at the 3′-end and/or 3′ portion of the sixth oligonucleotide.

70. The mixture of claim 2 or 4, wherein the mixture further comprises:
a) a fourth oligonucleotide having a sequence configured to hybridize to a sequence complementary to the first oligonucleotide, wherein the fourth oligonucleotide is configured to substantially hybridize to the first sequence and comprises at its 3′ end a different nucleotide than the complement of the target variant nucleotide; and
b) a fifth oligonucleotide having a sequence configured to hybridize to a sequence having complementary to the third oligonucleotide, wherein the fifth oligonucleotide comprises a different nucleotide at the site of the target variant nucleotide.

71. The mixture of claim 70, wherein the different nucleotide is at least two nucleotides from a 3′ end thereof.

72. The mixture of claim 70 or 71, wherein the fifth oligonucleotide comprises a detectable label.

73. The mixture of claim 72, wherein the detectable label of the fifth oligonucleotide is a fluorescent label.

74. The mixture of claim 72 or 73, wherein the detectable label of the fifth oligonucleotide is on a first terminal nucleotide of the fifth oligonucleotide.

74. The mixture of claim any one of claims 72-74, wherein the detectable label on the fifth oligonucleotide is distinguishable from the detectable label on the third oligonucleotide.

75. The mixture of any one of claims 72-74, wherein the fifth oligonucleotide further comprises a quenching moiety.

76. The mixture of claim 75, wherein the quenching moiety of the fifth oligonucleotide is on a second terminal nucleotide of the fifth oligonucleotide.

77. The mixture of claim 75 or 76, wherein the quenching moiety of the fifth oligonucleotide is capable of quenching a signal from the detectable label of the fifth oligonucleotide.

78. The mixture of claim 70 wherein the fourth oligonucleotide is extendable.

79. The mixture of claim 70 wherein the fourth oligonucleotide is a primer.

80. The mixture of claim 70 wherein the fifth oligonucleotide is non-extendable.

81. The mixture of claim 70 wherein the fifth oligonucleotide is a probe.

82. The mixture of claim 70 wherein the fourth and fifth oligonucleotide comprise between 10-40 nucleotides.

83. The mixture of claim 70, wherein the fifth oligonucleotide comprises a blocking moiety.

84. The mixture of claim 83, wherein the blocking moiety of the fifth oligonucleotide is a minor groove binder (MGB) moiety.

85. The mixture of claim 84, wherein the MGB moiety of the fifth oligonucleotide is located at the 3′-end and/or 3′ portion of the fifth oligonucleotide.

86. A mixture comprising:
a plurality of oligonucleotide sets, wherein each oligonucleotide set comprises:
a) a first oligonucleotide configured to hybridize to a first sequence in a first target polynucleotide strand, wherein the first sequence includes a target variant nucleotide, and wherein the first oligonucleotide further has a nucleotide residue at its 3′-end that is positioned to hybridize to the target variant nucleotide; and
b) a second oligonucleotide having a sequence configured to hybridize to a sequence complementary to a second sequence within the first target polynucleotide strand, wherein the second sequence of the first target polynucleotide strand is located 5′ upstream from the first sequence of the first target polynucleotide strand,
wherein the first oligonucleotide of each set is configured to hybridize to a different first sequence.

87. The mixture of claim 86, wherein each oligonucleotide set further comprises:
c) a third oligonucleotide having a sequence configured to hybridize to a sequence complementary to a third sequence within the first target polynucleotide strand, wherein the third sequence of the first target polynucleotide strand overlaps at least partially with the first sequence of the first target polynucleotide strand and the third sequence includes the target variant nucleotide,
wherein the third oligonucleotide of each set shares sequence similarity to a different third sequence, and
wherein the third oligonucleotide of each set comprises a different and distinguishable detectable label.

88. A mixture comprising:
a plurality of oligonucleotide sets, wherein each oligonucleotide set comprises:
a) a first oligonucleotide configured to hybridize to a first sequence (A) present within a first target polynucleotide strand, wherein the first sequence includes a target variant nucleotide (“first variant nucleotide”), and wherein the first oligonucleotide further has a nucleotide residue at its 3′-end that is positioned to hybridize to the first variant nucleotide; and
b) a second oligonucleotide configured to hybridize to a second sequence (B), where the second sequence is complementary to a third sequence (C), the third sequence present within the first target polynucleotide strand, wherein the third sequence (C) is located 5′ upstream from the first sequence (A) of the first target polynucleotide strand,
wherein the first oligonucleotide of each set is configured to hybridize to a different first sequence.

89. The mixture of claim 88, wherein each oligonucleotide set further comprises:
c) a third oligonucleotide configured to hybridize to a fourth sequence (D) complementary to a fifth sequence (E), the fifth sequence present in the first target polynucleotide strand, wherein the fifth sequence (E) overlaps at least partially with the first sequence (A) in the first target polynucleotide strand and includes the first target variant nucleotide,
wherein the third oligonucleotide of each set shares sequence similarity to a different third sequence, and
wherein the third oligonucleotide of each set comprises a different and distinguishable detectable label.

90. The mixture of any one of claims 1-89, wherein the mixture further comprises a polymerase.

91. The mixture of claim 90, wherein the polymerase is thermostable.

92. The mixture of claim 91, wherein the mixture further comprises a hot start component.

93. The mixture of claim 92, wherein the hot start component comprises an antibody directed to the thermostable polymerase; an oligonucleotide; an aptamer; and/or a chemical modification of the polymerase.

94. The mixture of any one of claims 1-93, wherein the mixture further comprises a source of nucleotides.

95. The mixture of any one of claims 1-94, wherein the third oligonucleotide is a hydrolysis probe.

96. The mixture of any one of claims 1-95, further comprising a nucleic acid sample suspected of comprising the first target polynucleotide strand.

97. The mixture of any one of claims 1-96, wherein the target variant nucleotide is within a mutant allele.

98. The mixture of claim 97, wherein the mutant allele is either a purine-to-purine single point mutation or a pyrimidine-to-pyrimidine single point mutation at the target variant nucleotide.

99. The mixture of claim 97 or 98, wherein the mutant allele is an allele of the KRAS oncogene.

100. The mixture of claim 97, wherein the mutant allele is a stochastic mutation.

101. The mixture of any one of claims 1-100, wherein the target variant nucleotide has an identity corresponding to a major allelic variant or a minor allelic variant.

102. The mixture of claim 101, wherein the target variant nucleotide has the identity of a minor allelic variant having a minor allelic frequency (MAF) of less than 1%.

103. The mixture of claim 102, wherein the target variant nucleotide has the identity of a minor allelic variant having a minor allelic frequency (MAF) less than 0.1%, less than 0.01%, or less than 0.001%.

104. The mixture of any one of claims 1-103, wherein the target variant nucleotide occurs at a position of a single nucleotide polymorphism.

105. The mixture of any one of claims 1-95, wherein the mixture is a master mix.

106. The mixture of claim 105, wherein the master mix is stable at 4 to 8° C. for up to 12 months.

107. The mixture of any one of claims 1-104, wherein the mixture is a reaction mixture.

108. The mixture of claim 107, wherein the reaction mixture further comprises an amplicon comprising the first sequence of the first target polynucleotide strand.

109. The mixture of claim 108, wherein the reaction mixture does not include an amplicon that includes a sequence of any other, different (e.g., a second) polynucleotide strand.

110. The mixture of any one of claims 1-109, wherein the mixture further comprises one or more of the following:
a) at least one detergent;
b) glycerol; and
c) at least one reference dye.

111. The mixture of any one of claims 1-110, the mixture further comprising a bovine serum albumin and/or a gelatin.

112. The mixture of any one of claims 1-106, wherein the mixture is lyophilized.

113. A kit comprising a first container containing a mixture of any preceding claim and a second container containing a control polynucleotide sample comprising a polynucleotide molecule sharing sequence similarity with the first target polynucleotide strand.

114. The kit of claim 113, wherein the control polynucleotide sample includes the entire first target polynucleotide strand.

115. The kit of claim 113, wherein the control polynucleotide sample does not share sequence similarity with the first target polynucleotide strand at the target variant nucleotide.

116. A method for detecting a target polynucleotide molecule including a target variant nucleotide in a test polynucleotide sample, the method comprising:
a) forming a reaction mixture of a test polynucleotide sample and a mixture of claim 2 or 4;
b) carrying out an amplification reaction using at least the first and second oligonucleotides as primers to produce amplicons of a target polynucleotide sequence of the target polynucleotide molecule if present in the test polynucleotide sample; and,
c) detecting the amplicons produced in step b) by detecting a change in a detectable property of the third oligonucleotide;
wherein detecting the amplicons in step c) indicates the target polynucleotide molecule is present within the test polynucleotide sample.

117. The method of claim 116, wherein the target polynucleotide molecule is detected in a test polynucleotide sample comprising a mixture of polynucleotide molecules, the mixture including polynucleotide molecules that include a first variant form of the target variant nucleotide (“first variant target polynucleotide molecules”) and polynucleotide molecules that include a second variant form of the target variant nucleotide (“second variant target polynucleotide molecules”).

118. The method of claim 116 or 117, wherein the test polynucleotide sample comprises polynucleotide strands that do not include a target polynucleotide sequence (“non-target polynucleotide molecules”).

119. The method of claim 118, wherein the test sample comprises more non-target polynucleotide molecules than target polynucleotide molecules.

120. The method of any one of claims 116-119, wherein the target polynucleotide molecules are mutant polynucleotide sequences.

121. The method of any one of claims 118-120, wherein the non-target polynucleotide molecules are major allelic or wild-type polynucleotide sequences.

122. The method of any one of claims 118-121, wherein the test sample comprises less than 1%, less than 0.1%, less than 0.01%, or less than 0.001% target polynucleotide molecules relative to non-target polynucleotide molecules.

123. The method of any one of claims 117-119, wherein the test sample comprises less than 1%, less than 0.1%, less than 0.01%, or less than 0.001% of second variant target polynucleotide molecules relative to first variant target polynucleotide molecules.

124. The method of any one of claims 117-119, wherein the first variant target polynucleotide molecules and/or second variant target polynucleotide molecules are mutant polynucleotide sequences.

125. The method of any one of claims 117-119, wherein the first variant target polynucleotide molecules and/or second variant target polynucleotide molecules are wild-type polynucleotide sequences

126. The method of claims 117-125, further comprising enriching the number of first variant polynucleotide molecules in the polynucleotide test sample relative to the second variant polynucleotide molecules prior to steps a) through c).

127. The method of claim 126, wherein the enriching comprises an amplification reaction comprising different conditions as compared to those used in any of steps a) through c).

128. The method of claim 126 or 127, wherein the number of first variant polynucleotide molecules in the polynucleotide test sample are enriched by at least two-fold, four-fold, six-fold, eight-fold, or ten-fold relative to the second variant polynucleotide molecules.

129. The method of any one of claims 126-128, wherein the enriching is carried out using polymerase chain reaction comprising from 15-25 cycles at a temperature of 12-16 degrees higher than the calculated melting temperature (T_{M}) of the first oligonucleotide.

130. The method of any one of claims 126-129, wherein step b) is carried out using 35-40 cycles at a temperature near the T_{M} of the third oligonucleotide and four to six degrees below the temperature at which the enriching is carried out.

131. The method of any one of claims 116-130, wherein the test polynucleotide sample is derived from a mammalian or non-mammalian animal tissue or cell, or a plant tissue or cell.

132. The method of claim 131, wherein the sample is selected from the group consisting of saliva, cheek tissue, skin, hair, blood, plasma, urine, feces, semen, and a tumor sample.

133. The method of claim 131 or 132, wherein the polynucleotide test sample is derived from a cancer cell.

134. The method of any one of claims 131-133, wherein the detection of amplicons indicates the presence of cancer cells within a tissue from which the test polynucleotide sample was derived.

135. The method of any one of claims 116-134, wherein the target polynucleotide includes at least one mutation in Ras, EFGR, Kit, pTEN, and/or p53; and/or at least one KRAS or NRAS mutation.

136. The method of any one of claims 116-135, wherein the amplification reaction is a polymerase chain reaction (PCR).

137. The method of claim 136, wherein the PCR is real-time PCR or a quantitative PCR (qPCR).

138. The method of claim 136 or 137, wherein the third oligonucleotide has a Tₘ that is 8-12° C. above the Tₘ of the first oligonucleotide and the PCR is carried out with an annealing temperature that is within 5° C. of the Tₘ of the first oligonucleotide.

139. The method of any one of claims 116-138, the method being carried out using a kit that includes a first container containing at least the first oligonucleotide, the second oligonucleotide, and the third oligonucleotide and a second container containing a control polynucleotide sample including the first target polynucleotide strand.

140. The method of any one of claims 116-139, wherein the method detects a target variant nucleotide indicative of a mutation, and wherein the test polynucleotide sample comprises 1-10 copies of the target polynucleotide.

141. The method of any one of claims 116-140, wherein the target variant nucleotide includes a purine base and a corresponding wild-type nucleotide at the target variant nucleotide position includes a different purine base.

142. The method of any one of claims 116-140, wherein the target variant nucleotide includes a pyrimidine base and a corresponding wild-type nucleotide at the target variant nucleotide position includes a different pyrimidine base.

143. A method for detecting a target polynucleotide molecule including a target variant nucleotide in a test polynucleotide sample, the method comprising:
a) forming a reaction mixture of a test polynucleotide sample and a mixture of any one of claims 1-94, 104, 105, and 109-111;
b) carrying out an amplification reaction using at least the first and second oligonucleotides as primers to produce amplicons of a target polynucleotide sequence of the target polynucleotide molecule if present in the test polynucleotide sample; and,
c) detecting the amplicons produced in step b) by detecting a change in a detectable property of the third oligonucleotide;
wherein detecting the amplicons in step c) indicates the target polynucleotide molecule is present within the test polynucleotide sample.

144. A method for detecting at least two different target polynucleotide molecules, each including a target variant nucleotide, in a test polynucleotide sample, the method comprising:
a) forming a reaction mixture of a test polynucleotide sample and a mixture of claim 53 or 54;
b) carrying out an amplification reaction using the first and second oligonucleotides as primers to produce an amplicon of a first target polynucleotide sequence of a first target polynucleotide molecule if present in the test polynucleotide sample;
c) carrying out an amplification reaction using the fourth and fifth oligonucleotides as primers to produce a second amplicon of a second target polynucleotide sequence of a second target polynucleotide molecule if present in the test polynucleotide sample,
d) detecting the amplicons produced in steps b) and c) by detecting a change in a detectable property of the third and sixth oligonucleotides;
wherein detecting the amplicons in step d) indicates the first and/or second target polynucleotide molecule is present within the test polynucleotide sample.

145. The method of claim 144, wherein the detectable property of the third and sixth oligonucleotides is a fluorescent label.

146. The method of claim 145, wherein the fluorescent label on each of the third and sixth oligonucleotides is different.

147. The mixture of any one of claims 1-112, wherein the mixture is used to detect one or more mutations listed in Table 1 and/or Table 2.

148. The kit of any one of claims 113-115, wherein the kit is used for detection of one or more mutations listed in Table 1 and/or Table 2.

149. The method of any one of claims 116-146, wherein the method is used to detect one or more mutations listed in Table 1 and/or Table 2.
